(19) United States
(12) Patent Application Publication
(10) Pub. No.: US 2024/0002383 A1
(43) Pub. Date: Jan. 4, 2024

(54) COMPOUNDS AND THEIR USE AS VACCINE ADJUVANTS

(71) Applicants: Fulgent Genetics, Inc., Temple City, CA (US); Fudan Univeristy, Shanghai (CN)

(72) Inventors: Lu LU, Shanghai (CN); Shibo JIANG, Shanghai (CN); Zezhong LIU, Shanghai (CN); Ming HSEIH, Temple City, CA (US); Yilong ZHANG, Temple City, CA (US); Jie ZHOU, Temple City, CA (US); Qian WANG, Temple City, CA (US); Xinling WANG, Temple City, CA (US); Wei XU, Temple City, CA (US)

(21) Appl. No.: 18/465,513

(22) Filed: Sep. 12, 2023

Related U.S. Application Data
(63) Continuation of application No. PCT/CN2022/094090, filed on May 20, 2022.

(30) Foreign Application Priority Data
May 24, 2021 (WO) PCT/CN2021/095498

(51) Int. Cl. C07D 471/04; A61K 39/145; A61P 31/16; A61P 31/22; A61K 39/39; A61K 39/215; A61K 39/285

(52) U.S. Cl. CPC C07D 471/04; A61K 39/145; A61P 31/16; A61P 31/22; A61K 39/39; A61K 39/215; A61K 39/285; A61K 2039/5254

(57) ABSTRACT

Provided herein are a series of compounds and their use as an adjuvant. Provided herein are the compounds, a composition comprising the compounds, and the use thereof. These compounds can be used as an adjuvant for a vaccine, and compared to the conventional aluminum adjuvant, the compounds can significantly improve the cellular and humoral immune responses to a vaccine. The compounds as an adjuvant can increase a broad-spectrum protection against various corona viruses such as SARS virus, influenza viruses, and HIV viruses, and significantly enhance persistence of immunoprotection of vaccines.

### PRIORITY

This application is a continuation of PCT/CN2022/094090, filed May 20, 2022 which claims the priority of the PCT application No. PCT/CN2021/095498, filed on May 24, 2021 and titled “Compounds and their use as vaccine adjuvants”, both of which are incorporated herein by reference in their entirety.

### FIELD OF INVENTION

The disclosure relates to the field of biomedicine, in particular to a series of small molecule STING agonists.

### BACKGROUND OF INVENTION

Vaccines are considered the most powerful weapon to prevent the spread of infectious diseases. After the outbreak of the Corona Virus Disease 2019 (COVID-19), a variety of vaccines against the coronavirus SARS-CoV-2 have quickly entered clinical trials, including mRNA vaccines, DNA vaccines, inactivated vaccines, viral vector vaccines, etc. The main components of these vaccines are the spike protein or receptor-binding domain (RBD) of the coronavirus. At present, many of COVID-19 vaccines have proven protective effects on SARS-CoV-2 infection in human ACE2 transgenic mice and non-human primate models. Although more than a dozen vaccines now have been authorized around the globe, there are still some urgent problems that need resolving. For example, as the virus spreads in the population, the coronavirus continues to mutate. The newly mutated virus strains have brought severe challenges to the currently marketed vaccines. Studies now have shown that the SARS-CoV-2 mutant strain in the UK and the SARS-CoV-2 mutant strain in South Africa have produced some immune escape phenomena against some existing vaccines on the market (Wang et al. Mrna vaccine-elicited antibodies to SARS-CoV-2 and circulating variants, medRxiv, 2021). In addition, in the past 20 years, three highly pathogenic coronaviruses including SARS-CoV, SARS-CoV-2 and MERS-CoV have appeared and broke out one after another. At present, some coronaviruses similar to SARS from bats such as Rs3367 and WIV1 strains have been found, which suggests that SARS-related coronaviruses may still appear suddenly and spread like SARS-CoV-2 in population in the future. At present, only a few studies have shown that vaccines based on SARS-CoV-2 antigens can produce weak cross-neutralizing antibody protection against SARS-CoV and SARS-related coronavirus infections (Liu Z et. al. RBD-Fc-based COVID-19 vaccine candidate induces highly potent SARS-CoV-2 neutralizing antibody response, Signal Transduct Target Ther, 2020). It is also controversial whether the sera of patients who have recovered from the SARS-CoV infection can neutralize SARS-CoV-2. Studies have shown that the sera from patients infected with SARS-CoV can cross-neutralize SARS-CoV-2, but the neutralization activity is weak (Zhu, Y et al. Cross-reactive neutralization of SARS-CoV-2 by serum antibodies from recovered SARS patients and immunized animals. Sci Adv, 2020). Another study shows that the sera from patients who have recovered from the SARS-CoV infection cannot effectively neutralize the SARS-CoV-2 virus (Wang, Y. et al. Kinetics of viral load and antibody response in relation to COVID-19 severity. J Clin Invest, 2020). These results suggest that it is difficult to produce a broad-spectrum, long-lasting, and strong protective immune response to SARS-related coronaviruses in body through common vaccination strategies or natural infection pathways. Therefore, the development of a broad-spectrum, long-lasting and potent universal vaccine against SARS-related viruses is essential to combat the epidemic of the current SARS-CoV-2, including mutants of SARS-CoV-2, and SARS-related viruses that may appear in the future.

Adjuvants are essential to enhance the immunoprotection of protein subunit vaccines or inactivated vaccines. At present, the most commonly used adjuvant is the aluminum adjuvant. Recently, a variety of protein subunit vaccines in clinical trials use the aluminum adjuvant. Although the aluminum adjuvant is safe, the aluminum adjuvant mainly enhances the antibody humoral immune response, and the cellular immune response also plays a vital role in resisting viral infection processes. Therefore, developing new adjuvants, especially small-molecule immune enhancers, to enhance the immunoprotective effect of subunit vaccines or inactivated vaccines against SARS-CoV-2, induce more types of immune responses and extend the persistence of immunity is currently a frontier hotspot.

### SUMMARY OF INVENTION

In recent years, STING agonists have been found to have the potential to be used as vaccine adjuvants. The present inventors previously used nano-encapsulated STING agonist cGAMP lung bionic particles as an adjuvant for influenza vaccines. Vaccination with intranasal drops can produce a strong and broad-spectrum immunoprotection against influenza viruses in mice (Wang, J. et al. Pulmonary surfactant-biomimetic nanoparticles potentiate heterosubtypic influenza immunity. Science, 2020). However, cGAMP is less effective by intramuscular injection.

In the present disclosure, a new STING small molecule agonist was used as an adjuvant for the protein subunit RBD-Fc vaccine against SARS-CoV-2. In animal models using mice, rabbits, and rhesus monkeys, the STING agonist-adjuvanted RBD-Fc strongly activated the cellular and humoral immune responses, and produced a broad-spectrum, potent, and long-lasting immunoprotection against SARS-CoV-2, its mutants, SARS-CoV, and a variety of SARS-related viruses. As compared with the traditional aluminum-adjuvanted vaccine against SARS-CoV-2, the new STING small molecule agonist can significantly improve the cellular and humoral immune responses of the vaccine, enhance the broad-spectrum protection against SARS and other coronaviruses, as well as significantly improve the persistence of the immunoprotection of the vaccine against SARS-CoV-2.

In one aspect, the present application provides a compound having formula (I) or pharmaceutically acceptable salts thereof,

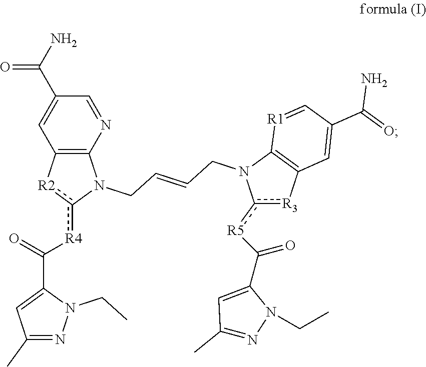

wherein R₁ is CR₁′, wherein R₁′ is H, —OMe or —O(CH₂)ₙNR₂′R₃′, n is an integer of 1 to 6, preferably 2 or 3, R₂′ and R₃′ taken together with the nitrogen atom through which they are connected to form a substituted or unsubstituted 5-6 membered heterocycloalkyl or substituted or unsubstituted 5-6 membered heteroaryl, wherein the substituted 5-6 membered heterocycloalkyl or substituted 5-6 membered heteroaryl is independently substituted with one or more halogen, OH, amine, CN, CF₃, or unsubstituted C₁-C₄ saturated alkyl; preferably, the heterocycloalkyl is one of:

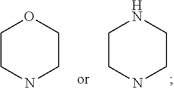

wherein R₂ and R₃ each independently are N or NR₄′, wherein R₄′ is H or C₁₋₄ saturated alkyl; and
wherein R₄ and R₅ each independently are N or NH.

In one embodiment, the compound has the structure:

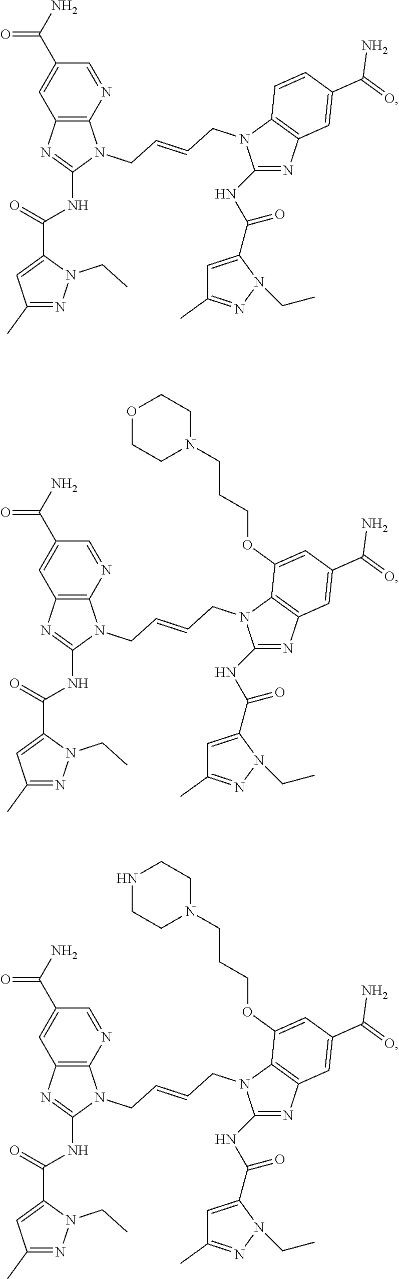 [SMILES: CCn1nc(C)cc1C(=O)Nc1nc2cc(C(N)=O)ccc2n1C/C=C/Cn1c(NC(=O)c2cc(C)nn2CC)nc2cc(C(N)=O)cnc21 ; CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN3CCOCC3)c21 ; CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN3CCNCC3)c21]

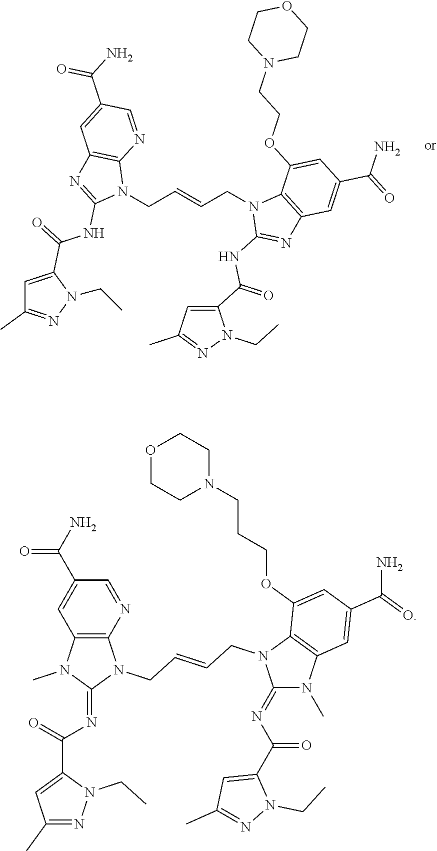 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCN3CCOCC3)c21 ; "or" ; CCn1nc(C)cc1C(=O)/N=c1\n(C)c2cc(C(N)=O)cnc2n1C/C=C/Cn1/c(=N/C(=O)c2cc(C)nn2CC)n(C)c2cc(C(N)=O)cc(OCCCN3CCOCC3)c21]

In one aspect, the application provides a pharmaceutical composition, comprising the compound of the application or pharmaceutically acceptable salts thereof, and at least one of a pharmaceutically acceptable carrier, a pharmaceutically acceptable excipient, and a pharmaceutically acceptable diluent.

In one aspect, the application provides use of the compound of the present application or pharmaceutically acceptable salts thereof or the pharmaceutical composition of the present application for the manufacture of an adjuvant. In one embodiment, the adjuvant is an adjuvant for a vaccine.

In one embodiment, the vaccine comprises an antigen. In one embodiment, the antigen is selected from a group consisting of a cancer antigen, a viral antigen, a bacterial antigen, a parasitic antigen, and a fungal antigen. In one embodiment, the vaccine is effective for preventing the infection of any one of the strains in the examples.

In one embodiment, the viral antigen is selected from a group consisting of an HIV antigen, an influenza antigen, and a coronavirus antigen. In one embodiment, the vial antigen is from one or more of HCoV-229E, HCoV-OC43, SARS-CoV, HCoV-NL63, HCoV-HKU1, MERS-CoV, Varicella zoster virus (VZV) and SARS-CoV-2 such as SARS-CoV-2 Omicron mutant. In one embodiment, the vial antigen is SARS-CoV-2 RBD-Fc protein or gE protein of Varicella zoster virus.

In one aspect, the application provides a vaccine, comprising the compound of the present application or pharmaceutically acceptable salts thereof; and an antigen. In one embodiment, the vaccine is an intramuscular, an intradermal vaccine, or an inhaled vaccine. In one embodiment, the vaccine comprises an antigen. In one embodiment, the antigen is selected from a group consisting of a cancer antigen, a viral antigen, a bacterial antigen, a parasitic antigen, and a fungi antigen. In one embodiment, the viral antigen is selected from a group consisting of an HIV antigen, an influenza antigen and a coronavirus antigen. In one embodiment, an antigen is from one or more of HCOV-229E, HCOV-OC43, SARS-COV, HCOV-NL63, HCOV-HKU1, MERS-COV, Varicella zoster virus (VZV) and SARS-COV-2 such as SARS-CoV-2 Omicron mutant. In one embodiment, the vial antigen is SARS-CoV-2 RBD-Fc protein or gE protein of Varicella zoster virus (VZV).

In one aspect, the application provides a method for producing a vaccine of the present application, comprising mixing the compound of the present application and an antigen.

In one aspect, the application provides the compound of the present application or pharmaceutically acceptable salts thereof for use as an adjuvant. In one embodiment, the adjuvant is an adjuvant for a vaccine. In one embodiment, the vaccine comprises an antigen. In one embodiment, the antigen is selected from a group consisting of a cancer antigen, a viral antigen, a bacterial antigen, a parasitic antigen, and a fungi antigen. In one embodiment, the viral antigen is selected from a group consisting of an HIV antigen, an influenza antigen and a coronavirus antigen. In one embodiment, an antigen is from one or more of HCOV-229E, HCOV-OC43, SARS-COV, HCOV-NL63, HCOV-HKU1, MERS-COV, Varicella zoster virus (VZV) and SARS-COV-2 such as SARS-CoV-2 Omicron mutant. In one embodiment, the vial antigen is SARS-CoV-2 RBD-Fc protein or gE protein of Varicella zoster virus.

In one aspect, the application provides a method for treating or preventing an infectious disease or a cancer, which comprises administering an effective amount of the vaccine of the present application to a subject in need thereof. In one embodiment, the vaccine is an intramuscular, intradermal vaccine or inhaled vaccine. In one embodiment, the infectious disease is selected from a group consisting of AIDS, severe acute respiratory syndrome (SARS), Middle East respiratory syndrome (MERS), COVID-19, Varicella zoster and influenza, and the cancer is selected from a group consisting of HPV-related cancer, HBV-related cancer, ovarian cancer, prostate cancer, breast cancer, brain cancer, head and neck cancer, laryngeal cancer, lung cancer, liver cancer, pancreatic cancer, kidney cancer, bone cancer, melanoma, metastatic cancer, HTERT-related cancer, FAP antigen-related cancer, non-small cell lung cancer, blood cancer, esophageal squamous cell carcinoma, cervical cancer, bladder cancer, colorectal cancer, gastric cancer, anal cancer, synovial sarcoma, testicular cancer, recurrent respiratory system papillomatosis, skin cancer, glioblastoma, liver cancer, gastric cancer, acute myeloid leukemia, triple-negative breast cancer, and primary cutaneous T-cell lymphoma.

In one aspect, the application provides a kit comprising the compound of the application, an antigen, and instructions for treating or preventing an infectious disease or a cancer.

The benefits of the present disclosure include:
(1) The application provides a new series of compound that can improve the immune responses to an antigen.
(2) the compounds in the present application, as adjuvants for the protein subunit RBD vaccine against SARS-CoV-2, can strongly activate the cellular and humoral immune responses in animal models, including mouse, rabbit and rhesus monkey, and produce a broad-spectrum, strong effective and long-lasting immunoprotection against SARS-CoV-2, SARS-CoV-2 mutants, SARS-CoV, and a variety of SARS-related viruses.
(3) Compared with the traditional aluminum adjuvant applied to the COVID-19 vaccine, the compounds of the present application can significantly improve the cellular and humoral immune responses to the vaccine, enhance the broad-spectrum protection against coronaviruses such as SARS, and significantly improve the persistence of the immunoprotection of the COVID-19 vaccine.
(4) The compound of the present application can effectively enhance the immune response to the polypeptide antigen of HIV.
(5) The compound of the present application can enhance the immune response to an influenza virus vaccine.

### DESCRIPTION OF THE FIGURES

FIG. 1 shows the results of the activation levels of various cytokines in the lymph nodes of mice at 6 hrs after the vaccination of mice with the STING agonist CF501 or cGAMP.

FIG. 2 shows the results of activation of the mouse cytokine IFNb at 6 hrs, 24 hrs and 48 hrs after the vaccination of mice with the STING agonist CF501 and a SARS-CoV-2 RBD-Fc protein.

FIG. 3 shows the results of activation of the mouse cytokine CXCL-10 at 6 hrs, 24 hrs and 48 hrs after the vaccination of mice with the STING agonist CF501 and a SARS-CoV-2 RBD-Fc protein.

FIG. 4 shows the results of activation of the mouse cytokine CCL-2 at 6 hrs, 24 hrs, and 48 hrs after the vaccination of mice with the STING agonist CF501 and a SARS-CoV-2 RBD-Fc protein.

FIG. 5 shows the results of activation of the mouse cytokine CXCL-9 at 6 hrs, 24 hrs, and 48 hrs after the vaccination of mice with the STING agonist CF501 and a SARS-CoV-2 RBD-Fc protein.

FIG. 6 shows the results of activation of the mouse cytokine IL-1b at 6 hrs, 24 hrs, and 48 hrs after the vaccination of mice with the STING agonist CF501 and a SARS-CoV-2 RBD-Fc protein.

FIG. 7 shows the results of activation of the mouse cytokine IL-6 at 6 hrs, 24 hrs, and 48 hrs after the vaccination of mice with the STING agonist CF501 and a SARS-CoV-2 RBD-Fc protein.

FIG. 8 shows the results of activation of the mouse cytokine TNF-α, at 6 hrs, 24 hrs, and 48 hrs after the vaccination of mice with the STING agonist CF501 and a SARS-CoV-2 RBD-Fc protein.

FIG. 9 shows the procedure for the vaccination of mice.

FIG. 10 shows the results of SARS-CoV-2 RBD specific IgG antibody titers at 21 days after the vaccination of mice in each group.

FIG. 11 shows the results of SARS-CoV-2 RBD specific IgG1 antibody titers at 21 days after the vaccination of mice in each group.

FIG. 12 shows the results of SARS-CoV-2 RBD specific IgG2a antibody titers at 21 days after the vaccination of mice in each group.

FIG. 13 shows the results of SARS-CoV-2 RBD specific IgG antibody titers at 35 days after the vaccination of mice in each group.

FIG. 14 shows the results of SARS-CoV-2 RBD specific IgG1 antibody titers at 35 days after the vaccination of mice in each group.

FIG. 15 shows the results of SARS-CoV-2 RBD specific IgG2a antibody titers at 35 days after the vaccination of mice in each group.

FIG. 16 shows the results of using the ELISPOT assay to detect the secretion of IFN-γ from the spleen of mice in each group at 35 days after the vaccination.

FIG. 17 shows the results of using the ELISPOT assay to detect the secretion of IFN-γ from the lungs of mice in each group at 35 days after the vaccination.

FIG. 18 shows the results of using the ELISPOT assay to detect the secretion of TNF-α from the spleen of mice in each group at 35 days after the vaccination.

FIG. 19 shows the results of using the ELISPOT assay to detect the secretion of TNF-α from the lungs of mice in each group at 35 days after the vaccination.

FIG. 20 shows the results of using the ELISPOT assay to detect the secretion of IL-4 from the spleen of mice in each group at 35 days after the vaccination.

FIG. 21 shows the results of using the ELISPOT assay to detect the secretion of IL-4 from the lungs of mice in each group at 35 days after the vaccination.

FIG. 22 shows the results of the neutralization titer against the SARS-CoV-2 pseudovirus in each group of mice at Day 21.

FIG. 23 shows the results of the neutralization titer against the SARS-CoV-2 pseudovirus in each group of mice at Day 35.

FIG. 24 shows the results of the correlation between RBD-specific antibodies and neutralizing antibodies in the sera of each group of mice at Day 21.

FIG. 25 shows the results of the correlation between RBD-specific antibodies and neutralizing antibodies in the sera of each group of mice at Day 35.

FIG. 26 shows the results of using the plaque reduction assay to detect the neutralization activity against the SARS-CoV-2 live virus in each group of mice at Day 21.

FIG. 27 shows the results of using the plaque reduction assay to detect the neutralization activity against the SARS-CoV-2 live virus in each group of mice at Day 35.

FIG. 28 shows the results of using the immunofluorescence assay to detect the neutralization activity against the SARS-CoV-2 live virus in each group of mice at Day 21.

FIG. 29 shows the results of using the immunofluorescence assay to detect the neutralization activity against the SARS-CoV-2 live virus in each group of mice at Day 35.

FIG. 30 shows that the sera of mice vaccinated with the CF501 and RBD-Fc can inhibit SARS-CoV-2 mediated membrane fusion.

FIG. 31 shows the results of SARS-CoV RBD specific antibody titers in the sera of each group of mice at Day 35.

FIG. 32 shows the results of the neutralization activity against the SARS-CoV pseudovirus in each group of mice at Day 35.

FIG. 33 shows the correlation between the neutralizing antibodies against the SARS-CoV pseudovirus and SARS-CoV RBD specific antibodies in each group of mice at Day 35.

FIG. 34 shows the results of the neutralization activity against the WIV1 pseudovirus in each group of mice at Day 35.

FIG. 35 shows the results of the neutralization activity against the Rs3367 pseudovirus in each group of mice at Day 35.

FIG. 36 shows the results of changes in the body weight of the mice after the vaccinated mice were challenged.

FIG. 37 shows the results of viral load in the lungs of mice at Day 4 after the vaccinated mice were challenged.

FIG. 38 shows the results of viral load in the brains of mice at Day 4 after the vaccinated mice were challenged.

FIG. 39 shows the results of viral load in the intestines of mice at Day 4 after the vaccinated mice were challenged.

FIG. 40 shows the procedure for the vaccination of New Zealand white rabbits.

FIG. 41 shows the results of SARS-CoV-2 RBD specific IgG antibody titers at 21 days after the vaccination of New Zealand white rabbits in each group.

FIG. 42 shows the results of SARS-CoV-2 RBD specific IgG antibody titers at 35 days after the vaccination of New Zealand white rabbits in each group.

FIG. 43 shows the results of the neutralization activity of the sera against the SARS-CoV-2 pseudovirus at 21 days after the vaccination of New Zealand white rabbits in each group.

FIG. 44 shows the results of the neutralization activity of the sera against the SARS-CoV-2 pseudovirus at 35 days after the vaccination of New Zealand white rabbits in each group.

FIG. 45 shows the results of the correlation between neutralizing antibodies and SARS-CoV-2 RBD specific antibodies in the sera at 21 days after the vaccination of New Zealand white rabbits in each group.

FIG. 46 shows the results of the correlation between neutralizing antibodies and SARS-CoV-2 RBD specific antibodies in the sera at 35 days after the vaccination of New Zealand white rabbits in each group.

FIG. 47 shows the results of using the plaque reduction method to detect the neutralization activity of the sera against the SARS-CoV-2 live virus at 21 days after the vaccination of New Zealand white rabbits in each group.

FIG. 48 shows the results of using the plaque reduction method to detect the neutralization activity of the sera against the SARS-CoV-2 live virus at 35 days after the vaccination of New Zealand white rabbits in each group.

FIG. 49 shows the results of using the immunofluorescence assay to detect the neutralization activity of the sera against the SARS-CoV-2 live virus at 21 days after the vaccination of New Zealand white rabbits in each group.

FIG. 50 shows the results of using the immunofluorescence assay to detect the neutralization activity of the sera against the SARS-CoV-2 live virus at 35 days after the vaccination of New Zealand white rabbits in each group.

FIG. 51 shows that the sera of New Zealand white rabbits vaccinated with the CF501 and RBD-Fc can inhibit SARS-CoV-2 mediated membrane fusion.

FIG. 52 shows the results of the neutralization activity of the sera against the SARS-CoV-2 pseudovirus at 49 days after the vaccination of New Zealand white rabbits in each group.

FIG. 53 shows the results of using the plaque reduction method to detect the neutralization activity of the sera against the SARS-CoV-2 live virus at 49 days after the vaccination of New Zealand white rabbits in each group.

FIG. 54 shows the results of the neutralization activity of the sera against the SARS-CoV pseudovirus at 35 days after the vaccination of New Zealand white rabbits in each group.

FIG. 55 shows the results of the neutralization activity of the sera against the WIV1 pseudovirus at 35 days after the vaccination of New Zealand white rabbits in each group.

FIG. 56 shows the results of SARS-CoV RBD specific IgG antibody titers at 49 days after the vaccination of New Zealand white rabbits in each group.

FIG. 57 shows the results of the neutralization activity against the SARS-CoV pseudovirus at 49 days after the vaccination of New Zealand white rabbits in each group.

FIG. 58 shows the results of the correlation between SARS-CoV RBD specific antibodies and neutralizing antibodies against SARS-CoV pseudovirus in sera at 49 days after the vaccination of New Zealand white rabbits in each group.

FIG. 59 shows the results of the neutralization activity against WIV1 pseudovirus at 49 days after the vaccination of New Zealand white rabbits in each group.

FIG. 60 shows the results of the neutralization activity against Rs3367 pseudovirus at 49 days after the vaccination of New Zealand white rabbits in each group.

FIG. 61 shows the results of the neutralization activity against pseudoviruses of various SARS-CoV-2 mutants at 49 days after the vaccination of New Zealand white rabbits in each group.

FIG. 62 shows the procedure for the vaccination of rhesus monkeys.

FIG. 63 shows the results of the IgG antibody bound to the SARS-CoV-2 RBD in the sera at 14 days after the vaccination of rhesus monkeys in each group.

FIG. 64 shows the IgG antibody titers against SARS-CoV-2 RBD in the sera at 14 days after the vaccination of rhesus monkeys in each group.

FIG. 65 shows the results of the IgG antibody bound to SARS-CoV-2 RBD in the sera at 28 days after the vaccination of rhesus monkeys in each group.

FIG. 66 shows the IgG antibody titers against SARS-CoV-2 RBD in the sera at 28 days after the vaccination of rhesus monkeys in each group.

FIG. 67 shows the results of the level of IFN-γ secretion by PBMC (peripheral blood mononuclear cells) at 14 days after the vaccination of rhesus monkeys in each group.

FIG. 68 shows the results of the level of IFN-γ secretion by PBMC (peripheral blood mononuclear cells) at 28 days after the vaccination of rhesus monkeys in each group.

FIG. 69 shows the results of the inhibition of SARS-CoV-2 pseudovirus infection by the sera at 14 days after the vaccination of rhesus monkeys in each group.

FIG. 70 shows the results of the inhibition of SARS-CoV-2 pseudovirus infection by the sera at 28 days after the vaccination of rhesus monkeys in each group.

FIG. 71 shows the results of the correlation between the SARS-CoV-2 RBD specific antibody titers and the neutralizing antibody titers in the sera at 28 days after the vaccination of rhesus monkeys in each group.

FIG. 72 shows the results of using the plaque reduction method to detect the inhibition of the infection of the SARS-CoV-2 live virus by the sera at 28 days after the vaccination of rhesus monkeys in each group.

FIG. 73 shows the results of using the plaque reduction method to detect the SARS-CoV-2 live virus-neutralizing antibody titer in the sera at different time points from 14 days to 191 days after the vaccination of rhesus monkeys.

FIG. 74 shows the neutralization activities of the sera from each group of the rhesus monkeys against pseudoviruses of SARS-CoV-2 variants and single-point mutants.

FIG. 75 shows the IgG endpoint titers of the antibodies specific to RBD of the Omicron strain in serum at 28 days to 191 days after vaccination of rhesus monkeys in each group.

FIG. 76 shows the neutralizing antibody titer of the sera against the Omicron pseudovirus at 28 days to 191 days after vaccination of rhesus monkeys in each group.

FIG. 77 shows that the sera of rhesus monkeys at 122 days after vaccination show neutralization activity against the live Omicron virus.

FIG. 78 shows the results of the inhibition of the infection of the SARS-CoV pseudovirus by the sera at 28 days after the vaccination of rhesus monkeys in each group.

FIG. 79 shows the results of the correlation between SARS-CoV-2 RBD specific antibody titers and the SARS-CoV neutralizing antibody titers in the sera at 28 days after the vaccination of rhesus monkeys in each group.

FIG. 80 shows the results of the inhibition of the infection of the WIV1 pseudovirus by the sera at 28 days after the vaccination of rhesus monkeys in each group.

FIG. 81 shows the results of the correlation between the SARS-CoV-2 RBD specific antibody titers and the WIV1 neutralizing antibody titers in the sera at 28 days after the vaccination of rhesus monkeys in each group.

FIG. 82 shows the results of the inhibition of the infection of the Rs3367 pseudovirus by the sera at 28 days after the vaccination of rhesus monkeys in each group.

FIG. 83 shows the results of the correlation between the SARS-CoV-2 RBD specific antibody titers and the Rs3367 neutralizing antibody titers in the sera at 28 days after the vaccination of rhesus monkeys in each group.

FIG. 84 shows SARS-CoV-2 viral loads in the nasal swabs at 3, 5, and 7 days post-challenge for the rhesus macaques treated with PBS.

FIG. 85 shows SARS-CoV-2 viral loads in the nasal swabs at 3, 5, and 7 days post-challenge for the rhesus macaques immunized with Alum/RBD-Fc.

FIG. 86 shows SARS-CoV-2 viral loads in the nasal swabs at 3, 5, and 7 days post-challenge for the rhesus macaques immunized with CF501/RBD-Fc.

FIG. 87 shows SARS-CoV-2 viral loads in the indicated lung lobes from the immunized macaques at 7 days post-challenge.

FIG. 88 shows SARS-CoV-2 viral loads in the nasal turbinate from the immunized macaques at 7 days post-challenge.

FIG. 89 shows SARS-CoV-2 viral loads in the nasal mucosa from the immunized macaques at 7 days post-challenge.

FIG. 90 shows the results of N3G protein-specific antibodies in the sera of mice vaccinated with the CF501 and the HIV N3G protein or only with the HIV N3G protein.

FIG. 91 shows the results of H1N1 HA specific antibodies in the sera at Day 14 after the vaccination of mice with the influenza virus quadrivalent vaccine with different adjuvants.

FIG. 92 shows the results of H3N2 HA specific antibodies in the sera at Day 14 after the vaccination of mice with the influenza virus quadrivalent vaccine with different adjuvants.

FIG. 93 shows the results of B/Victoria HA specific antibodies in the sera at Day 14 after the vaccination of mice with the influenza virus quadrivalent vaccine with different adjuvants.

FIG. 94 shows the results of B/Yamagata HA specific antibodies in the sera at Day 14 after the vaccination of mice with the influenza virus quadrivalent vaccine with different adjuvants.

FIG. 95 shows the results of H1N1 HA specific antibodies in the sera at Day 21 after the vaccination of mice with the influenza virus quadrivalent vaccine with different adjuvants.

FIG. 96 shows the results of H3N2 HA specific antibodies in the sera at Day 21 after the vaccination of mice with the influenza virus quadrivalent vaccine with different adjuvants.

FIG. 97 shows the results of B/Victoria HA specific antibodies in the sera at Day 21 after the vaccination of mice with the influenza virus quadrivalent vaccine with different adjuvants.

FIG. 98 shows the results of B/Yamagata HA specific antibodies in the sera at Day 21 after the vaccination of mice with the influenza virus quadrivalent vaccine with different adjuvants.

FIG. 99 shows the detection of H1N1 HA specific antibodies in the sera at Days 14 and 21 after the vaccination of mice with the influenza virus quadrivalent vaccine with different adjuvants.

FIG. 100 shows the detection of H3N2 HA specific antibodies in the sera at Days 14 and 21 after the vaccination of mice with the influenza virus quadrivalent vaccine with different adjuvants.

FIG. 101 shows the detection of B/Victoria HA specific antibodies in the sera at Days 14 and 21 after the vaccination of mice with the influenza virus quadrivalent vaccine with different adjuvants.

FIG. 102 shows the detection of B/Yamagata HA specific antibodies in the sera at Days 14 and 21 after the vaccination of mice with the influenza virus quadrivalent vaccine with different adjuvants.

FIG. 103 shows the detection of varicella-zoster virus gE protein-specific antibodies in the sera from mice immunized with varicella-zoster virus inactivated vaccines plus different adjuvants at 21 days post the first immunization.

FIG. 104 shows the detection of varicella-zoster virus gE protein-specific antibodies in the sera from mice immunized with varicella-zoster virus inactivated vaccines plus different adjuvants at 35 days post the first immunization.

### DETAILED DESCRIPTION OF INVENTION

The terms “halo” or “halogen” refers to fluorine, chlorine, bromine or iodine.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di- and multivalent radicals. The alkyl may include a designated number of carbons (e.g., C₁-C₁₀ means one to ten carbons). In embodiments, the alkyl is fully saturated. In embodiments, the alkyl is monounsaturated. In embodiments, the alkyl is polyunsaturated. Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs, and isomers of, for example, n-pentyl, n-hexyl, n-heptyl, n-octyl, and the like. In embodiments, the term “alkyl” refers to a straight or branched hydrocarbon chain group consisting solely of carbon and hydrogen atoms, containing no unsaturation (i.e., saturated alkyl); having from one to ten, one to eight, one to six, or one to four carbon atoms; and which is attached to the rest of the molecule by a single bond, e.g., methyl, ethyl, n-propyl, 1-methylethyl (iso-propyl), n-butyl, n-pentyl, 1, 1-dimethylethyl (t-butyl), and the like.

In embodiments, the term “heterocycloalkyl” means a monocyclic, bicyclic, or a multicyclic heterocycloalkyl ring system. In embodiments, heterocycloalkyl groups are fully saturated. In embodiments, a bicyclic or multicyclic heterocycloalkyl ring system refers to multiple rings fused together wherein at least one of the fused rings is a heterocycloalkyl ring and wherein the multiple rings are attached to the parent molecular moiety through any atom contained within a heterocycloalkyl ring of the multiple rings.

In embodiments, a heterocycloalkyl is a “heterocyclyl”, which refers to a stable 3- to 15-membered ring group which consists of carbon atoms and from one to five heteroatoms selected from a group consisting of nitrogen, oxygen and sulfur. In one embodiment, the heterocyclic ring system group may be a monocyclic, bicyclic, or tricyclic ring or tetracyclic ring system, which may include fused or bridged ring systems; and the nitrogen or sulfur atoms in the heterocyclic ring system group may be optionally oxidized; the nitrogen atom may be optionally quaternized; and the heterocyclyl group may be partially or fully saturated or aromatic. The heterocyclic ring system may be attached to the main structure at any heteroatom or carbon atom which results in the creation of a stable compound. Exemplary heterocylic radicals include, azetidinyl, benzopyranonyl, benzopyranyl, benzotetrahydrofuranyl, benzotetrahydrothienyl, chromanyl, chromonyl, coumarinyl, decahydroisoquinolinyl, dibenzofuranyl, dihydrobenzisothiazinyl, dihydrobenzisoxazinyl, dihydrofuryl, dihydropyranyl, dioxolanyl, dihydropyrazinyl, dihydropyridinyl, dihydropyrazolyl, dihydropyrimidinyl, dihydropyrrolyl, dioxolanyl, 1,4 dithianyl, isobenzotetrahydrofuranyl, isobenzotetrahydrothienyl, isochromanyl, isocoumarinyl, benzo[1,3]dioxol-5-yl, benzodioxolyl, 1,3-dioxolan-2-yl, dioxolanyl, morpholinyl, octahydroindolyl, octahydroisoindolyl, tetrahydrofuran, oxazolidin-2-onyl, oxazolidinonyl, piperidinyl, piperazinyl, pyranyl, tetrahydroiuryl, tetrahydrofuranyl, tetrahydroisoquinolinyl, tetrahydropyranyl, tetrahydrothienyl, pyrrolidinonyl, oxathiolanyl, and pyrrolidinyl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. In embodiments, a fused ring aryl refers to multiple rings fused together wherein at least one of the fused rings is an aryl ring and wherein the multiple rings are attached to the parent molecular moiety through any carbon atom contained within an aryl ring of the multiple rings. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. In embodiments, the term “heteroaryl” includes fused ring heteroaryl groups (i.e., multiple rings fused together wherein at least one of the fused rings is a heteroaromatic ring and wherein the multiple rings are attached to the parent molecular moiety through any atom contained within a heteroaromatic ring of the multiple rings). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. A 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl.

As used herein, the symbol “ [symbol] ” means that two atoms may be linked via a single bond or a double bond, provided that the valence states of the two atoms is suitable.

The term “antigen” refers to any substance that can be used as a target of an immune response. The immune response can be a cellular immune response or a body fluid immune response. In one embodiment, a vaccine comprises an antigen. In one embodiment, the antigen is selected from the group consisting of a cancer antigen, a viral antigen, a bacterial antigen, a parasitic antigen, and a fungi antigen.

In one embodiment, the viral antigen is selected from a group consisting of an HIV antigen, an influenza antigen, and a coronavirus antigen. In one embodiment, the viral antigen is an antigen from one or more of HCOV-229E, HCOV-OC43, SARS-COV, HCOV-NL63, HCOV-HKU1, MERS-COV, Varicella zoster virus and SARS-COV-2 such as SARS-CoV-2 Omicron mutant. In one embodiment, the viral antigen is a SARS-COV-2 RBD-Fc protein or gE protein of Varicella zoster virus (VZV).

In one aspect, the present disclosure provides a vaccine comprising the compound of the disclosure and an antigen. In one embodiment, the vaccine is an intramuscular, an intracellular vaccine or an inhaled vaccine. The antigen can be a cancer antigen, a viral antigen, a bacterial antigen, a parasitic antigen, and/or a fungi antigen. For example, the viral antigen can be selected from a group consisting of an HIV antigen, an influenza antigen and a coronavirus antigen. The viral antigen can be an antigen from one or more of HCOV-229E, HCOV-OC43, SARS-COV, HCOV-NL63, HCOV-HKU1, MERS-COV, Varicella zoster virus and and SARS-COV-2 such as SARS-CoV-2 Omicron mutant. In one embodiment, the viral antigen is a SARS-COV-2 RBD-Fc protein or gE protein of Varicella zoster virus (VZV).

The term “vaccine” herein refers to a formulation of an antigen, which typically contains certain portions of infective sources and raises immune response in a subject after its injection. The antigenic portion of the vaccine formulation can be a microorganism or a natural product purified from a microorganism, a synthetic product or a genetic engineering protein, a peptide, a polysaccharide etc. Preferably, the vaccine is an inactivated vaccine, live-attenuated vaccine, subunit vaccine, nucleic acid vaccine such as mRNA or DNA vaccine.

The term “adjuvant” as used herein refers to any substance that can increase or modify an immune response after mixing with the injected immunogen. Herein, the adjuvant can be one or more compounds as shown below:

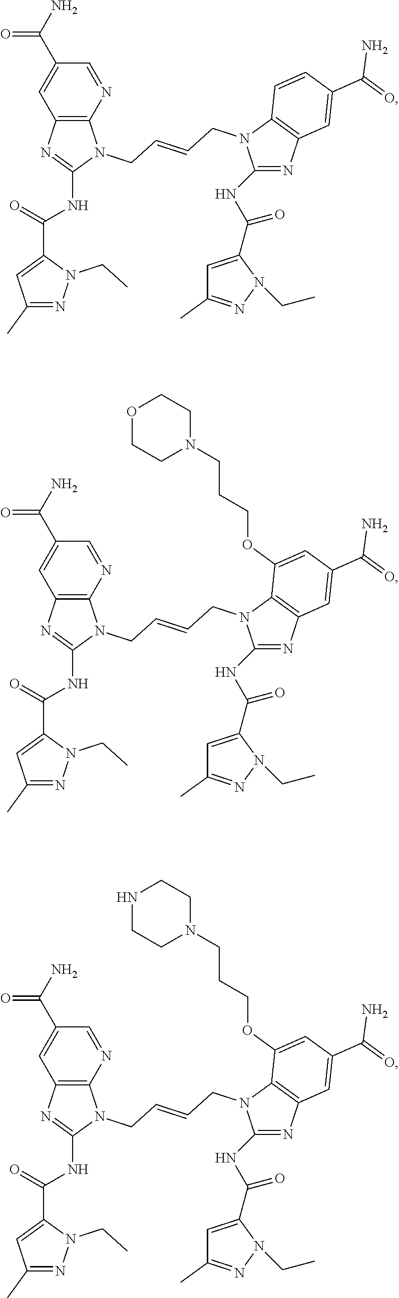 [SMILES: CCn1nc(C)cc1C(=O)Nc1nc2cc(C(N)=O)ccc2n1C/C=C/Cn1c(NC(=O)c2cc(C)nn2CC)nc2cc(C(N)=O)cnc21 ; CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN3CCOCC3)c21 ; CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN3CCNCC3)c21]

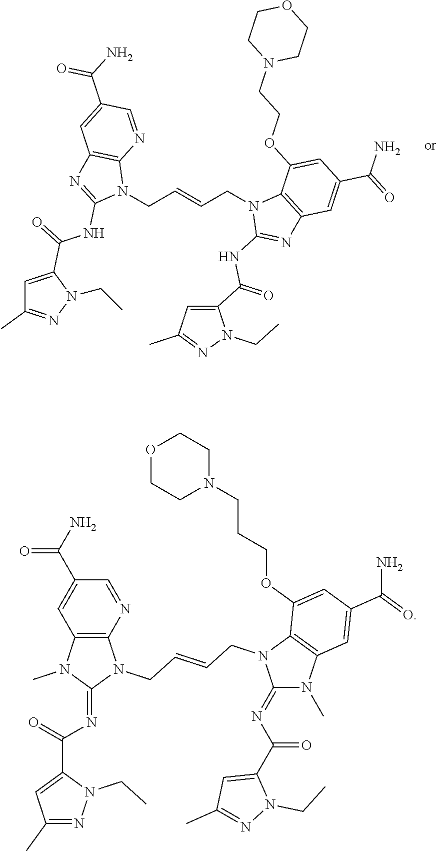 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCN3CCOCC3)c21 ; "or" ; CCn1nc(C)cc1C(=O)/N=c1\n(C)c2cc(C(N)=O)cnc2n1C/C=C/Cn1/c(=N/C(=O)c2cc(C)nn2CC)n(C)c2cc(C(N)=O)cc(OCCCN3CCOCC3)c21]

Effective vaccines must cause appropriate responses to antigens. There are several unique types of immune responses that have different protection capabilities to resist specific diseases. For example, antibodies have a protective effect against bacterial infections, but a cell-mediated immunity is required to remove many viral infections and tumors. There are a variety of unique antibodies and cell-mediated immune response. The cell-mediated response is divided into two basic groups: 1) delayed hypersensitivity reaction, where T cells function indirectly through macrophages and other cells or cell products, and 2) cytotoxic reactions, wherein specialized T cells specifically and directly attack and kill infected cells.

There are five primary antibodies: IgM, IgG, IgE, IgA, and IgD. These antibodies have unique functions of immune responses. IgG, a type of antibody that is dominant in the blood, can be subdivided into several different subclasses or isotypes. In mice, the isotypes are IgG1, IgG2a, IgG2b, and IgG3. In human, the isotypes are IgG1, IgG2, IgG3, and IgG4. The IgG isotype has different protection capabilities against specific infections. The murine IgG2a and IgG2b can activate complements and mediate the antibody-mediated cytotoxicity and the cell-mediated cytotoxicity. They are particularly effective against many bacterial, viral and parasitic infections. The similar isotypes in human are represented by IgG1 and IgG3. In contrast, the murine IgG3 has a particularly effective protection against bacteria with a polysaccharide film, such as pneumococcus. The isotype in human may be IgG4. The isotypes such as murine IgG1 do not bind to a complement, and cannot effectively neutralize a toxin. Their effects on many bacterial and viral infections are low. Since different IgG isotypes have significantly different immune function, it is important to induce a most suitable isotype with a vaccine for a particular infection Although the names are different, the effective evidence and the prior theories have shown that the nature of an immunogen that determines the isotypes of antibodies among mammal species is similar. In other words, in a species, an immunogen which can invoke IgG antibodies in delayed type hypersensitivity or IgG antibodies in complement binding can stimulate similar responses in another species.

In some embodiments, the present application provides a kit comprising an immunogenic composition or a vaccine and instructions for use. The kit can contain immunogenic compositions or vaccines in a suitable container and various buffers well known in the art. In some embodiments, the kit comprises one or more compounds of the present application. Thus, in some embodiments, immunogenic compositions or vaccines and these compounds are in the same vial. In some embodiments, immunogenic compositions or vaccines and these compounds are in separate vials.

The container may include at least one vial, a tube, a flask, a bottle, a syringe, or other container device, which can contain an immunogenic composition or a vaccine. If other components are provided, the kit can contain other containers that hold the components. The kit can also include means for containing an immunogenic composition or a vaccine, and any other reagent container that is closed for commercial sales. Such containers can include injection or blow molding plastic containers that retain the desired vials therein. The container and/or kits can include labels with instructions and/or warnings. In some embodiments, the present application provides a kit comprising a container that includes a vaccine comprising an immunogenic composition, an optional pharmaceutically acceptable carrier, and packaging insert with instructions for the vaccination for the treatment or prevention of the disease in a subject. In some embodiments, the kit further comprises a compound of the present application and an instruction for administrating the compound for the treatment or prevention of the disease in a subject.

The present application is further illustrated by the following examples. The examples should not be construed as limiting.

### EXAMPLES

The following compounds are used in the examples and the synthesis of the compounds is as follows. The starting reagents are commercial available.

### (E)-3-(4-(5-carbamoyl-2-(1-ethyl-3-methyl-1H-pyrazole-5-carboxamido)-1H-benzo[d]imidazol-1-yl)but-2-en-1-yl)-2-(1-ethyl-3-methyl-1H-pyrazole-5-carboxamido)-3H-imidazo[4,5-b]pyridine-6-carboxamide (which is Referred as “STING Agonist 502” or CF502)

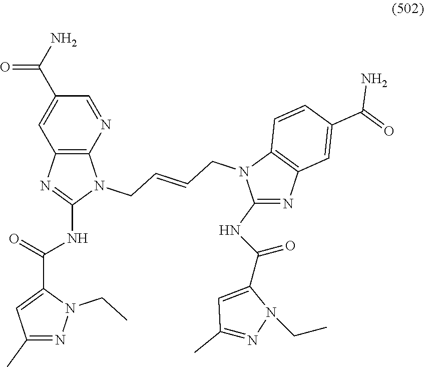 [SMILES: CCn1nc(C)cc1C(=O)Nc1nc2cc(C(N)=O)ccc2n1C/C=C/Cn1c(NC(=O)c2cc(C)nn2CC)nc2cc(C(N)=O)cnc21]

### (E)-3-(4-(5-carbamoyl-2-(1-ethyl-3-methyl-1H-pyrazole-5-carboxamido)-7-(3-morpholinopropoxy)-1H-benzo[d]imidazol-1-yl)but-2-en-1-yl)-2-(1-ethyl-3-methyl-1H-pyrazole-5-carboxamido)-3H-imidazo[4,5-b]pyridine-6-carboxamide (which is Referred as “STING Agonist 501” or CF501)

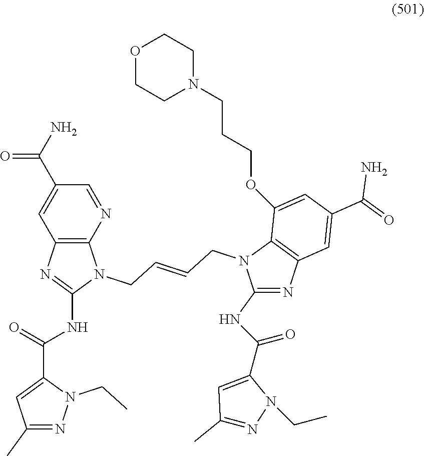 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN3CCOCC3)c21]

### (E)-3-(4-(5-carbamoyl-2-(1-ethyl-3-methyl-1H-pyrazole-5-carboxamido)-7-(3-(piperazin-1-yl)propoxy)-1H-benzo[d]imidazol-1-yl)but-2-en-1-yl)-2-(1-ethyl-3-methyl-1H-pyrazole-5-carboxamido)-3H-imidazo[4,5-b]pyridine-6-carboxamide (which is Referred as “STING Agonist 508” or CF508)

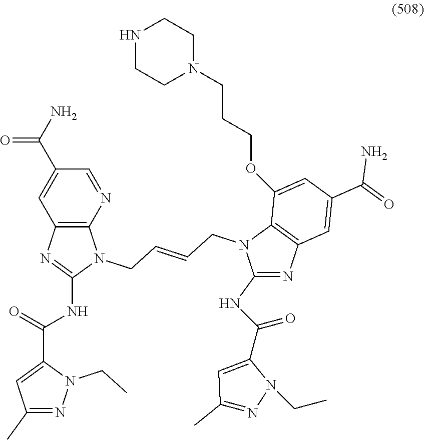 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN3CCNCC3)c21]

### (E)-3-(4-(5-carbamoyl-2-(1-ethyl-3-methyl-1H-pyrazole-5-carboxamido)-7-(2-morpholinoethoxy)-1H-benzo[d]imidazol-1-yl)but-2-en-1-yl)-2-(1-ethyl-3-methyl-1H-pyrazole-5-carboxamido)-3H-imidazo[4,5-b]pyridine-6-carboxamide (which is Referred as “STING Agonist 510” or CF510)

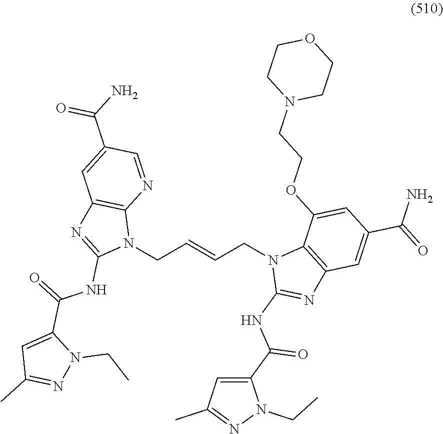 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCN3CCOCC3)c21]

### (E)-3-((E) 4-((E)-5-carbamoyl-2-((1-ethyl-3-methyl-1H-pyrazole-5-carbonyl)imino)-3-methyl-7-(3-morpholinopropoxy)-2,3-dihydro-1H-benzo[d]imidazol-1-yl)but-2-en-1-yl)-2-((1-ethyl-3-methyl-1H-pyrazole-5-carbonyl)imino)-1-methyl-2,3-dihydro-1H-imidazo[4,5-b]pyridine-6-carboxamide (which is Referred as “STING Agonist 512” or CF512)

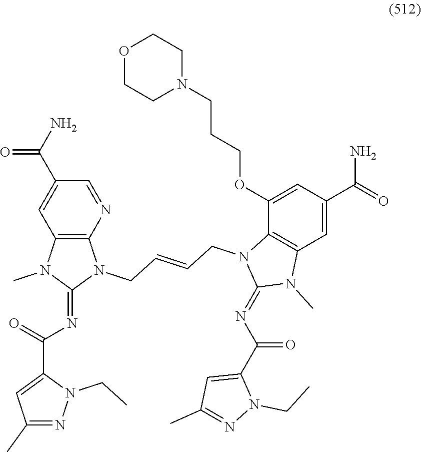 [SMILES: CCn1nc(C)cc1C(=O)/N=c1\n(C)c2cc(C(N)=O)cnc2n1C/C=C/Cn1/c(=N/C(=O)c2cc(C)nn2CC)n(C)c2cc(C(N)=O)cc(OCCCN3CCOCC3)c21]

### The Compound CF502 and its Synthesis

### (E)-3-(4-(5-carbamoyl-2-(1-ethyl-3-methyl-1H-pyrazole-5-carboxamido)-1H-benzo[d]imidazol-1-yl)but-2-en-1-yl)-2-(1-ethyl-3-methyl-1H-pyrazole-5-carboxamido)-3H-imidazo[4,5-b]pyridine-6-carboxamide

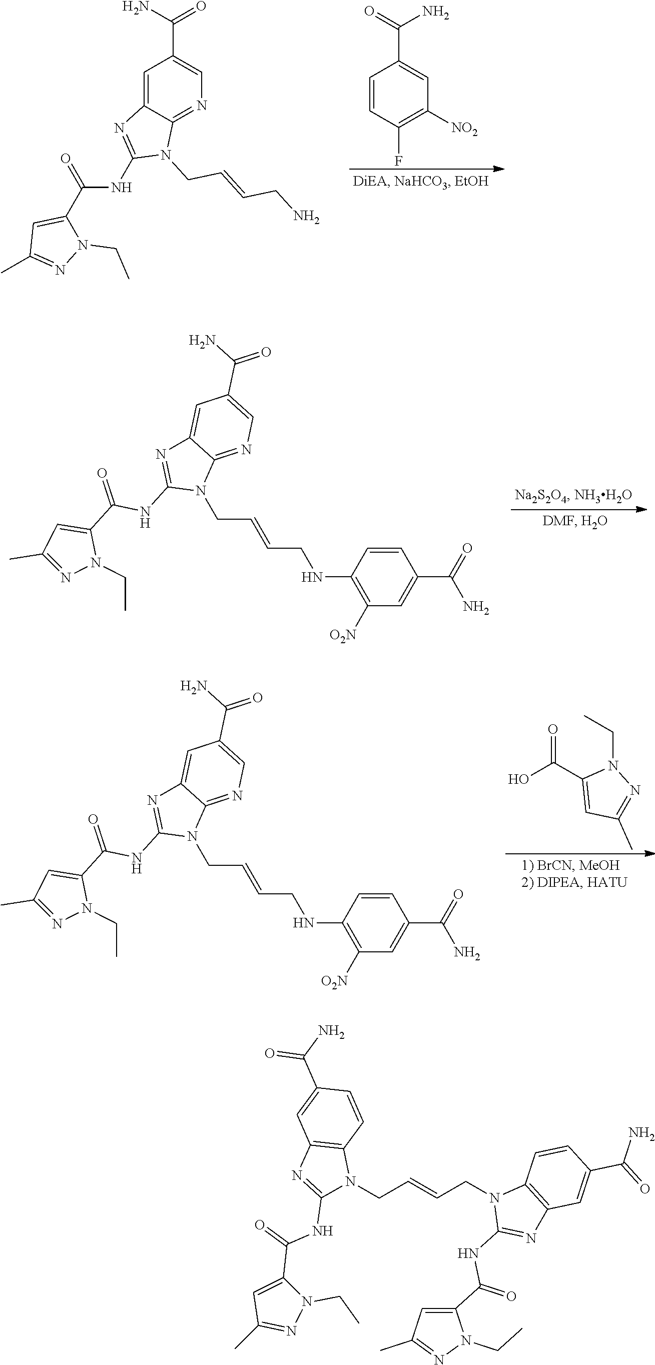 [SMILES: CCn1nc(C)cc1C(=O)Nc1nc2cc(C(N)=O)cnc2n1C/C=C/CN ; NC(=O)c1ccc(F)c([N+](=O)[O-])c1 ; CCn1nc(C)cc1C(=O)Nc1nc2cc(C(N)=O)cnc2n1C/C=C/CNc1ccc(C(N)=O)cc1[N+](=O)[O-] ; CCn1nc(C)cc1C(=O)Nc1nc2cc(C(N)=O)cnc2n1C/C=C/CNc1ccc(C(N)=O)cc1[N+](=O)[O-] ; CCn1nc(C)cc1C(=O)O ; CCn1nc(C)cc1C(=O)Nc1nc2cc(C(N)=O)ccc2n1C/C=C/Cn1c(NC(=O)c2cc(C)nn2CC)nc2cc(C(N)=O)ccc21]

### Scheme of Synthesis

### Step 1

### (E)-3-(4-((4-carbamoyl-2-nitrophenyl)amino)but-2-en-1-yl)-2-(1-ethyl-3-methyl-1H-pyrazole-5-carboxamido)-3H-imidazo[4,5-b]pyridine-6-carboxamide

To a solution of (E)-3-(4-aminobut-2-en-1-yl)-2-(1-ethyl-3-methyl-1H-pyrazole-5-carboxamido)-3H-imidazo[4,5-b]pyridine-6-carboxamide (Intermediate 2, 6.00 g, 14.342 mmol, 1.00 eq, HCl), 4-fluoro-3-nitro-benzamide (2.64 g, 14.3 mmol, 1.00 eq), DIPEA (7.40 g, 57.3 mmol, 9.98 mL, 4.00 eq) and NaHCO₃ (4.81 g, 57.30 mmol, 2.23 mL, 4 eq) in EtOH (60 mL) was stirred at 110° C. for 16 hrs under N₂ to afford a yellow suspension. LCMS showed one main peak with desired MS peak was found. The reaction mixture was diluted with H₂O (60 mL) and filtered to provide (E)-3-(4-((4-carbamoyl-2-nitrophenyl)amino)but-2-en-1-yl)-2-(1-ethyl-3-methyl-1H-pyrazole-5-carboxamido)-3H-imidazo[4,5-b]pyridine-6-carboxamide (5.0 g, 9.14 mmol, 63.80% yield, 99.9% purity) was obtained as yellow solid, which was used directly in the next step without further purification.

¹HNMR (400 MHz, DMSO-d₆): δ (ppm) 12.83 (br s, 1H), 8.72 (d, J=1.50 Hz, 1H), 8.62 (d, J=2.00 Hz, 1H), 8.49 (t, J=5.94 Hz, 1H), 8.13 (br s, 2H), 7.86-8.01 (m, 2H), 7.51 (br s, 1H), 7.25 (br s, 1H), 6.95 (d, J=9.13 Hz, 1H), 6.59 (s, 1H), 5.86-5.96 (m, 1H), 5.72-5.83 (m, 1H), 4.81 (br d, J=4.88 Hz, 2H), 4.55 (q, J=7.05 Hz, 2H), 4.05 (br s, 2H), 2.15 (s, 3H), 1.31 (t, J=7.07 Hz, 3H). LCMS: m/z 547.2 (M+1).

### Step 2

### (E)-3-(4-((2-amino-4-carbamoylphenyl)amino)but-2-en-1-yl)-2-(1-ethyl-3-methyl-1H-pyrazole-5-carboxamido)-3H-imidazo[4,5-b]pyridine-6-carboxamide

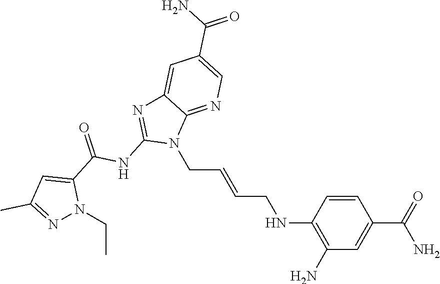 [SMILES: CCn1nc(C)cc1C(=O)Nc1nc2cc(C(N)=O)cnc2n1C/C=C/CNc1ccc(C(N)=O)cc1N]

To a solution of (E)-3-(4-((4-carbamoyl-2-nitrophenyl)amino)but-2-en-1-yl)-2-(1-ethyl-3-methyl-1H-pyrazole-5-carboxamido)-3H-imidazo[4,5-b]pyridine-6-carboxamide (5.00 g, 9.15 mmol, 1.00 eq) in DMF (50 mL) and H₂O (25 mL) was added NH₃·H₂O (12.8 g, 91.4 mmol, 14.0 mL, 25% purity, 10.0 eq) followed by disodium; BLAH (4.78 g, 27.4 mmol, 5.97 mL, 3.00 eq) and the reaction mixture was stirred at 25° C. for 1 hr to afford a yellow suspension. LCMS showed desired MS peak was found. The reaction mixture was diluted with H₂O (500 mL) and lyophilized. The residue was diluted with DMF (400 mL) and filtered. The filtrate was concentrated in vacuum. (E)-3-(4-((2-amino-4-carbamoylphenyl)amino)but-2-en-1-yl)-2-(1-ethyl-3-methyl-1H-pyrazole-5-carboxamido)-3H-imidazo[4,5-b]pyridine-4-carboxamide (4.5 g, crude) was obtained as light yellow solid, which was used directly in the next step without purification.

LCMS: m/z 517.3 (M+1).

### Step 3

### (E)-3-(4-(2-amino-5-carbamoyl-1H-benzo[d]imidazol-1-yl)but-2-en-1-yl)-2-(1-ethyl-3-methyl-1H-pyrazole-5-carboxamido)-3H-imidazo[4,5-b]pyridine-6-carboxamide

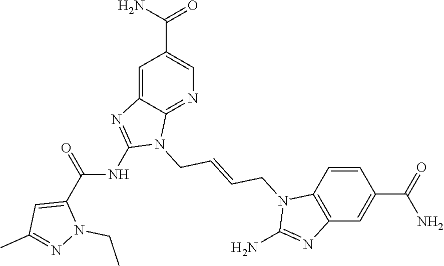 [SMILES: CCn1nc(C)cc1C(=O)Nc1nc2cc(C(N)=O)cnc2n1C/C=C/Cn1c(N)nc2cc(C(N)=O)ccc21]

To a solution of (E)-3-(4-((2-amino-4-carbamoylphenyl)amino)but-2-en-1-yl)-2-(1-ethyl-3-methyl-1H-pyrazole-5-carboxamido)-3H-imidazo[4,5-b]pyridine-6-carboxamide (4.5 g, 8.71 mmol, 1 eq) in DMF (45 mL) and MeOH (90 mL) was added BrCN (2.77 g, 26.13 mmol, 1.92 mL, 3 eq) and the reaction mixture was stirred at 50° C. for 2 hrs under N₂ to afford a yellow suspension. LCMS showed the desired MS peak. The reaction mixture was concentrated under reduced pressure. The residue was triturated with EtOAc/i-PrOH (30/10 mL) to afford (E)-3-(4-(2-amino-5-carbamoyl-1H-benzo[d]imidazol-1-yl)but-2-en-1-yl)-2-(1-ethyl-3-methyl-1H-pyrazole-5-carboxamido)-3H-imidazo[4,5-b]pyridine-6-carboxamide (3.20 g, 5.06 mmol, 58.1% yield, 98.5% purity, HBr) yellow solid. LCMS: m/z 542.4 (M+1).

### Step 4

### (E)-3-(4-(5-carbamoyl-2-(1-ethyl-3-methyl-1H-pyrazole-5-carboxamido)-1H-benzo[d]imidazol-1-yl)but-2-en-1-yl)-2-(1-ethyl-3-methyl-1H-pyrazole-5-carboxamido)-3H-imidazo[4,5-b]pyridine-6-carboxamide

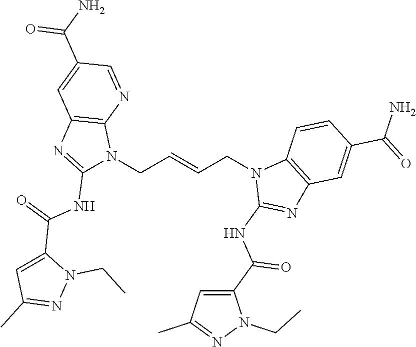 [SMILES: CCn1nc(C)cc1C(=O)Nc1nc2cc(C(N)=O)ccc2n1C/C=C/Cn1c(NC(=O)c2cc(C)nn2CC)nc2cc(C(N)=O)cnc21]

To a solution of (E)-3-(4-(2-amino-5-carbamoyl-1H-benzo[d]imidazol-1-yl)but-2-en-1-yl)-2-(1-ethyl-3-methyl-1H-pyrazole-5-carboxamido)-3H-imidazo[4,5-b]pyridine-6-carboxamide (1.00 g, 1.61 mmol, 1.00 eq, HBr), 1-ethyl-3-methyl-1H-pyrazole-5-carboxylic acid (297 mg, 1.93 mmol, 1.20 eq) and DIEA (1.04 g, 8.03 mmol, 1.40 mL, 5.00 eq) in DMF (15.0 mL) was added HATU (794 mg, 2.09 mmol, 1.30 eq) and the reaction mixture was stirred at 50° C. for 16 hrs, under N₂ to afford a yellow solution. LCMS showed the desired MS peak. The reaction mixture was filtered. The filter cake was washed with cold DMF (5 mL×2) and lyophilized. (E)-3-(4-(5-carbamoyl-2-(1-ethyl-3-methyl-1H-pyrazole-5-carboxamido)-1H-benzo[d]imidazol-1-yl)but-2-en-1-yl)-2-(1-ethyl-3-methyl-1H-pyrazole-5-carboxamido)-3H-imidazo[4,5-b]pyridine-6-carboxamide (1.40 g) as light yellow solid.

¹HNMR (400 MHz, DMSO-d₆): δ (ppm) 12.64-13.11 (m, 2H), 8.71 (s, 1H), 8.12 (s, 2H), 7.85-8.00 (m, 2H) 7.71 (d, J=8.13 Hz, 1H), 7.26-7.58 (m, 3H), 6.55 (s, 2H), 5.79-6.07 (m, 2H), 4.89-4.76 (m, 4H), 4.38-4.59 (m, 4H), 2.12 (s, 6H), 1.06-1.35 (m, 6H). LCMS: m/z 678.5 (M+1). HPLC: 93% purity.

### The Compound CF501 and its Synthesis

### (E)-3-(4-(5-carbamoyl-2-(1-ethyl-3-methyl-1H-pyrazole-5-carboxamido)-7-(3-morpholinopropoxy)-1H-benzo[d]imidazol-1-yl)but-2-en-1-yl)-2-(1-ethyl-3-methyl-1H-pyrazole-5-carboxamido)-3H-imidazo[4,5-b]pyridine-6-carboxamide

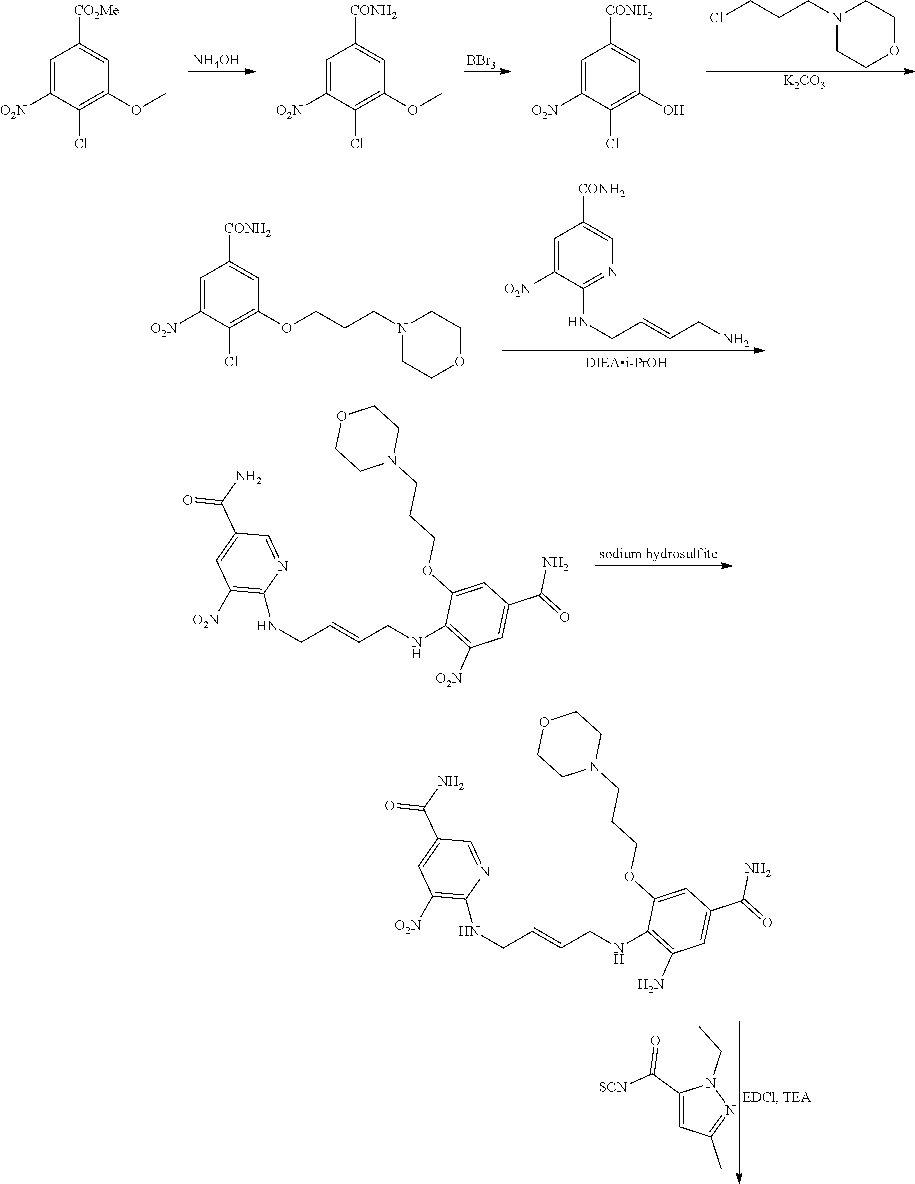

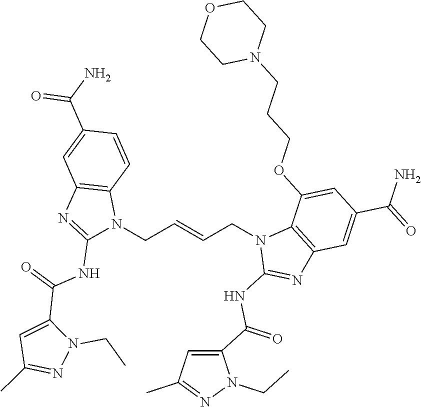 [SMILES: CCn1nc(C)cc1C(=O)Nc1nc2cc(C(N)=O)ccc2n1C/C=C/Cn1c(NC(=O)c2cc(C)nn2CC)nc2cc(C(N)=O)cc(OCCCN3CCOCC3)c21]

### Scheme of Synthesis

### Step 1: 4-Chloro-3-methoxy-5-nitrobenzamide

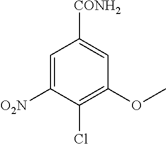

4-chloro-3-methoxy-5-nitrobenzoate (25 g, 10.1 mmol) was stirred in NH4OH (250 mL, 1.9 mmol) at room temperature for 24 hrs. The reaction temperature was then increased to 50° C. for 2 hrs. An additional 50 mL (˜3.7 eq) of NH4OH was added to the vessel. After an additional 2 hrs stirring at 50° C. the reaction mixture was cooled to room temperature. The solid was filtered and rinsed with cold water. The solid was dried under house vacuum and lyophilized to give 4-chloro-3-methoxy-5-nitrobenzamide (13 g, 68% yield) as a tan solid.

¹H NMR (400 MHz, DMSO-d₆): δ (ppm): 8.31 (br. s., 1H), 8.06 (d, J=1.8 Hz, 1H), 7.88 (d, 1=1.8 Hz, 1H), 7.81 (br. s., 1H), 4.02 (s, 3H). LCMS: m/z 230.9 (M+1).

### Step 2:4-Chloro-3-hydroxy-5-nitrobenzamide

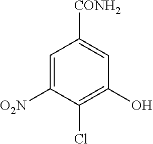 [SMILES: NC(=O)c1cc(O)c(Cl)c([N+](=O)[O-])c1]

4-Chloro-3-methoxy-5-nitrobenzamide (16 g, 69.3 mmol) was suspended in dry DCM (250 mL) and stirred at room temperature. To the reaction was added BBr3 (280 mL, IM in DCM) dropwisely. A slurry rapidly formed which was stirred overnight at room temperature under nitrogen. The reaction was poured into ice water (3 L) and stirred vigorously for 30 min. The resulting suspension was filtered and the solids dried to afford 4-chloro-3-hydroxy-5-nitrobenzamide (11.6 g, 77% yield). ¹H NMR (400 MHz, DMSO-d₆): δ (ppm): 11.53 (br. s., 1H), 8.17 (br. s., 1H), 7.92 (s, 1H), 7.72 (s, 1H), 7.66 (br. s., 1H).

LC-MS: [M+H]⁺=217. LCMS: m/z 217.0 (M+1).

### Step 3 4-Chloro-3-(3-morpholinopropoxy)-5-nitrobenzamide

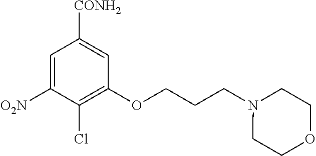 [SMILES: NC(=O)c1cc(OCCCN2CCOCC2)c(Cl)c([N+](=O)[O-])c1]

A mixture of 4-chloro-3-hydroxy-5-nitrobenzamide (11.6 g, 53.5 mmol), 4-(3-chloropropyl)morpholine (10.5 g, 64.2 mmol), K₂CO₃ (9.6 g, 69.6 mmol) in DMF (100 mL) was stirred at 70° C. overnight. Solvent was removed in vacuo to give a crude solid product that was purified by silica gel chromatography (MeOH:DCM=1:10) to give 4-chloro-3-(3-morpholinopropoxy)-5-nitrobenzamide (10.5 g, 57% yield) as a yellow solid. ¹H NMR (400 MHz, DMSO-d₆): δ (ppm): 8.30 (s, 1H), 8.05 (d, J=1.8 Hz, 1H), 7.88 (d, J=1.8 Hz, 1H), 7.80 (s, 1H), 4.28 (t, J=6.2 Hz, 2H), 3.57 (t, J=4.6 Hz, 4H), 2.41-2.47 (m, 2H), 2.37 (br. s., 4H), 1.97 (dd, J=13.94, 7.35 Hz, 2H); LCMS: m/z 344.1 (M+1).

### Step 4 (E)-6-((4-((4-carbamoyl-2-(3-morpholinopropoxy)-6-nitrophenyl)amino) but-2-en-1-yl)amino)-5-nitronicotinamide

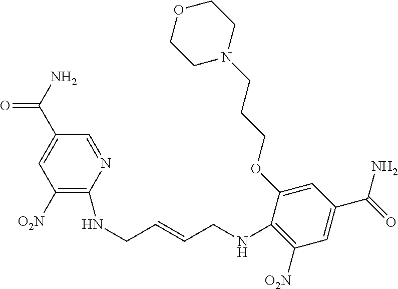 [SMILES: NC(=O)c1cnc(NC/C=C/CNc2c(OCCCN3CCOCC3)cc(C(N)=O)cc2[N+](=O)[O-])c([N+](=O)[O-])c1]

The solution of (E)-6-((4-aminobut-2-en-1-yl)amino)-5-nitronicotinamide (Intermediate 3, 220 mg, 0.87 mmol), 4-chloro-3-(3-morpholinopropoxy)-5-nitrobenzamide (200 mg, 0.58 mmol), i-PrOH (5 mL) and DIEA (1.12 g, 8.7 mmol) in a microwave vial was irradiated at 120° C. for 6 hrs. When cool, the resulting solid was isolated by filtration, rinsed with i-PrOH (2×1 mL) and dried to afford 6-((4-((4-carbamoyl-2-(3-morpholinopropoxy)-6-nitrophenyl)amino)but-2-en-1-yl)amino)-5-nitronicotinamide (113 mg, 35%) as a red solid.

### Step 5 (E)-5-amino-6-((4-((2-amino-4-carbamoyl-6-(3-morpholinopropoxy) phenyl)amino)but-2-en-1-yl)amino)nicotinamide

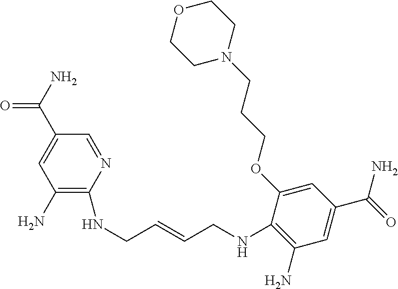 [SMILES: NC(=O)c1cnc(NC/C=C/CNc2c(N)cc(C(N)=O)cc2OCCCN2CCOCC2)c(N)c1]

To (E)-6-((4-((4-carbamoyl-2-(3-morpholinopropoxy)-6-nitrophenyl)amino)but-2-en-1-yl) amino)-5-nitronicotinamide (2.7 g, 4.8 mmol) in MeOH (40.0 mL) at room temperature was added sodium hydrosulfite (11.7 g, 67.2 mmol) in water (45 mL). After 15 min, solid sodium bicarbonate (24 g) was added. After 10 min., the reaction mixture was filtered and the solid was rinsed with MeOH (4×20 mL). The combined filtrates were concentrated onto Celite and purified by preparative HPLC to afford (E)-5-amino-6-((4-((2-amino-4-carbamoyl-6-(3-morpholinopropoxy)phenyl)amino)but-2-en-1-yl)amino)nicotinamide (1.81 g, 3.26 mmol, 49% yield) as a dark yellow solid. ¹H NMR (400 MHz, DMSO-d₆): δ (ppm): 7.93 (s, 1H), 7.60 (m, 1H), 7.10 (s, 1H), 6.96 (br s, 1H), 6.85 (m, 21), 6.77 (s, 1H), 6.14 (m, 1H), 5.73 (m, 2H), 4.81 (m, 2H), 4.66 (m 2H), 3.96 (m, 4H), 3.83 (m, 111), 3.54 (m, 611), 2.39 (t, J=7.2 Hz, 2H), 2.32 (br s, 4H), 1.84 (t, J=6.4 Hz, 2H). LCMS: m/z 499.3 (M+1).

### Step 6 (E)-3-(4-(5-carbamoyl-2-(1-ethyl-3-methyl-1H-pyrazole-5-carboxamido)-7-(3-morpholinopropoxy)-1H-benzo[d]imidazol-1-yl)but-2-en-1-yl)-2-(1-eth yl-3-methyl-1H-pyrazole-5-carboxamido)-3H-imidazo[4,5-b]pyridine-6-carboxamide

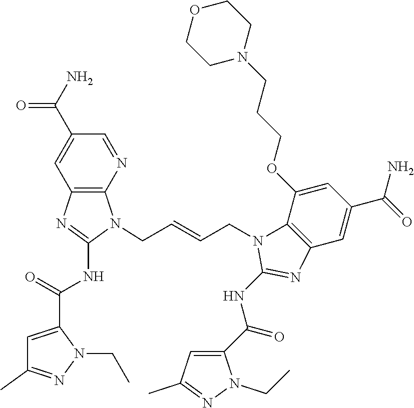 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN3CCOCC3)c21]

To

(E)-5-amino-6-((4-((2-amino-4-carbamoyl-6-(3-morpholinopropoxy)phenyl)amino)but-2-en-1-yl)amino)nicotinamide (812 mg, 1.62 mmol) in DMF (20 mL) at 0° C. was added 0.4 M 1-ethyl-3-methyl-1H-pyrazole-5-carbonyl isothiocyanate in dioxane (Intermediate 4, 6 mL, 3.89 mmol). After −10 min, another portion of 0.4 M 1-ethyl-3-methyl-1H-pyrazole-5-carbonyl isothiocyanate in dioxane (Intermediate 3, 2 ml, 0.48 mmol) was added, followed ˜15 min later by a final portion (2 ml, 0.48 mmol). After 35 min total reaction time, EDC (1.087 g, 5.67 mmol) was added followed by triethylamine (656 mg, 6.48 mmol). The mixture was warmed to room temperature and stirred overnight. The reaction was quenched with 3:1 water:saturated aqueous NH4Cl solution (10 mL) and extracted with 3:1 chloroform:ethanol (2×40 mL). The combined organic phases were washed with water (10 mL), dried over magnesium sulfate, and concentrated. The resulting residue was purified by preparative HPLC and the desired eluents were lyophilized to give (E)-3-(4-(5-carbamoyl-2-(1-ethyl-3-methyl-1H-pyrazole-5-carboxamido)-7-(3-morpholinopropoxy)-1H-benzo[d]imidazol-1-yl)but-2-en-1-yl)-2-(1-eth yl-3-methyl-1H-pyrazole-5-carboxamido)-3H-imidazo[4,5-b]pyridine-6-carboxamide as white solid (445 mg, yield 45%; HPLC purity: 97.7% (254 nm)).

¹H-NMR (400 MHz, DMSO-d₆): δ (ppm): 8.70 (s, 1H), 8.14 (s, 1H), 7.66 (s, 1H), 7.33 (s, 1H), 6.51 (d, J=12.0 Hz 2H), 5.95 (m, 1H), 5.74 (m, 1H), 4.94 (d, J=4.0 Hz, 2H), 4.79 (d, J=4.8 Hz, 2H), 4.52 (m, 4H), 4.13 (t, J=11.2 Hz 2H), 3.98 (m, 2H), 3.67 (s, 2H), 3.38 (t, J=12.4 Hz, 2H), 3.24 (m, 2H), 3.05 (m, 2H), 2.09 (s, 6H), 2.06 (m, 2H), 1.26 (t, J=14.0 Hz, 6H). LCMS: m/z 821.4 (M+1), 819.3 (M−1).

### The Compound CF508 and its Synthesis

### (E)-3-(4-(5-carbamoyl-2-(1-ethyl-3-methyl-1H-pyrazole-5-carboxamido)-7-(3-(piperazin-1-yl)propoxy)-1H-benzo[d]imidazol-1-yl)but-2-en-1-yl)-2-(1-ethyl-3-methyl-1H-pyrazole-5-carboxamido)-3H-imidazo[4,5-b]pyridine-6-carboxamide

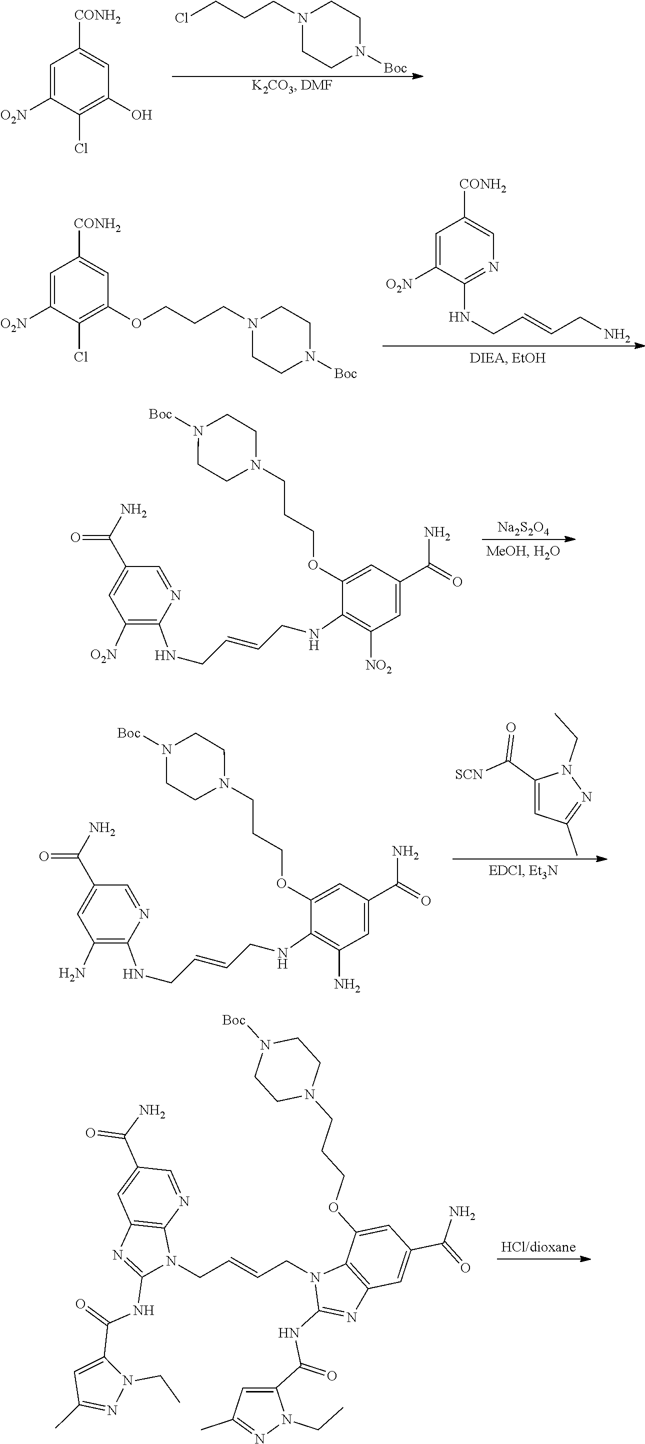

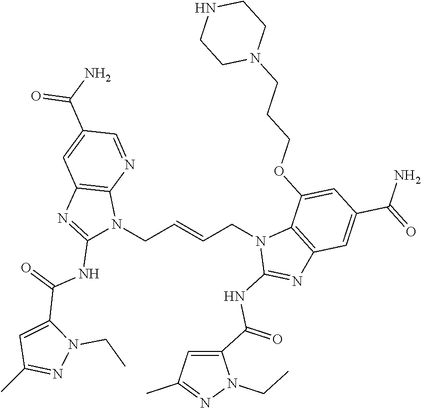 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN3CCNCC3)c21]

### Scheme of Synthesis

### Step 1

### tert-butyl4-(3-(5-carbamoyl-2-chloro-3-nitrophenoxy)propyl)piperazine-1l-carboxylate

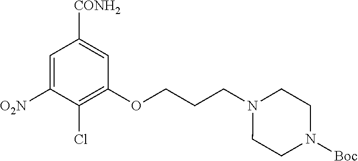 [SMILES: CC(C)(C)OC(=O)N1CCN(CCCOc2cc(C(N)=O)cc([N+](=O)[O-])c2Cl)CC1]

A mixture of 4-chloro-3-hydroxy-5-nitrobenzamide (prepared as example 3, 3.20 g, 14.8 mmol, 1.00 eq), tert-butyl 4-(3-chloropropyl)piperazine-1-carboxylate (4.66 g, 17.7 mmol, 1.20 eq) and K₂CO₃ (2.65 g, 19.2 mmol, 1.30 eq) in DMF (32 mL), the mixture was stirred at 70° C. for 16 hrs under N₂ atmosphere to give a yellow solution. LC-MS showed the starting material was consumed completely and one main peak with desired MS was detected. The mixture was concentrated under reduced pressure to give a crude product. The crude product was triturated with H₂O (100 mL) at 25° C. for 16 hrs. to provide tert-butyl 4-(3-(5-carbamoyl-2-chloro-3-nitrophenoxy)propyl)piperazine-1-carboxylate (6.00 g, 13.6 mmol, 91.7% yield) as a yellow solid.

HNMR (400 MHz, DMSO-d6): δ 8.34 (br s, 1H), 8.05 (s, 1H), 7.89 (s, 1H), 7.80 (br s, 1H), 4.28 (br t, J=5.4 Hz, 2H), 3.37 (br s, 12H), 2.17-2.38 (m, 5H), 1.86-2.08 (m, 2H), 1.39 (s, 121), LCMS: m/z (ES+) [M+H]+=443.5.

### Step 2

### tert-butyl(E)-4-(3-(5-carbamoyl-2-((4-((5-carbamoyl-3-nitropyridin-2-yl)amino)but-2-en-1-yl)amino)-3-nitrophenoxy)propyl)piperazine-1-carboxylate

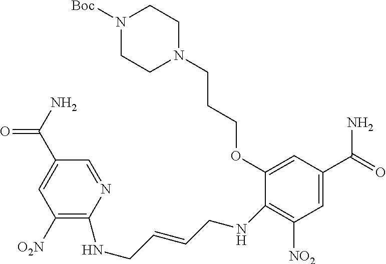 [SMILES: CC(C)(C)OC(=O)N1CCN(CCCOc2cc(C(N)=O)cc([N+](=O)[O-])c2NC/C=C/CNc2ncc(C(N)=O)cc2[N+](=O)[O-])CC1]

A solution of (E)-6-((4-aminobut-2-en-1-yl)amino)-5-nitronicotinamide (Intermediate 3, 9.74 g, 33.9 mmol, 1.50 eq, HCJ), tert-butyl 4-(3-(5-carbamoyl-2-chloro-3-nitrophenoxy)propyl)piperazine-1-carboxylate (10.0 g, 22.6 mmol, 1.00 eq), DIEA (11.7 g, 90.3 mmol, 15.7 mL, 4.00 eq) and NaHCO₃ (7.59 g, 90.3 mmol, 3.51 mL, 4.00 eq) in EtOH (110 mL) was stirred at 110° C. for 16 hrs under N₂ of sealed tube to afford a yellow suspension. LC-MS showed the starting material was consumed completely and one main peak with desired MS was detected. The mixture was concentrated to give crude product. The residue was purified by flash silica gel chromatography (ISCO®; 120 g SepaFlash® Silica Flash Column, Eluent of 5-10% Methanol/Dichloromethane @ 100 mL/min_Dichloromethane/Methanol=10:1, R_{f} of product=0.50, UV 254 nm) to produce tert-butyl (E)-4-(3-(5-carbamoyl-2-((4-((5-carbamoyl-3-nitropyridin-2-yl)amino)but-2-en-1-yl)amino)-3-nitrophenoxy)propyl)piperazine-1-carboxylate (5.50 g, 7.51 mmol, 33.2% yield, 89.8% purity) as red brown solid.

¹HNMR (400 MHz, DMSO-d6): δ 8.73-8.99 (m, 3H), 7.99-8.23 (m, 3H), 7.71-7.85 (m, 1H), 7.41-7.60 (m, 2H), 7.34 (br s, 1H), 5.56-5.87 (m, 211), 4.38 (br s, 1H), 4.08-4.26 (m, 4H), 4.05 (br t, J=6.1 Hz, 2H), 3.53-3.69 (m, 1H), 3.03-3.49 (m, 15f), 2.19-2.46 (m, 6H), 1.90 (br s, 2H), 1.39 (s, 9H), 1.17-1.31 (m, 5H), 1.06 (t, J=7.0 Hz, 3H); LCMS: m/z (ES+) [M+H]⁺=658.2.

### Step 3

### tert-butyl(E)-4-(3-(3-amino-2-((4-((3-amino-5-carbamoylpyridin-2-yl)amino)but-2-en-1-yl)amino)-5-carbamoylphenoxy)propyl)piperazine-1-carboxylate

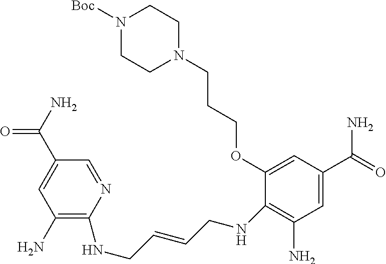 [SMILES: CC(C)(C)OC(=O)N1CCN(CCCOc2cc(C(N)=O)cc(N)c2NC/C=C/CNc2ncc(C(N)=O)cc2N)CC1]

To a solution of tert-butyl (E)-4-(3-(5-carbamoyl-2-((4-((5-carbamoyl-3-nitropyridin-2-yl)amino)but-2-en-1-yl)amino)-3-nitrophenoxy)propyl)piperazine-1-carboxylate (5.50 g, 8.36 mmol, 1.00 eq) in MeOH (110 mL) and H₂O (55.0 mL) was added NaHCO₃ (42.0 g, 500 mmol, 19.5 mL, 59.8 eq) followed by disodium; BLAH (20.4 g, 117 mmol, 25.5 mL, 14.0 eq) at 0° C. and then the reaction mixture was stirred at 20° C. for 2 hrs. A light yellow suspension was obtained. LCMS showed Reactant 1 was consumed completely and one main peak with desired MS was detected. The reaction mixture was filtered and the filter cake was washed with MeOH (100 mL*2). The filtrate was concentrated. The crude product tert-butyl (E)-4-(3-(3-amino-2-((4-((3-amino-5-carbamoylpyridin-2-yl)amino)but-2-en-1-yl)amino)-5-carbamoylphenoxy)propyl)piperazine-1-carboxylate (5.00 g, 8.37 mmol, 100% yield) was used directly in the next step without purification. LCMS: m/z (ES+) [M+H]⁺=598.2;

### Step 4 tert-butyl(E)-4-(3-((5-carbamoyl-1-(4-(6-carbamoyl-2-(1-ethyl-3-methyl-1H-pyrazole-5-carboxamido)-3H-imidazo[4,5-b]pyridin-3-yl)but-2-en-1-yl)-2-(1-ethyl-3-methyl-1H-pyrazole-5-carboxamido)-1H-benzo[d]imidazol-7-yl)oxy)propyl)piperazine-1-carboxylate

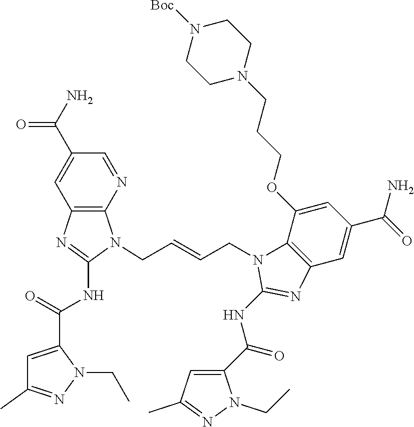 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN3CCN(C(=O)OC(C)(C)C)CC3)c21]

To a solution of tert-butyl (E)-4-(3-(3-amino-2-((4-((3-amino-5-carbamoylpyridin-2-yl)amino)but-2-en-1-yl)amino)-5-carbamoylphenoxy)propyl)piperazine-1-carboxylate (5.00 g, 8.37 mmol, 1.00 eq) in DMF (100 mL) was added a solution of 1-ethyl-3-methyl-1H-pyrazole-5-carbonyl isothiocyanate (Intermediate 4, 4.41 g, 22.6 mmol, 2.70 eq) in dioxane (20 mL) of 0 min (3 mL), 10 min (3 mL), 15 min (3 mL) at 0° C., respectively. The reaction mixture was stirred at 0° C. for 30 min, then EDCI (5.61 g, 29.3 mmol, 3.50 eq) and Et₃N (6.77 g, 66.9 mmol, 9.31 mL, 8.00 eq) was added at 0° C., then heated to 25° C. and stirred for 2 hrs to afford a yellow solution. LC-MS showed the starting material was consumed completely and desired compound was detected. The reaction mixture was quenched with a saturated aqueous NH₄Cl solution (50 mL). The solution was filtered and the filtrate was concentrated. The crude product was purified by prep-HPLC (column: Phenomenex Gemini YMC Triart C18 250*50 mm*7 um; mobile phase: [water (10 mM NH4OAc)-ACN, 30-49, 30 min) to provide tert-butyl (E)-4-(3-((5-carbamoyl-1-(4-(6-carbamoyl-2-(1-ethyl-3-methyl-1H-pyrazole-5-carboxamido)-3H-imidazo[4,5-b]pyridin-3-yl)but-2-en-1-yl)-2-(1-ethyl-3-methyl-1H-pyrazole-5-carboxamido)-1H-benzo[d]imidazol-7-yl)oxy)propyl)piperazine-1-carboxylate (2.50 g, 2.42 mmol, 28.9% yield, 89.0% purity) as white solid. LCMS: m/z (ES+) [M+H]⁺=920.5.

### Step 5

### (E)-3-(4-(5-carbamoyl-2-(1-ethyl-3-methyl-1H-pyrazole-5-carboxamido)-7-(3-(piperazin-1-yl)propoxy)-1H-benzo[d]imidazol-1-yl)but-2-en-1-yl)-2-(1-ethyl-3-methyl-1H-pyrazole-5-carboxamido)-3H-imidazo[4,5-b]pyridine-6-carboxamide

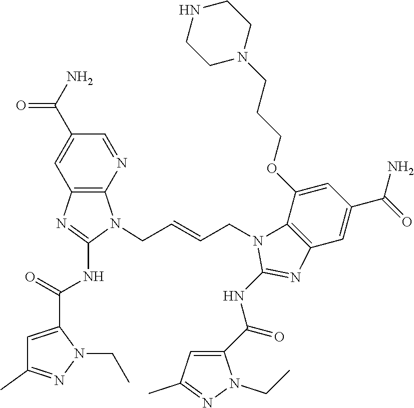 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN3CCNCC3)c21]

To a solution of tert-butyl (E)-4-(3-((5-carbamoyl-1-(4-(6-carbamoyl-2-(1-ethyl-3-methyl-1H-pyrazole-5-carboxamido)-3H-imidazo[4,5-b]pyridin-3-yl)but-2-en-1-yl)-2-(1-ethyl-3-methyl-1H-pyrazole-5-carboxamido)-1H-benzo[d]imidazol-7-yl)oxy)propyl)piperazine-1-carboxylate (900 mg, 978 μmol, 1.00 eq) in MeOH (15 mL) and dioxane (15 mL) was added HCl/dioxane (4 M, 30 mL, 123 eq) and the mixture was stirred at 25° C. for 16 hrs under N₂ to afford a yellow solution. LC-MS showed the starting material was consumed completely and desired compound was detected. The mixture was concentrated under reduced pressure to give a residue. The crude product was purified by prep-HPLC (column: Phenomenex Gemini YMC Triart C18 250×50 mm×7 μm; mobile phase: [water (0.05% HCl)-ACN, 20-80 30 min) to provide (E)-3-(4-(5-carbamoyl-2-(1-ethyl-3-methyl-1H-pyrazole-5-carboxamido)-7-(3-(piperazin-1-yl)propoxy)-1H-benzo[d]imidazol-1-yl)but-2-en-1-yl)-2-(1-ethyl-3-methyl-1H-pyrazole-5-carboxamido)-3H-imidazo[4,5-b]pyridine-6-carboxamide (150 mg, 0.176 mmol, 17.9% yield, 96.0% purity) as white solid.

¹HNMR (400 MHz, DMSO-d6): δ 11.89-12.32 (m, 1H), 9.57-10.02 (m, 2H), 8.78 (d, J=1.8 Hz, 1H), 8.09-8.33 (m, 2H), 7.99 (br s, 1H), 7.66 (d, J=1.0 Hz, 1H), 7.56 (br s, 1H), 7.26-7.49 (m, 2H), 6.53 (s, 1H), 6.48 (s, 1H), 6.00 (dt, J=15.5, 5.1 Hz, 1H), 5.73 (dt, J=15.6, 5.6 Hz, 1H), 4.98 (br d, J=3.6 Hz, 2H), 4.81 (br d, J=5.3 Hz, 2H), 4.39-4.58 (m, 4H), 3.21-3.80 (m, 9H), 2.13-2.30 (m, 3H), 2.09 (d, J=4.9 Hz, 6H), 1.25 ppm (td, J=7.1, 3.9 Hz, 6H); LCMS: m/z (ES+) [M+H]⁺=920.4.

### The Compound CF510 and its Synthesis

### (E)-3-(4-(5-carbamoyl-2-(1-ethyl-3-methyl-1H-pyrazole-5-carboxamido)-7-(2-morpholinoethoxy)-1H-benzo[d]imidazol-1-yl)but-2-en-1-yl)-2-(1-ethyl-3-methyl-1H-pyrazole-5-carboxamido)-3H-imidazo[4,5-b]pyridine-6-carboxamide

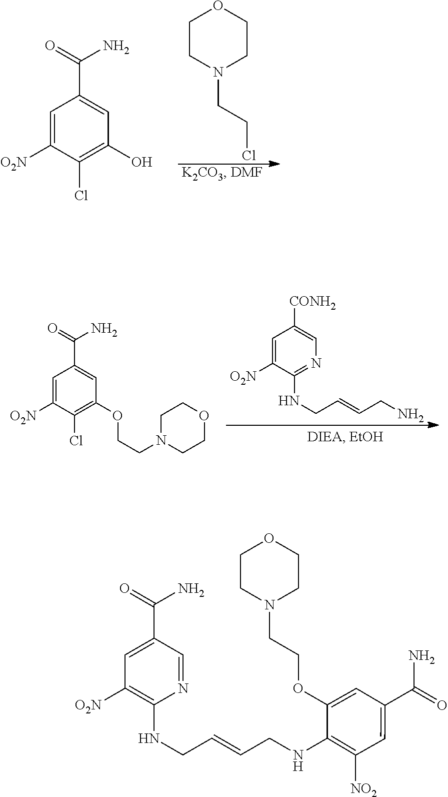

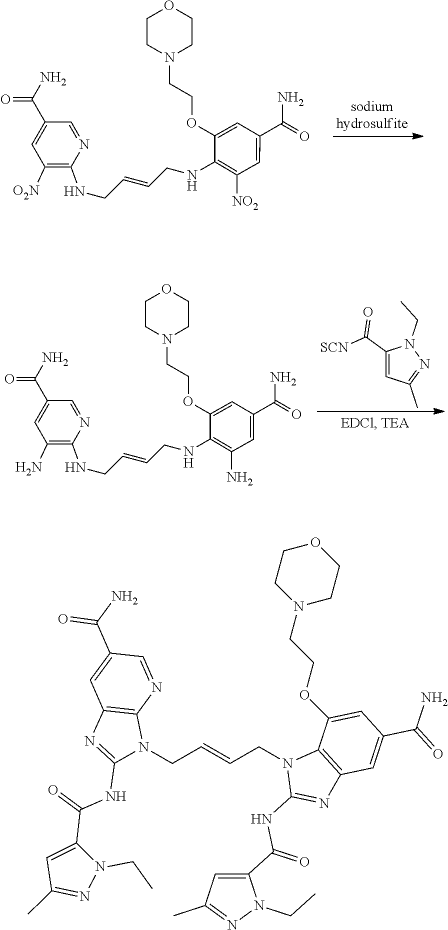

### Scheme of Synthesis

### Step 1 4-chloro-3-(2-morpholinoethoxy)-5-nitrobenzamide

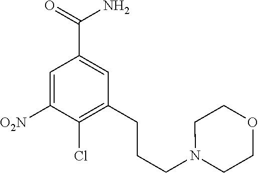 [SMILES: NC(=O)c1cc(CCCN2CCOCC2)c(Cl)c([N+](=O)[O-])c1]

A mixture of 4-chloro-3-hydroxy-5-nitrobenzamide (prepared as example 3, 1.00 g, 4.62 mmol, 1.00 eq), 4-(2-chloroethyl)morpholine (1.03 g, 5.54 mmol, 1.20 eq, HCl) and K₂CO₃ (1.28 g, 9.23 mmol, 2.00 eq) in DMF (15.0 mL), the mixture was stirred at 70° C. for 16 hrs under N₂ atmosphere to give a yellow solution. LC-MS showed the starting material was consumed completely and one main peak with desired MS was detected. The mixture was concentrated under reduced pressure to create a crude product. The crude product was triturated with H₂O (50.0 mL) at 25° C. for 16 hrs. 4-chloro-3-(2-morpholinoethoxy)-5-nitrobenzamide (2.60 g, 7.65 mmol, 82.8% yield, 97.0% purity) was obtained as a yellow solid. LCMS: m/z (ES+) [M+H]⁺=330.1.

### Step 2 (E)-6-((4-((4-carbamoyl-2-(2-morpholinoethoxy)-6-nitrophenyl)amino)but-2-en-1-yl) amino)-5-nitronicotinamide

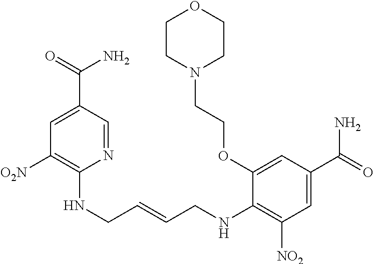 [SMILES: NC(=O)c1cnc(NC/C=C/CNc2c(OCCN3CCOCC3)cc(C(N)=O)cc2[N+](=O)[O-])c([N+](=O)[O-])c1]

A solution of 4-chloro-3-(2-morpholinoethoxy)-5-nitrobenzamide (1.05 g, 3.64 mmol, 1.20 eq, HCl), NaHCO₃ (1.02 g, 12.1 mmol, 472 uL, 4.00 eq) and 4-chloro-3-(2-morpholinoethoxy)-5-nitro-benzamide (Intermediate 3, 1.00 g, 3.03 mmol, 1.00 eq), DIEA (1.57 g, 12.1 mmol, 2.11 mL, 4.00 eq) in EtOH (10.0 mL) was stirred at 110° C. for 16 hrs under N₂ to afford a yellow suspension. LCMS showed the starting material was consumed completely. The residue was purified by flash silica gel chromatography (ISCO®; 330 g SepaFlash® Silica Flash Column, Eluent of 2-5% Methanol/Dichloromethane @ 100 mL/min_Dichloromethane/Methanol=10:1, Rₜ of product=0.27, UV 254 nm). (E)-6-((4-((4-carbamoyl-2-(2-morpholinoethoxy)-6-nitrophenyl)amino)but-2-en-1-yl)amino)-5-nitronicotinamide (1.34 g, 2.12 mmol, 69.8% yield) was obtained as a red brown solid.

¹HNMR (400 MHz, DMSO-d6): δ 0.77-0.85 (m, 6H) 1.11 (t, J=7.13 Hz, 4H) 3.49 (br d, J=6.13 Hz, 2H) 3.75-4.25 (m, 12H) 5.56-5.74 (m, 2H) 6.82-6.96 (m, 1H) 7.78 (br t, J=6.00 Hz, 1H) 7.93-8.15 (m, 3H) 8.72-8.91 (m, 3H); LCMS: m/z (ES+) [M+H]⁺=545.2.

### Step 3 (E)-5-amino-6-((4-((2-amino-4-carbamoyl-6-(2-morpholinoethoxy)phenyl)amino)but-2-en-1-yl)amino)nicotinamide

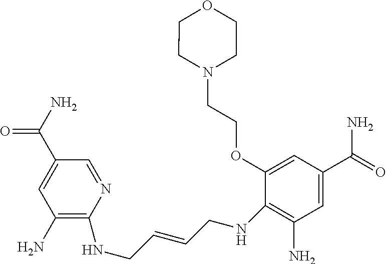 [SMILES: NC(=O)c1cnc(NC/C=C/CNc2c(N)cc(C(N)=O)cc2OCCN2CCOCC2)c(N)c1]

To a solution of (E)-6-((4-((4-carbamoyl-2-(2-morpholinoethoxy)-6-nitrophenyl)amino)but-2-en-1-yl)amino)-5-nitronicotinamide (1.00 g, 1.84 mmol, 1.00 eq) in MeOH (20 mL) and H₂O (15.0 mL) was added NaHCO₃ (9.00 g, 107 mmol, 4.17 mL, 58.3 eq) followed by disodium; BLAH (4.48 g, 25.7 mmol, 5.60 mL, 14.0 eq) at 0° C. and then the reaction mixture was stirred at 20° C. for 2 hrs. A light yellow suspension was obtained. LCMS showed the starting material was consumed completely and one main peak with desired MS was detected. The reaction mixture was filtered and the filter cake was washed with MeOH (50.0 mL). The filtrate was concentrated. The crude product was used directly in the next step without purification. (E)-5-amino-6-((4-((2-amino-4-carbamoyl-6-(2-morpholinoethoxy)phenyl)amino)but-2-en-1-yl)amino)nicotinamide (889 mg, 1.84 mmol, 100% yield) was obtained as a yellow solid. LCMS: m/z (ES+) [M+H]⁺=484.3.

### Step 4 (E)-3-(4-(5-carbamoyl-2-(1-ethyl-3-methyl-1H-pyrazole-5-carboxamido)-7-(2-morpholinoethoxy)-1H-benzo[d]imidazol-1-yl)but-2-en-1-yl)-2-(1-ethyl-3-methyl-1H-pyrazole-5-carboxamido)-3H-imidazo[4,5-b]pyridine-6-carboxamide

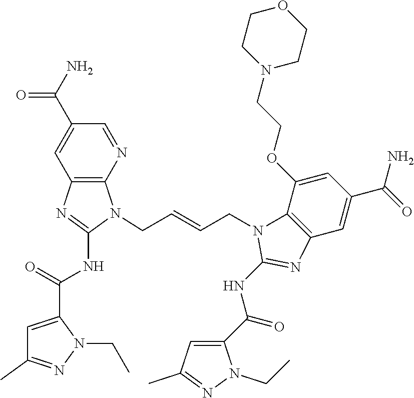 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCN3CCOCC3)c21]

To a solution of the (E)-5-amino-6-((4-((2-amino-4-carbamoyl-6-(2-morpholinoethoxy)phenyl)amino)but-2-en-1-yl)amino)nicotinamide (889 mg, 1.84 mmol, 1.00 eq), in DMF (23.0 mL) was added a solution of 2-ethyl-5-methyl-pyrazole-3-carbonyl isothiocyanate (Intermediate 4, 968 mg, 4.96 mmol, 2.70 eq) in dioxane (5.30 mL) of 0 min (3.00 mL), 10 min (3.00 mL), 15 min (3.00 mL) at 0° C., respectively. The reaction mixture was stirred at 0° C. for 35 mins, then EDCI (1.23 g, 6.43 mmol, 3.50 eq) and Et₃N (1.49 g, 14.7 mmol, 2.04 mL, 8.00 eq) were added at 0° C., then heated to 25° C. and stirred for 16 hrs to afford a yellow solution. LCMS showed Reactant 1 was consumed completely and desired compound was detected. The reaction mixture was quenched with a saturated aqueous NH₄Cl solution (50.0 mL). The solution was filtered and the filtrate was concentrated. The crude product was purified by prep-HPLC (column: Phenomenex Gemini C18 250×50 mm×7 um; mobile phase: [water (10 mM HCl)-ACN]; B %: 10%-40%, 20 min) to provide (E)-3-(4-(5-carbamoyl-2-(1-ethyl-3-methyl-1H-pyrazole-5-carboxamido)-7-(2-morpholino ethoxy)-1H-benzo[d]imidazol-1-yl)but-2-en-1-yl)-2-(1-ethyl-3-methyl-1H-pyrazole-5-carboxamido)-3H-imidazo[4,5-b]pyridine-6-carboxamide (52.0 mg, 0.06 mmol, 3.27% yield) as a white solid.

¹HNMR (400 MHz, DMSO-d6): δ 1.19-1.36 (m, 8H) 2.03-2.25 (m, 9H) 2.26-2.35 (m, 5H) 2.68 (br s, 1H) 3.45-3.53 (m, 3H) 4.11 (br t, J=5.63 Hz, 2H) 4.45-4.61 (m, 4H) 4.79 (br s, 2H) 4.97 (br s, 2H) 5.87-5.99 (m, 2H) 6.54 (s, 2H) 7.34 (s, 2H) 7.53 (br s, 1H) 7.64 (s, 1H) 7.93 (br s, 1H) 8.12-8.19 (m, 2H) 8.71 (s, 1H); LCMS: m/z (ES+) [M+H]+=807.3.

The compound CF512 and its synthesis

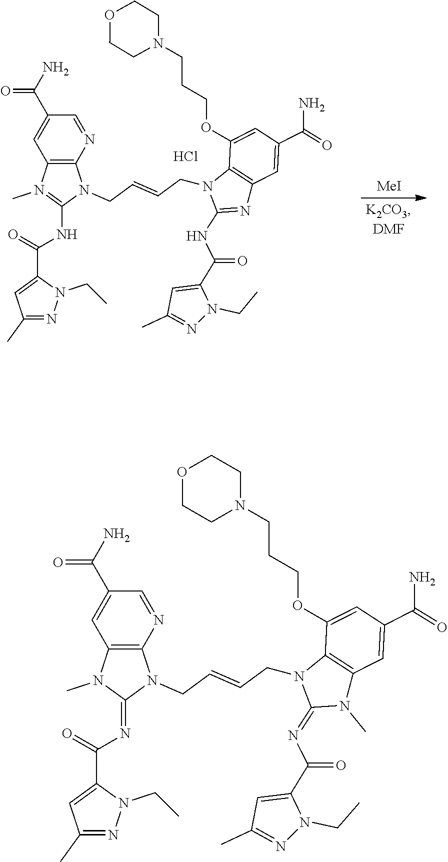

### Scheme of Synthesis

To a solution of (E)-3-(4-(5-carbamoyl-2-(1-ethyl-3-methyl-1H-pyrazole-5-carboxamido)-7-(3-morpholinopropoxy)-1H-benzo[d]imidazol-1-yl)but-2-en-1-yl)-2-(1-eth yl-3-methyl-1H-pyrazole-5-carboxamido)-3H-imidazo[4,5-b]pyridine-6-carboxamide (Example 4, 400 mg, 487 umol, 1.00 eq) and K₂CO₃ (148 mg, 1.07 mmol, 2.20 eq) in DMF (8 mL) was added a solution of Mel (3.05 g, 21.5 mmol, 1.34 mL, 44.1 eq) in DMF (0.8 mL) at 25° C. and the reaction stirred at 25° C. for 16 hrs to give a yellow solution. The mixture was concentrated under reduced pressure to give a residue. The crude product was purified by prep-HPLC (column: Phenomenex Gemini C18 150*25 mm*10 um; mobile phase: [water (0.05% NH₃H₂O+10 mM NH₄HCO₃)-ACN]; B %: 10%-40%, 20 min) to produce (E)-3-((E)-4-((E)-5-carbamoyl-2-((1-ethyl-3-methyl-1H-pyrazole-5-carbonyl)imino)-3-methyl-7-(3-morpholinopropoxy)-2,3-dihydro-1H-benzo[d]imidazol-1-yl)but-2-en-1-yl)-2-((1-ethyl-3-methyl-1H-pyrazole-5-carbonyl)imino)-1-methyl-2,3-dihydro-1H-imidazo[4,5-b]pyridine-6-carboxamide (120 mg, 0.138 mmol, 28.4% yield, 97.9% h purity) as white solid.

¹HNMR (400 MHz, DMSO-d6): δ 8.74 (d, J=1.6 Hz, 1H), 8.35 (d, J=1.6 Hz, 1H), 8.14 (br s, 1H), 8.04 (br s, 1H), 7.71 (s, 1H), 7.61 (br s, 1H), 7.36-7.49 (m, 2H), 6.40 (s, 1H), 6.35 (s, 1H), 5.75-5.88 (m, 1H), 5.60-5.75 (m, 1H), 4.80 (br dd, J=16.4, 5.3 Hz, 4H), 4.30-4.54 (m, 4H), 4.07 (br t, J=6.3 Hz, 2H), 3.40-3.58 (m, 10H), 2.18-2.33 (m, 6H), 2.10 (d, J=13.3 Hz, 6H), 1.73 (q, J=6.5 Hz, 2H), 1.21 (dt, J=8.9, 7.2 Hz, 6H); LCMS: m/z (ES+) [M+H]+=849.6.

### Example 1: Detection of the Innate Immune Response Activated by STING Agonists in Mice

1. Materials:

8-week-old SPF C57 mice were purchased from Beijing Vital River Laboratory Animal Technology Co. Ltd. The STING agonist, 3′-3′ cGAMP (abbreviated as cGAMP below) was purchased from Invivogen. The Trizol was purchased from Takara Bio Inc. The reverse transcription kit (PrimeScript™ RT reagent Kit with gDNA Eraser (Perfect Real Time)) was purchased from Takara BioInc. The fluorescence quantitative PCR kit (TB Green® Premix DimerEraser™ (Perfect Real lime)) was purchased from Takara BioInc.

2.1 The Detection of the Innate Immune Response in Mice Activated by Intramuscular Injection of STING Agonists

2.1.1 Experimental Methods
(1) 12 SPF C57 mice were divided into three groups of 4 mice each randomly. The mice were injected with 20 μg of the STING agonists CF501, cGAMP, or an equal volume of PBS by intramuscular injection, respectively.
(2) After 6 hrs, the mice were euthanized, the draining lymph nodes of the mice were isolated, and the total RNA of the lymph nodes was extracted by the Trizol method.
(3) The reverse transcription of the extracted RNA into cDNA was performed using the reverse transcription kit.
(4) IFNb, CXCL-10, CXCL-9, CCL-2, TNFα, IL-1β, and IL-6 in lymph nodes of the mice were detected using the fluorescent quantitative PCR kit.

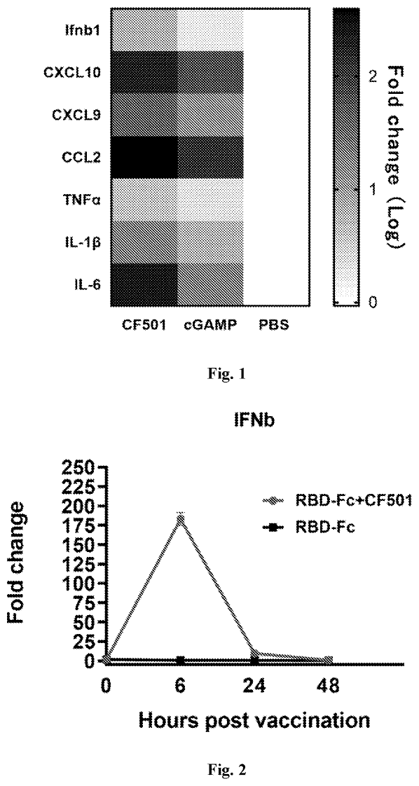

As shown in FIG. 1, the STING agonist CF501 and cGAMP can activate a large production of the cytokines at 6 hrs after the intramuscular injection. However, CF501 can activate the production of these cytokines which are detected more potently than cGAMP.

2.2 Monitoring the Innate Immune Responses of Mice Activated by the Intramuscular Injection with CF501 and the SARS-CoV-2 RBD-Fc Protein at Different Times.

2.2.1 Experimental Methods
(1) 21 SPF C57 mice were divided randomly. Three mice in Group 1 were directly euthanized, and the draining lymph nodes were taken as controls before administration. 9 mice in Group 2 were injected intramuscularly with 5 μg of the SARS-CoV-2 RBD-Fc protein (Kactus Biosystems Co. Ltd, catalog number: COV-VM5BD; also abbreviated as RBD-Fc or RBD-Fc protein below), and three mice were euthanized separately at 6 hrs, 24 hrs and 48 hrs after the injection to collect draining lymph nodes. In Group 3, 9 mice were injected intramuscularly with 5 μg of the RBD-Fc protein and 20 μg of CF501, and 3 mice were euthanized separately at 6 hrs, 24 hrs and 48 hrs to collect the draining lymph nodes.
(2) The Trizol method was used to extract RNA from the collected lymph nodes.
(3) The reverse transcription kit and fluorescence quantitative kit were used to detect the dynamic change level of each cytokine.

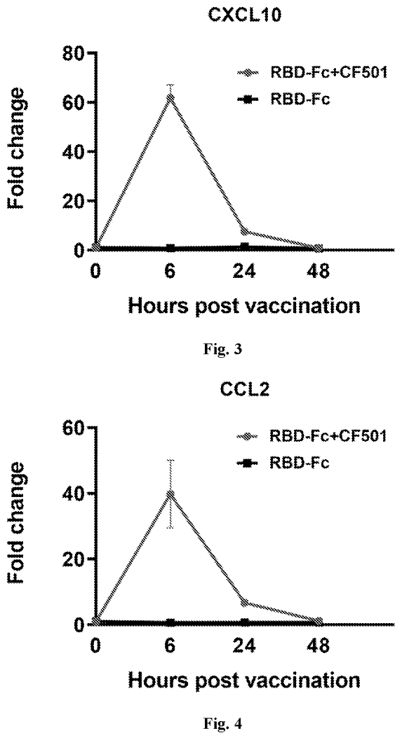

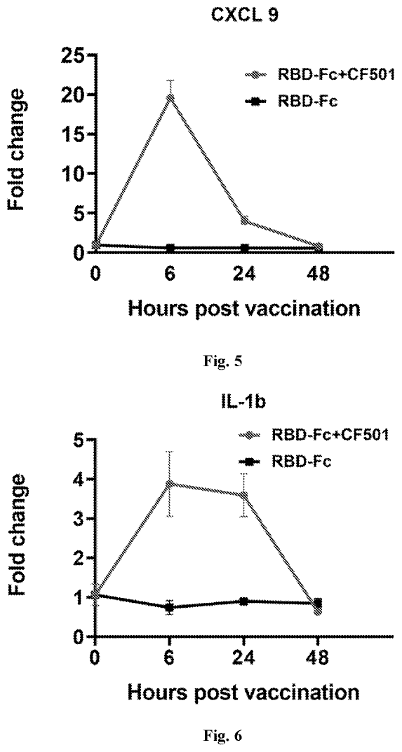

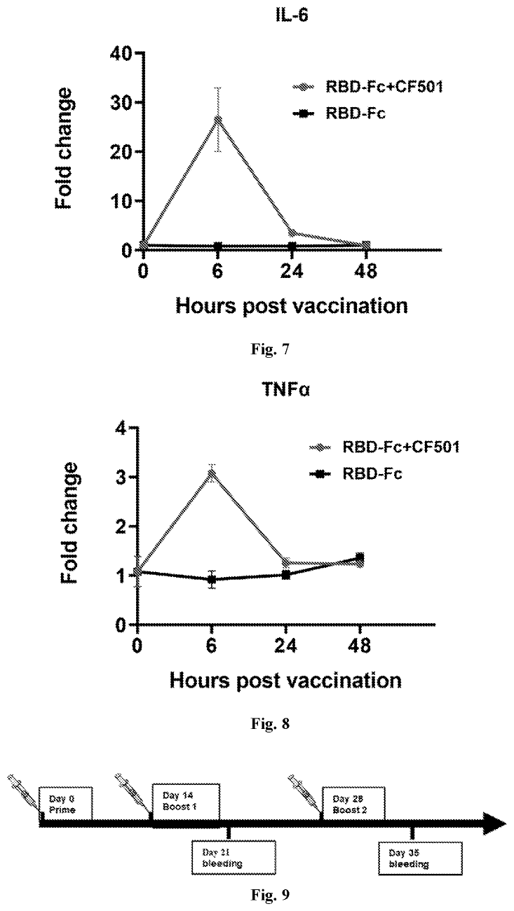

As shown in FIGS. 2-8, after the intramuscular injection of CF501 and the SARS-CoV-2 RBD-Fc protein into the mice, the innate immune response is strongly activated at 6 hrs, while the RBD-Fc alone cannot effectively activate the innate immune response. Although the mice vaccinated with the SARS-CoV-2 RBD-Fc protein mixed with CF501 can have strongly increased levels of various cytokines at 6 hrs, the levels of the cytokines tend to be normal at 48 hrs. This indicates that CF501 only transiently activates the innate immune response of the mice, and does not cause inflammation in the mice. The transient activation of the innate immune response is strong, but the compound does not continuously cause the up-regulation of the cytokines, indicating that it has a superior effect to activate the innate immune response and is safe.

### Example 2: Detection of Antibody Immune Response in Mice Vaccinated with STING Agonists as Adjuvant and the SARS-CoV-2 RBD-Fc Protein

1. Experimental Materials

6-week-old SPF Balb/c mice were purchased from Beijing Vital River Laboratory Animal Technology Co. Ltd. Aluminum adjuvant was purchased from Thermo Scientific. cGAMP was purchased from Invivogen.

2. Experimental Methods.

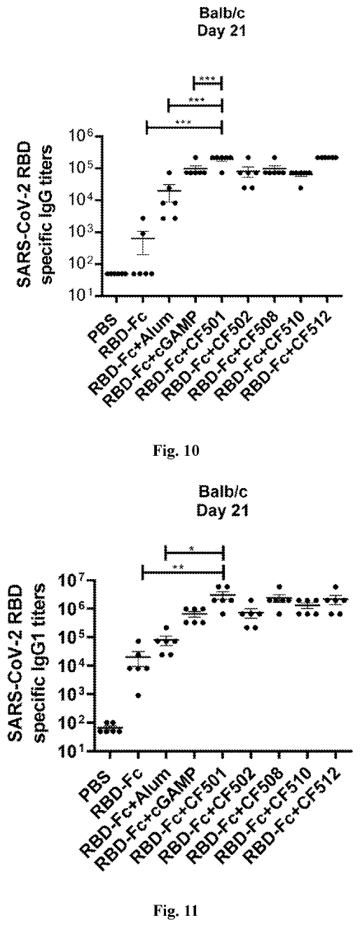

The procedure for the vaccination of the mice is shown in FIG. 9.

The steps are as follows.
(1) 54 mice were divided into 9 groups of 6 mice each randomly.
(2) Group 1 of the mice was injected with 5 μg of the RBD-Fc protein intramuscularly. Group 2 of the mice were injected with 5 μg of the RBD-Fc protein and an equal volume of aluminum adjuvant intramuscularly. Group 3 of the mice were injected with 5 μg of the RBD-Fc protein and 20 μg of CF501 (RBD-Fc+CF501) intramuscularly. Group 4 of the mice were injected with 5 μg of the RBD-Fc protein and 20 μg of the STING agonist CF502. Group 5 of the mice were injected with 5 μg of the RBD-Fc protein and 20 Mg of the STING agonist CF508. Group 6 of the mice were injected with 5 Mg of the RBD-Fc protein and 20 μg of the STING agonist CF510. Group 7 of the mice were injected with 5 Mg of the RBD-Fc protein and 20 g of the STING agonist CF512. Group 8 of the mice were injected with 5 μg of the RBD-Fc protein and 20 μg of cGAMP. Group 9 of the mice were injected with an equal volume of PBS.
(3) The second booster immunization was performed at day 14 after the first immunization. Blood was taken from the mice at day 21 after the first immunization to obtain the sera. The third booster immunization was performed at Day 28 after the first immunization. Blood was taken from the mice at day 35 after the first immunization to obtain the sera.
(4) The ELISA method was used to detect the titer of the RBD specific antibodies in the sera. In particular, 1 μg/ml of the RBD-His protein (Kactus Biosystems Co. Ltd, catalog number: COV-VM4BD) was coated onto an ELISA plate. After the plate was blocked with 5% skimmed milk powder, the sera were diluted serially in 3 or 4 folds, added to the ELISA plate, and incubated at 37° C. for 30 min. After the plate was washed 5 times with PBST, the HRP-labeled rabbit anti-mouse IgG, the HRP-labeled rabbit anti-mouse IgG1 and the HRP-labeled rabbit anti-mouse IgG2a antibodies were added respectively, and the plate was incubated at 37° C. for 30 min. After the plate was washed with PBST, the substrate TMB was added for color development for 15 mins and then H₂SO₄ was added to stop the reaction. A microplate reader was used to detect the OD450. The highest dilution at which the OD450 is greater than the OD450 of the blank control group (no serum but PBS was added)×2.1 was defined as the serum antibody titer. Sera were diluted in 100 folds initially. If the antibody titer cannot be measured at the 100-fold dilution, the serum antibody titer was set to 1:50.

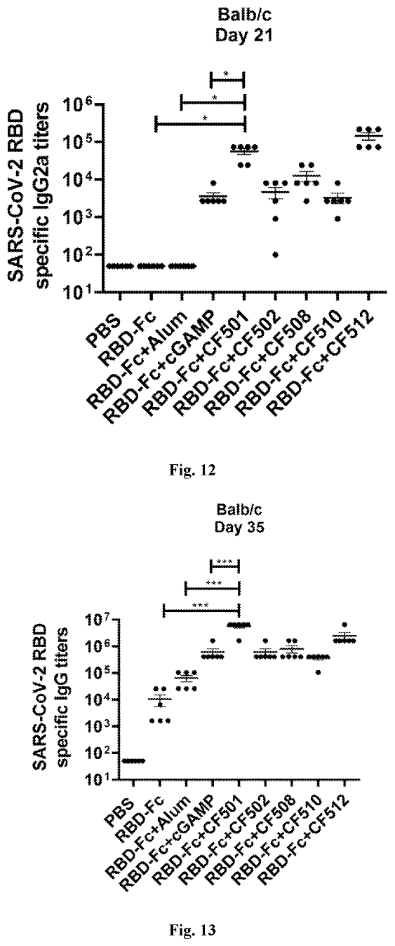

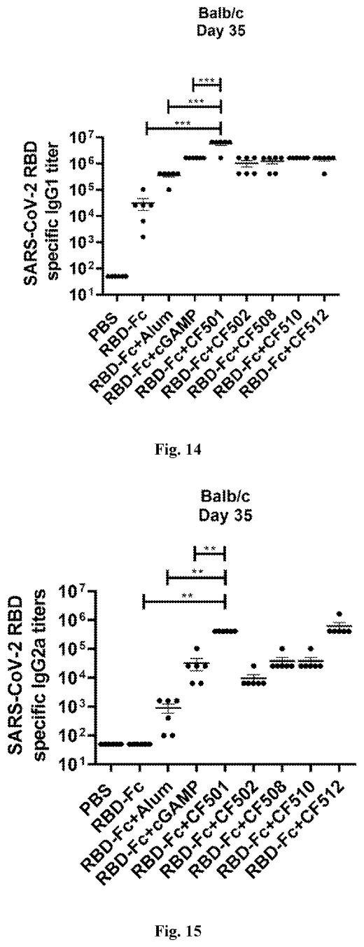

As shown in FIGS. 10-15, mice in each group can produce RBD-specific antibodies at a certain level at Days 21 and 35. It can be seen from the results of total mouse IgG at Day 21 or Day 35 that the RBD-specific antibody titers in the mice vaccinated with the SARS-CoV-2 RBD-Fc protein mixed with CF501 or CF512 are highest, and are significantly higher than those produced in the mice vaccinated with the RBD-Fc protein but without an adjuvant, the mice vaccinated with the SARS-CoV-2 RBD-Fc protein mixed with the aluminum adjuvant, and the mice vaccinated with the SARS-CoV-2 RBD-Fc protein mixed with cGAMP (Table 1 and Table 2). It can be seen from the results of the mouse IgG1 that, consistent with the results of the total IgG, the mice vaccinated with the SARS-CoV-2 RBD-Fc protein mixed with CF501 or CF512 produce the highest antibody titers (Table 3 and Table 4). It can be seen from the results of the mouse IgG2a that the RBD-Fc protein without an adjuvant and the RBD-Fc protein with the aluminum adjuvant cannot effectively activate the production of the RBD-specific IgG2a antibodies. The STING agonists CF501 and CF512 can potently activate the production of the RBD-specific IgG2a antibodies in vaccinated mice, which is significantly higher than that in the mice vaccinated with the SARS-CoV-2 RBD-Fc protein mixed with cGAMP (Table and Table 6). IgG1 represents the TH2 immune response, and IgG2a represents the TH1 immune response. The type TH1 immune response is essential for the protective effect of a vaccine. Therefore, the STING agonists CF501 and CF512 can strongly activate and enhance the type TH1 immune response.

TABLE 1
SARS-CoV-2 RBD-specific IgG antibody titers in the sera at 21 days after the vaccination of mice

| | | | RBD-Fc + | | |
|---|---|---|---|---|---|
| Group of | | | aluminium | | |
| mice | PBS | RBD-Fc | adjuvant | RBD-Fc + cGAMP | RBD-Fc + CF501 |
| RBD specific | 50 ± 0 | 633.3 ± 436.0 | 19800 ± 11103 | 97200 ± 24300 | 194400 ± 24300 |
| antibody titer | | | | | |
| Significance | P < 0.0001 | P < 0.0001 | P < 0.0001 | P = 0.0023 | |
| analysis | | | | | |
| (Compared with | | | | | |
| RBD-Fc + | | | | | |
| CF501) | | | | | |

The titer value in the table is the mean±Standard Error of Mean; significance analysis: compared with the CF501 treatment group (RBD-Fc+CF501), One-way ANOVA. The values in the following tables are expressed in the same way.

TABLE 2
SARS-CoV-2 RBD-specific IgG antibody titers in the sera at 35 days after the vaccination of mice

| | | | RBD-Fc + | | |
|---|---|---|---|---|---|
| Group of | | | aluminium | | |
| mice | PBS | RBD-Fc | adjuvant | RBD-Fc + cGAMP | RBD-Fc + CF501 |
| RBD specific | 50 ± 0 | 10400 ± 4866 | 64000 ± 17173 | 614400 ± 204800 | 5734400 ± 819200 |
| antibody titer | | | | | |
| Significance | P < 0.0001 | P < 0.0001 | P < 0.0001 | P < 0.0001 | |
| analysis(Compared | | | | | |
| with | | | | | |
| RBD-Fc + | | | | | |
| CF501) | | | | | |

TABLE 3
SARS-CoV-2 RBD-specific IgG1 antibody titers in the sera at 21 days after the vaccination of mice

| | | | RBD-Fc + | | |
|---|---|---|---|---|---|
| Group of | | | aluminium | | |
| mice | PBS | RBD-Fc | adjuvant | RBD-Fc + cGAMP | RBD-Fc + CF501 |
| RBD specific | 66.67 ± 10.54 | 20400 ± 10963 | 81000 ± 29205 | 656100 ± 146708 | 3061800 ± 922696 |
| antibody titer | | | | | |
| Significance | P < 0.0005 | P < 0.0006 | P < 0.0007 | P = 0.0083 | |
| analysis | | | | | |
| (Compared with | | | | | |
| RBD-Fc + | | | | | |
| CF501) | | | | | |

TABLE 4
SARS-CoV-2 RBD-specific IgG1 antibody titers in the sera at 35 days after the vaccination of mice

| | | | RBD-Fc + | | |
|---|---|---|---|---|---|
| Group of | | | aluminium | | |
| mice | PBS | RBD-Fc | adjuvant | RBD-Fc + cGAMP | RBD-Fc + CF501 |
| RBD specific | 50.00 ± 0 | 31200 ± 14894 | 358400 ± 51200 | 1638400 ± 0 | 5734400 ± 819200 |
| antibody titer | | | | | |
| Significance | P < 0.0001 | P < 0.0001 | P < 0.0001 | P < 0.0001 | |
| analysis | | | | | |
| (Compared with | | | | | |
| RBD-Fc + | | | | | |
| CF501) | | | | | |

TABLE 5
SARS-CoV-2 RBD-specific IgG2a antibody titers in
the sera at 21 days after the vaccination of mice

| | | | RBD-Fc + | | |
|---|---|---|---|---|---|
| Group of | | | aluminium | | |
| mice | PBS | RBD-Fc | adjuvant | RBD-Fc + cGAMP | RBD-Fc + CF501 |
| RBD specific | 50 ± 0 | 50 ± 0 | 50 ± 0 | 3600 ± 900.0 | 56700 ± 10246 |
| antibody titer | | | | | |
| Significance | P = 0.0023 | P = 0.0023 | P = 0.0023 | P = 0.0029 | |
| analysis | | | | | |
| (Compared with | | | | | |
| RBD-Fc + | | | | | |
| CF501) | | | | | |

TABLE 6
SARS-CoV-2 RBD-specific IgG2a antibody titers in
the sera at 35 days after the vaccination of mice

| | | | RBD-Fc + | | |
|---|---|---|---|---|---|
| Group of | | | aluminium | | |
| mice | PBS | RBD-Fc | adjuvant | RBD-Fc + cGAMP | RBD-Fc + CF501 |
| RBD specific | 50 ± 0 | 50 ± 0 | 900.0 ± 316.2 | 32000 ± 14594 | 409600 ± 0 |
| antibody titer | | | | | |
| Significance | P = 0.0003 | P = 0.0003 | P = 0.0003 | P = 0.0005 | |
| analysis | | | | | |
| (Compared with | | | | | |
| RBD-Fc + | | | | | |
| CF501) | | | | | |

Compared with the aluminum adjuvant, the STING agonists CF501, CF502, CF508, CF510, or CF512 can significantly improve the TH1 and TH2 immune responses in mice. Compared with other adjuvants, CF501 can significantly improve the TH1 and TH2 immune responses in mice.

### Example 3: Detection of the Cellular Immune Response in Mice Activated by CF501

1. Materials

6-week-old SPF Balb/c mice were purchased from Beijing Vital River Laboratory Animal Technology Co. Ltd., and the ELISPOT kits for mouse IFNγ, TNFα and IL-4 were purchased from Mabtech. The RBD full-length scanning peptide library is synthesized by GL Biochem (Shanghai) Ltd. Mouse IL-2 was purchased from Beijing Dakewe.

2. Experimental Steps:
(1) The mice were vaccinated with the same vaccination dose and procedure as in Example 2.
(2) The mice were euthanized at Day 7 after the third immunization to collect the spleens and lungs of the mice.
(3) The spleens and lungs of the mice were ground to prepare a spleen cell suspension and a lung cell suspension.
(4) The red blood cell lysis solution was used to lyse the red blood cells in the cell suspensions.
(5) The number of cells was counted after the cells were washed several times with PBS.
(6) The ELISPOT plate was taken out aseptically, to which the RPMI 1640 Medium with 10% FBS was added and it was blocked at 37° C. for 2 hrs.
(7) 2×10⁵ cells were added to each well of the ELISPOT plate, and 2 μg/ml of the RBD full-length scanning peptide library was added to each well simultaneously.
(8) The plate was incubated for 48 hrs in a 37° C. cell incubator, the cell supernatant was removed and the plate was washed 5 times with PBS.
(9) The biotinylated antibodies to IFNγ, TNFα and IL-4 were added respectively, and the plate was incubated for 2 hrs at room temperature.
(10) The plate was washed 5 times with PBS, and the ALP-conjugated avidin in the kit was added to the plate which was then placed at room temperature for 1 hr.
(11) The plate was washed 5 times with PBS, and the substrate in the kit was added for color development.
(12) After obvious spots appeared, the liquid was discarded and the plate was rinsed to stop the reaction.
(13) The ELSPOT reader was used to count the spots.

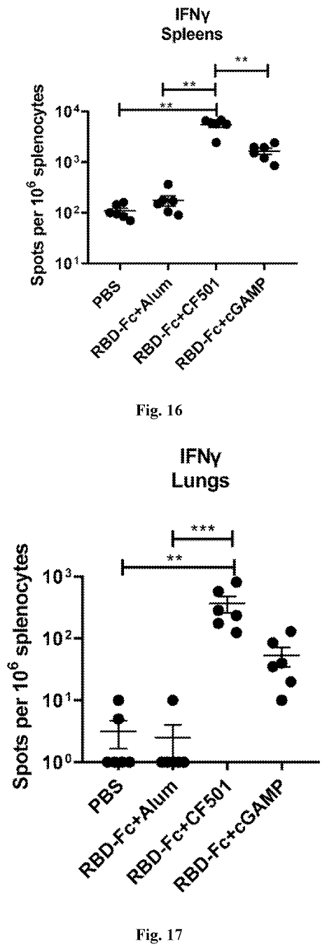

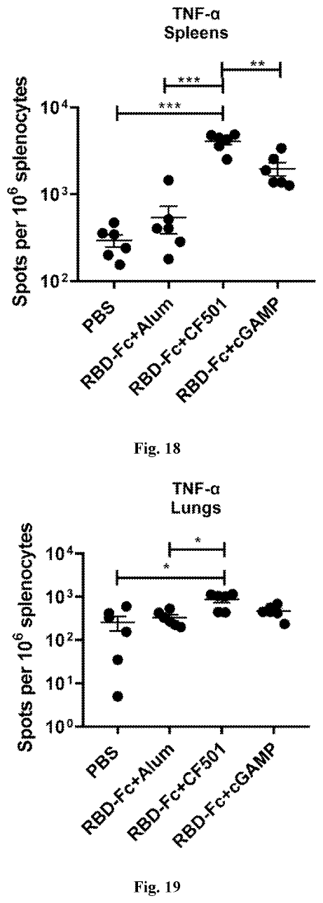

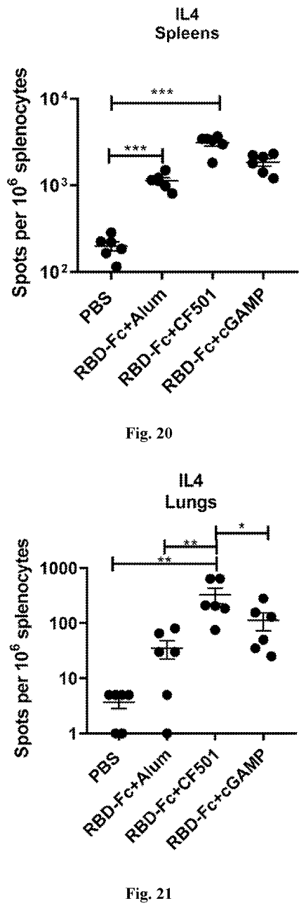

The results are shown in FIGS. 16-21. CF501 can strongly activate the T cell immune response in mice. IFNγ and TNFα represent the type of TH1 immune response, and IL-4 represents the type of TH2 immune response. In the splenocytes of the mice vaccinated with the RBD-Fc protein and the aluminum adjuvant, IFNγ and TNFα are almost not produced, but IL-4 can be significantly produced, indicating that the aluminum adjuvant can only activate the TH2 immune response. CF501 can strongly activate the TH1 immune response, and the levels of IFNγ and TNFα produced in spleen cells and lung cells are high in the mice vaccinated with the RBD-Fc protein mixed with CF501, which are significantly higher than those in the mice vaccinated with the RBD-Fc protein mixed with cGAMP. This indicates that CF501 is better than cGAMP in activating the TH1-biased cellular immune response. Compared with other adjuvants, CF501 can activate the cellular immune response in mice strongly.

### Example 4: The STING Agonists can Induce Potent Neutralizing Antibody Responses in Mice

1. Detection of the Neutralizing Antibody Levels in Mouse Sera Using a SARS-CoV-2 Pseudovirus Detection System.
(1) Production of the SARS-CoV-2 pseudovirus. HEK293T cells (purchased from the American Type Culture Collection, ATCC) were cotransfected with the constructed PcDNA3.1-SARS-CoV-2-S plasmid (provided by BEI Resources, US, catalog number NR-52420) and the HIV backbone plasmid (pNL4-3.Luc.R-E, from NIH AIDS Reagent Program, US, catalog number: 3418), and the cell supernatant (containing SARS-CoV-2 pseudovirus) was collected after 48 hrs.
(2) The collected sera from the vaccinated mice were inactivated at 56° C. for 30 min.
(3) Huh-7 cells (available from ATCC) were plated to a plate with 1×10⁴ cells per well.
(4) After 8 hrs, the sera were diluted with DMEM in 3 or 4 folds, and then the same volume of the SARS-CoV-2 pseudovirus was added. The mixture of the sera and the pseudovirus was incubated at 37° C. for 30 min.
(5) The mixture of sera and pseudovirus was added to Huh-7 cells (1×10⁴ cells/well).
(6) The plate was incubated for 12 hrs, and the culture medium was exchanged with a fresh DMEM medium containing 2% FBS.
(7) At 48 hrs after the medium exchange, a lysis solution (Promega, luciferase assay kit) was added to lyse the cells for 30 min, and then a substrate for the luciferase was added.
(8) A microplate reader was used to detect the luciferase value.
(9) The virus inhibition percentage and NT50 (the serum dilution at which 50% of the viruses was neutralized) were calculated.

The sera were diluted in the highest dilution, 100 folds initially. If the sera could not neutralize 50/of the viruses when the sera were diluted in 100 folds, the NT50 of the sera was 50 by default.

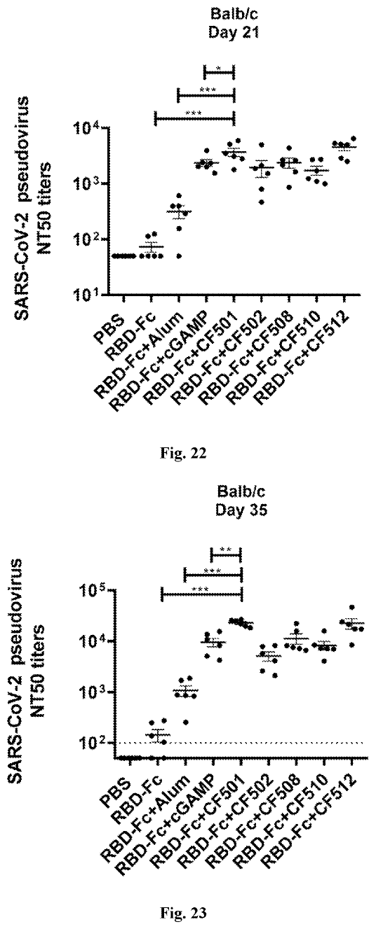

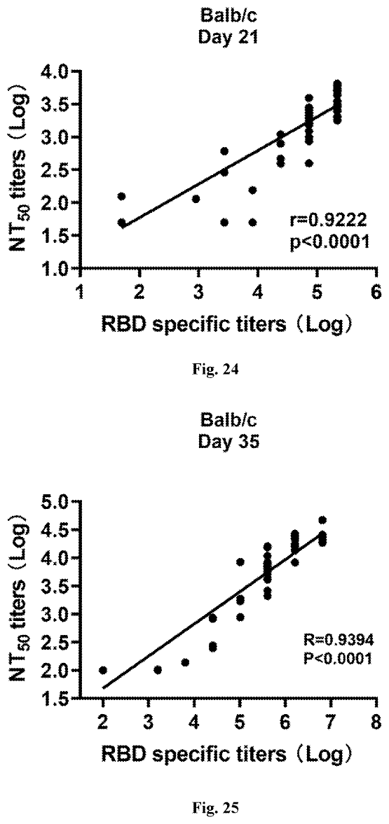

The neutralizing titers of sera collected at Day 21 against the SARS-CoV-2 pseudovirus are shown in FIG. 22 and Table 7. The neutralizing antibody titers of mice vaccinated with the RBD-Fc protein mixed with the CF501 or CF512 are highest, which are significantly higher than those of the mice vaccinated with the RBD-Fc protein but no adjuvant, the mice vaccinated with the RBD-Fc protein mixed with the aluminum adjuvant and the mice vaccinated with the RBD-Fc protein mixed with cGAMP. The neutralizing titers of sera collected at Day 35 against the SARS-CoV-2 pseudovirus are shown in FIG. 23 and Table 8. The average neutralizing titer of the sera from the mice vaccinated with the RBD-Fc protein mixed with the CF501 reaches 26730, while the average neutralizing titer of the sera from the mice vaccinated with the RBD-Fc protein mixed with the aluminum adjuvant is only 863. The neutralizing antibody titer in the mice vaccinated with the RBD-Fc protein mixed with the adjuvant CF501 is also significantly higher than the neutralizing antibody titer in the mice vaccinated with the RBD-Fc protein mixed with cGAMP, which shows that the neutralizing antibodies are strongly produced when CF501 is used as the adjuvant of the RBD-Fc protein. The neutralizing antibody titers in these vaccinated mice show a strong correlation with the RBD-specific IgG antibody titers, as shown in FIG. 24 and FIG. 25.

TABLE 7
Detection of neutralizing antibody titers against the SARS-CoV-2
pseudovirus in sera at 21 days after the vaccination of the mice

| | | | RBD-Fc + | | |
|---|---|---|---|---|---|
| Group of | | | aluminium | | |
| mice | PBS | RBD-Fc | adjuvant | RBD-Fc + cGAMP | RBD-Fc + CF501 |
| Neutralizing | 50 ± 0 | 73.17 ± 14.72 | 315.5 ± 80.64 | 2367 ± 333.7 | 3705 ± 631.2 |
| antibody titer | | | | | |
| Significance | P < 0.0001 | P < 0.0001 | P < 0.0001 | P = 0.0055 | |
| analysis | | | | | |
| (Compared with | | | | | |
| RBD-Fc + | | | | | |
| CF501) | | | | | |

TABLE 8
Detection of neutralizing antibody titers against the SARS-CoV-2
pseudovirus in sera at 35 days after the vaccination of the mice

| | | | RBD-Fc + | | |
|---|---|---|---|---|---|
| Group of mice | PBS | RBD-Fc | aluminium adjuvant | RBD-Fc + cGAMP | RBD-Fc + CF501 |
| neutralizing | 50 ± 0 | 143.5 ± 39.54 | 1067 ± 247.8 | 9595 ± 1847 | 22879 ± 1380 |
| antibody titer | | | | | |
| Significance analysis | P < 0.0001 | P < 0.0001 | P < 0.0001 | P < 0.0001 | |
| (Compared with | | | | | |
| RBD-Fc + CF501) | | | | | |

2. Plaque Reduction Method for the Detection of the Inhibitory Activity Against the Live SARS-CoV-2 Virus of the Sera.
(1) Vero-E6 (available from ATCC) cells were plated into a 96-well plate with 15,000 cells per well and incubated.
(2) After 24 hrs, the sera from each group of the mice were mixed. Then DMEM was used to dilute the sera in 3 or 4 folds serially.
(3) About 30 PFU of SARS-CoV-2 (SH-01, performed in P3 laboratory of Fudan University) was added and incubated with an equal volume of diluted sera for 30 min.
(4) The mixture was added to Vero-E6 cells (2×10⁴ cells/well) which were incubated for 2 hrs, then 100 μl of carboxymethyl cellulose was added.
(5) The plate was incubated for 48 hrs, and the supernatant was removed. 50 μl of 4% paraformaldehyde was added for fixation, and 50 μl of 1% crystal violet was added for staining.
(6) The plate was rinsed with tap water, and the plaques were counted.

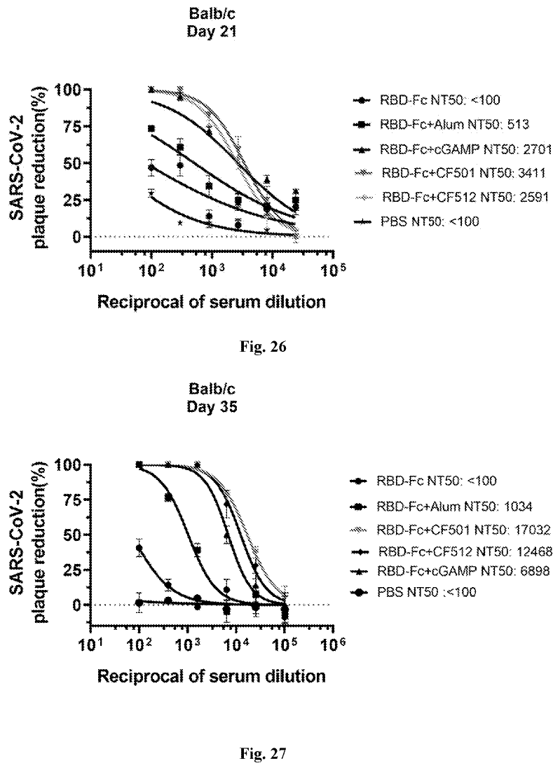

The inhibitory activities against the live SARS-CoV-2 virus of the sera from the vaccinated mice at Day 21 are shown in FIG. 26. The NT50 against the live SARS-CoV-2 virus in the mice vaccinated with the RBD-Fc protein mixed with the CF501 is 3411, while the NT50 in the mice vaccinated with the RBD-Fc protein mixed with the aluminum adjuvant is only 513. The NT50 in the mice vaccinated with the RBD-Fc protein mixed with cGAMP is 2701.

The inhibitory activities against the live SARS-CoV-2 virus of the sera from the vaccinated mice at Day 35 are shown in FIG. 27. The NT50 against the live SARS-CoV-2 virus in the mice vaccinated with the RBD-Fc protein mixed with CF501 is 17032, while the NT50 in the mice vaccinated with the RBD-Fc protein mixed with the aluminum adjuvant is 1032. The NT50 in the mice vaccinated with the RBD-Fc protein mixed with the cGAMP group is 6898.

3. Immunofluorescence Method for the Detection of the Inhibitory Activities Against the Live SARS-CoV-2 Virus of the Sera from the Vaccinated Mice
(1) Vero-E6 cells were plated into a 96-well plate with 10,000 cells per well.
(2) After 24 hrs, DMEM was used to dilute the mixed sera for each group (the sera from each group of the mice were mixed together) serially in 3 or 4 folds.
(3) An equal volume (60 μl) of the SARS-CoV-2 virus (1×10 PFU/ml) was mixed with the diluted sera (60 μl) and incubated for 30 min, which then was added to the cells.
(4) After 48 hrs, the cell supernatant was removed and 50 μl of 4% paraformaldehyde was added for fixation.
(5) 50 μl of 0.2% Trition was added for perforation.
(6) The rabbit anti-SARS-CoV-2 N protein antibody (Sino biological, 1:4000 dilution) was added to the plate which was then incubated at 37° C. for 1 hr.
(7) After the plate was washed 5 times with PBST, the fluorescently labelled donkey anti-rabbit IgG antibody conjugated with fluorescein flour488 (Thermo, 1:3000 dilution), was added and the plate was incubated for 1 hr.
(8) After the plate was washed 5 times with PBST, a fluorescence microscope was used to take pictures.

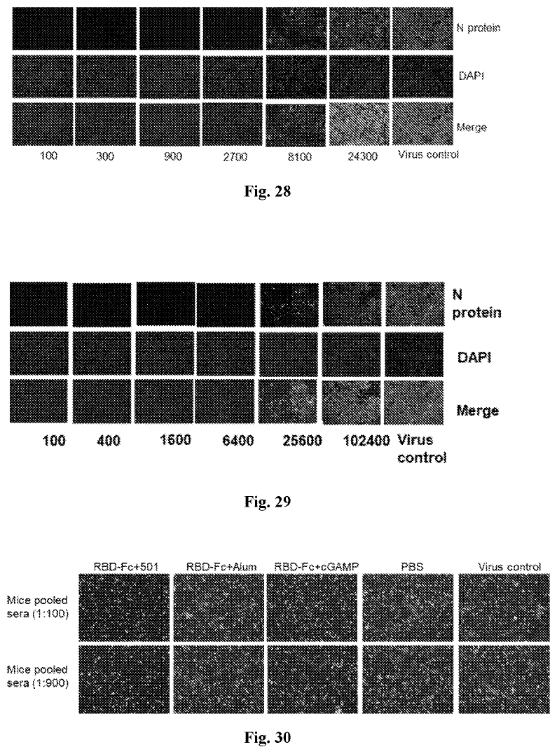

The results are shown in FIGS. 28 and 29. The sera from the mice vaccinated with the RBD-Fc protein mixed with CF501 at Day 21 when diluted in 2700 folds can effectively inhibit the expression of the SARS-CoV-2 N protein. The sera from the mice vaccinated with the RBD-Fc protein mixed with CF501 at Day 35 when diluted in 25600 folds can still effectively inhibit the expression of the SARS-CoV-2 N protein.

4. Inhibition of the SARS-CoV-2 S-Mediated Cell-Cell Fusion by the Sera
(1) HEK-293T cells (available from ATCC) were plated into 6-well plate.
(2) After 24 hrs of incubation, Vigofect transfection reagent was used to transfect the cells with PAAV-SARS-CoV-2-S-GFP plasmid (which was obtained by inserting the S protein gene of SARS-CoV-2 into the plasmid pAAV-IRES-EGFP; constructed by the inventor's laboratory). The plasmid can express the SARS-CoV-2 S protein on the surface of HEK293T cells after transfection.
(3) At 24 hrs after transfection, the cells were digested after the GFP fluorescence was fully expressed.
(4) Huh-7 cells were plated to a plate with 20,000 cells per well and incubated for 1 day.
(5) 60 μl of 293T cells expressing the SARS-CoV-2 S protein was mixed with an equal volume of the sera at each dilution and incubated for 30 min, which was then added to Huh-7 cells.
(6) After 6 hrs of incubation, when the cells fusion in the control well, that is, the cell well without the serum was obvious, paraformaldehyde was added to stop the fusion.
(7) The fused cells were observed under a fluorescence microscope.

The results are shown in FIG. 30. When the sera from mice vaccinated with the RBD-Fc protein mixed with CF501 were diluted in 900 folds, the SARS-CoV-2 S protein-mediated cell-cell fusion was substantially inhibited, while the sera from mice vaccinated with the RBD-Fc protein mixed with the aluminum adjuvant and the sera from mice vaccinated with the RBD-Fc protein mixed with cGAMP, when diluted in 900 folds, could not effectively inhibit SARS-CoV-2 S protein-mediated cell-cell fusion, as in the case of the sera from the mice treated with PBS. All these results indicate that the STING agonists of the present disclosure, especially CF501, can effectively activate the cellular immune response and humoral immune response in the mice compared with the aluminum adjuvant and cGAMP. The level of neutralizing antibodies in the vaccinated mice can be significantly increased when CF501 is used as the adjuvant for the RBD-Fc protein and a strong effect against the SARS-CoV-2 infection can be seen. Compared with other adjuvants, the neutralizing antibodies in the vaccinated mice can be strongly induced when CF501 is used as the adjuvant for the RBD-Fc protein.

### Example 5: Evaluation of the Cross-Neutralizing Activities Against SARS-Related Coronavirus in Sera from Mice Vaccinated with the RBD-Fc Protein Mixed with Different STING Agonists

1. Detection of the Cross-Binding Capacities to SARS-CoV RBD for Sera from Mice in the Example 2
(1) The SARS-CoV RBD-His (Kactus Biosystems Co. Ltd, catalog number: COV-VM4BD) protein was coated onto an ELISA plate overnight at 4° C.
(2) PBS containing 5% skimmed milk powder was used to block the ELISA plate.
(3) The mouse sera from Example 2 were serially diluted and added to the ELISA plate, which was then incubated at 37° C. for 30 min.
(4) The ELISA plate was washed 5 times with PBST, and the HRP-labeled rabbit anti-mouse IgG secondary antibody (Dako, 1:2000 dilution) was added.
(5) The plate was incubated at 37° C. for 30 min and washed 5 times with PBST.
(6) TMB substrate (Sigma) was added for color development and then H₂SO₄ was added to stop the reaction.
(7) The OD450 was detected with a microplate reader.

The highest dilution at which the OD450 is greater than the OD450 of the blank control group (no serum but PBS was added)×2.1 was defined as the serum antibody titer. Sera were diluted in 100 folds initially. If the OD450 at the 100-fold dilution was still not greater than 2.1 times that of the OD450 value of the blank control group, the serum antibody titer was set to 1:50.

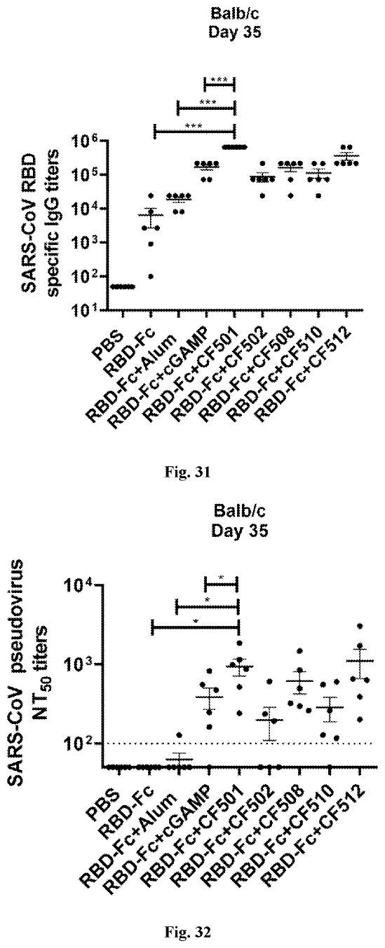

The results are shown in FIG. 31 and Table 9. The sera from mice vaccinated with the SARS-CoV-2 RBD-Fc protein could also show binding capacity to the RBD protein of SARS-CoV. Among the mice, the group of mice vaccinated with the RBD-Fc protein mixed with CF501 produces the most potent cross-binding antibodies. And the cross-binding antibody titers in the mice treated with the RBD-Fc protein and CF501 are significantly higher than those in the mice vaccinated with the RBD-Fc protein and the aluminum adjuvant or cGAMP.

TABLE 9
Detection of cross-neutralizing antibodies against the SARS-CoV RBD
protein in sera from vaccinated mice at Day 35 after vaccination

| | | | RBD-Fc + | | |
|---|---|---|---|---|---|
| Group of | | | aluminium | RBD-Fc + | |
| mice | PBS | RBD-Fc | adjuvant | cGAMP | RBD-Fc + CF501 |
| RBD specific | 50 ± 0 | 6467 ± 3744 | 18900 ± 3415 | 170100 ± 30737 | 656100 ± 0 |
| antibody titer | | | | | |
| Significance analysis | P < 0.0001 | P < 0.0001 | P < 0.0001 | P < 0.0001 | |
| (Compared with | | | | | |
| RBD-Fc + CF501) | | | | | |

2. Detection of the Neutralization Activities Against the SARS-CoV Pseudovirus in Sera from Vaccinated Mice
(1) Production of the SARS-CoV pseudovirus. HEK-293T cells were co-transfected with the plasmid PcDNA3.1-SARS-CoV-S (gifted by Dr. Du Lanying, New York Blood Center, USA; constructed and preserved by the inventor's laboratory) and the HIV backbone plasmid PNL-4-3-luc (provided by the NIH AIDS Research and Reference Reagent Program, catalog number 3418, owned by inventor's laboratory). The cell supernatant collected after 48 hrs contained the SARS-CoV pseudovirus.
(2) Huh-7 cells were plated into a plate with 10,000 cells per well.
(3) After 8 hrs of incubation, DMEM was used to dilute the sera in 3 folds, and the same volume of the SARS-CoV pseudovirus was added and the mixture was incubated at 37° C. for 30 min.
(4) The mixture of sera and the SARS-CoV pseudovirus were added to Huh-7 cells (1×10⁴ cells/well). The culture medium was exchanged with a fresh DMEM medium after incubation for 12 hrs.
(5) After 48 hrs of incubation, the cell lysis solution in the Promega luciferase kit was used to lyse the cells and detect the luciferase activity in the lysate.

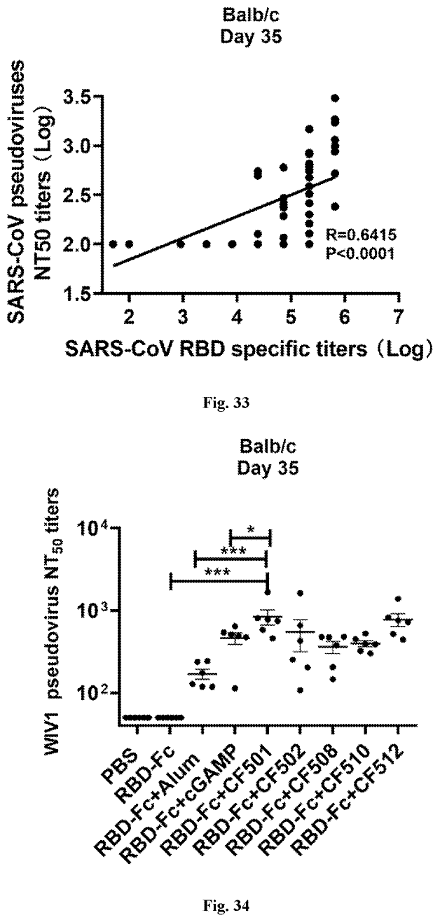

The results are shown in FIG. 32 and Table 10. Sera from mice vaccinated with the SARS-CoV-2 RBD-Fc protein or the SARS-CoV-2 RBD-Fc protein mixed with the aluminum adjuvant show almost no neutralization activities against the SARS-CoV pseudovirus. In contrast, sera from mice vaccinated with the SARS-CoV-2 RBD-Fc protein mixed with CF501 could produce significant cross-neutralization activities against the SARS-CoV pseudovirus. The average neutralizing antibody titer in the CF501-treated mice group is about 1000, which is significantly higher than the cross-neutralizing antibody titer produced by the cGAMP-treated mice group. In addition, we find that the neutralizing antibody titer produced by these mice against SARS-CoV pseudovirus infections correlates with the titers of SARS-CoV RBD specific antibodies at a certain degree (FIG. 33).

TABLE 10
Detection of neutralizing antibody titers against SARS-CoV in sera at Day
35 after the vaccination of mice

| | | | RBD-Fc + | | |
|---|---|---|---|---|---|
| Group of | | | aluminium | RBD-Fc + | |
| mice | PBS | RBD-Fc | adjuvant | cGAMP | RBD-Fc + CF501 |
| Neutralizing | 50 ± 0 | 50 ± 0 | 62.83 ± 12.83 | 384.5 ± 116.6 | 937.2 ± 227.1 |
| antibody titer | | | | | |
| Significance | P = 0.0020 | P = 0.0020 | P = 0.0020 | P = 0.0224 | |
| analysis | | | | | |

3. Detection of the Neutralization Activities Against Bat-Derived SARS-Like Viruses WIV1 and Rs3367 for the Sera from the Vaccinated Mice.
(1) Production of WIV1 and Rs3367 pseudoviruses. HEK293T cells were co-transfected with the plasmid PcDNA3.1-WIV1-S or PcDNA3.1-Rs3367-S (which is obtained by inserting the gene sequence of the S protein of WIV1 or Rs3367 into the PcDNA3.1 vector, respectively; the two plasmids were constructed by the inventor's laboratory) and the HIV backbone pNL4-3Luc.RE (from NIH AIDS Research and Reference Reagent Program, catalog number: 3418), and the cell supernatant (containing WIV1 or Rs3367 pseudovirus) was collected after incubation for 48 hrs.
(2) Huh-7 cells were plated into a plate with 10,000 cells per well.
(3) DMEM was used to dilute the sera in 3 folds serially, and then an equal volume of WIV1 pseudovirus or Rs3367 pseudovirus was added respectively. After incubation at 37° C. for 30 min, the sera and the pseudovirus were added to Huh-7 cells (1×10⁴ cells/well).
(4) The culture medium was exchanged with a fresh DMEM medium after incubation for 12 hrs.
(5) After 48 hrs of incubation, the cell lysis solution in the Promega luciferase kit was used to lyse the cells and detect the luciferase activity in the lysate.

The results are shown in FIG. 34, Table 11-1, and Table 11-2. The sera from mice vaccinated with the SARS-CoV-2 RBD-Fc protein alone show no inhibitory activities against the WIV1 pseudovirus, and the sera from mice vaccinated with the SARS-CoV-2 RBD-Fc protein mixed with the aluminum adjuvant show only weak inhibitory activities against the WIV1 pseudovirus with the average neutralizing antibody titer of 170. The sera from mice vaccinated with the SARS-CoV-2 RBD-Fc protein mixed with CF501 showed strong cross-neutralization activities against the WIV1 pseudovirus with the avenge neutralizing titer of 838, which is significantly higher than the titers of cross-neutralizing antibodies produced by the mice treated with cGAMP as an adjuvant.

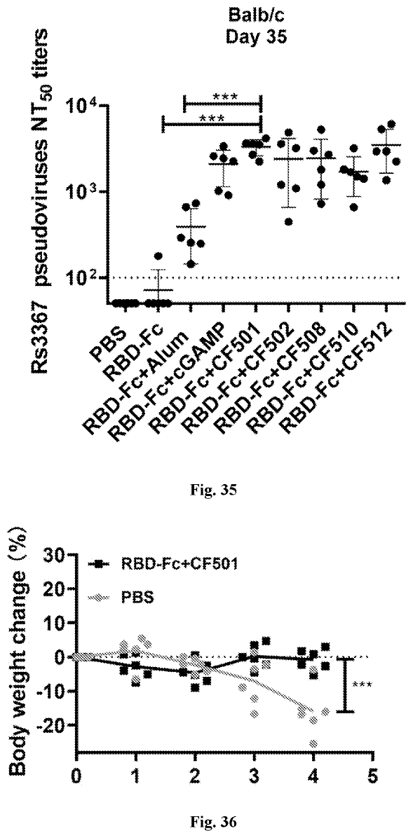

The results for the cross-inhibitory activities against the Rs3367 pseudovirus are consistent with the trend observed for the WIV1 pseudovirus (FIG. 35 and Table 11-2). The sera from mice vaccinated with the SARS-CoV-2 RBD-Fc protein alone show almost no detectable inhibitory activities against the Rs3367 pseudovirus. The sera from mice vaccinated with the SARS-CoV-2 RBD-Fc protein and the aluminum adjuvant show weak cross-neutralization activities against the Rs3367 pseudovirus with the average neutralizing antibody titer of 387. The sera from mice vaccinated with the SARS-CoV2 RBD-Fc protein mixed with CF501 show strong cross-neutralization activities against the Rs3367 pseudovirus with the average neutralizing titer of 3308, which is also significantly higher than the titers of cross-neutralizing antibodies produced by the mice treated with cGAMP. Little or no broadly neutralizing antibodies against SARS-related viruses could be induced in vaccinated mice when the aluminum adjuvant is used. Compared to the aluminum adjuvant, the STING agonist CF501 could be used as an adjuvant to induce cross-neutralizing antibodies against SARS-related viruses in mice potently.

TABLE 11-1
Detection of neutralizing antibody titers against the SARS-related virus
WIV1 pseudovirus in sera at Day 35 post the vaccination of the mice

| | | | RBD-Fc + | | |
|---|---|---|---|---|---|
| Group of | | | aluminium | RBD-Fc + | RBD-Fc + |
| mice | PBS | RBD-Fc | adjuvant | cGAMP | CF501 |
| Neutralizing | 50 ± 0 | 50 ± 0 | 170.0 ± 23.84 | 460.5 ± 73.74 | 838.5 ± 173.6 |
| antibody titer | | | | | |
| Significance analysis | P < 0.0001 | P < 0.0001 | P < 0.0001 | P = 0.0074 | |
| (Compared with | | | | | |
| RBD-Fc + CF501) | | | | | |

TABLE 11-2
Detection of neutralizing antibody titers against the SARS-related virus Rs3367
pseudovirus in sera at Day 35 after the vaccination of the mice

| | | | RBD-Fc + | | |
|---|---|---|---|---|---|
| Group of | | | aluminium | RBD-Fc + | RBD-Fc + |
| mice | PBS | RBD-Fc | adjuvant | cGAMP | CF501 |
| Neutralizing | 50 ± 0 | 71.33 ± 21.33 | 387.9 ± 99.74 | 2093 ± 389.4 | 3308 ± 288.2 |
| antibody titer | | | | | |
| Significance | P < 0.0001 | P < 0.0001 | P < 0.0001 | P = 0.0552 | |
| analysis | | | | | |

### Example 6: Challenge Test for the Human ACE2 Transgenic Mice Vaccinated with the RBD-Fc Protein Mixed with STING Agonist CF501

1. Materials: 8-Week-Old SPF ACE2 Transgenic Mice were Purchased from the Shanghai Model Organisms Center, Inc.
(1) 12 ACE2 transgenic mice were randomly divided into two groups of 6 mice each.
(2) In Group 1, the mice were injected intramuscularly with the SARS-CoV-2 RBD-Fc protein and CF501 according to the vaccination dose and vaccination procedures of Example 2. In Group 2, the mice were given an equal volume of PBS.
(3) Two weeks after the third immunization, the mice were challenged with the SARS-CoV-2 virus (1×10⁶ PFU) by nasal drip.
(4) The weight change of the mice was recorded daily after the challenge.
(5) At Day 4 after the challenge, the mice were sacrificed and their lungs, intestines and brains were taken.
(6) Trizol (Takara) was used to extract RNA from the tissues, and then the RT-qPCR detection kit (Takara) was used to detect the viral load in the mouse tissues.

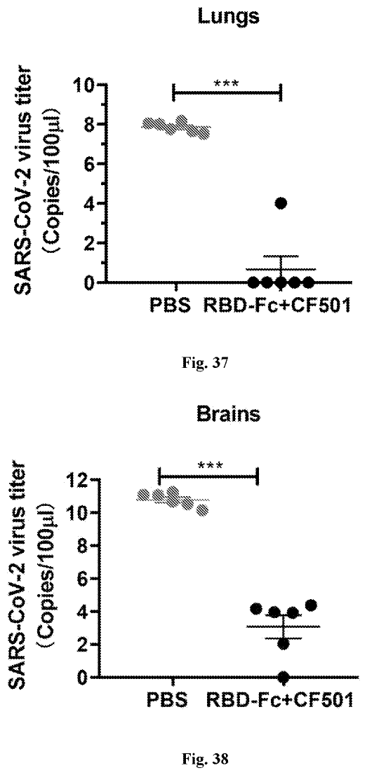

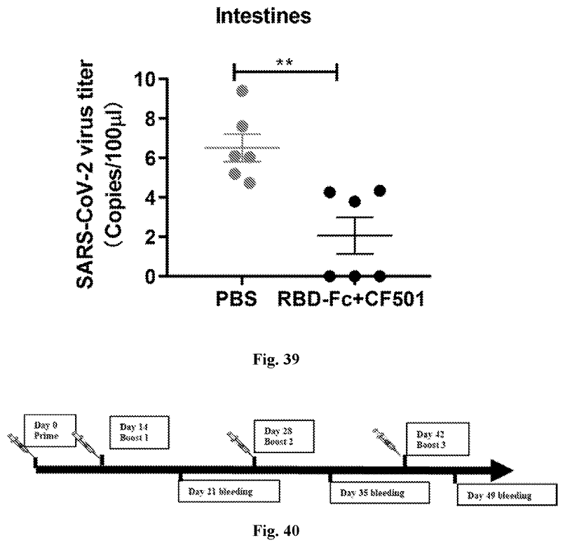

The results are shown in FIG. 36. After the challenge, no body weight loss was observed for the mice vaccinated with the RBD-Fc protein mixed with CF501. While more severe body weight loss in the mice treated with PBS were observed. As for detection of the viral load in the lungs, a significantly higher viral load (10⁸ copies/ml) in the lungs from mice treated with PBS was detected. In contrast, among the six mice treated with CF501, no viral load were tested in the lungs from the five mice. Only one lung could be detected the SARS-CoV-2 N gene with the titers of 10⁴ (FIG. 37). Similarly, a significantly higher viral load was detected in the brains in the mice treated with PBS reaching 10¹⁰, while the viral load in the brains of mice vaccinated with the STING agonist and the RBD-Fc protein was only about 10³ (FIG. 38). In the intestines of mice, the viral load in the intestines of the PBS-treated mice reaches 10⁶. In the 6 mice vaccinated with the STING agonists CF501 and the RBD-Fc protein, the N gene of the virus was not detected in 3 mice and a low viral load was detected in the other 3 mice (FIG. 39).

These results fully indicate that the vaccination of mice with CF501 and the RBD-Fc protein can effectively protect the mice from the SARS-CoV-2 infection.

### Example 7: Detection of the Activation of Immune Response by the STING Agonists in New Zealand White Rabbit Animal Model

1. Experimental Materials New Zealand White Rabbits were purchased from Shanghai Zeyu Biological Technology Co, Ltd.

2. Experimental Method

2.1 The Procedure for the Vaccination of the New Zealand White Rabbits is Shown in FIG. 40. The Steps are as Follows.
(1) 54 New Zealand white rabbits were divided into 9 groups of 6 rabbits each randomly,
(2) Group 1 was vaccinated with 10 μg of the RBD-Fc protein. Group 2 was vaccinated with 10 μg of the RBD-Fc protein and an equal volume of aluminum adjuvant. Group 3 was vaccinated with 10 μg of the RBD-Fc protein and 40 μg of CF501. Group 4 was vaccinated with 10 μg of the RBD-Fc protein and 40 μg of the STING agonist CF502. Group 5 was vaccinated with 10 μg of the RBD-Fc protein and 40 μg of the STING agonist CF508. Group 6 was vaccinated with 10 μg of the RBD-Fc protein and 40 μg of the STING agonist CF510. Group 7 was vaccinated with 10 μg of the RBD-Fc protein and 40 μg of the STING agonist CF512. Group 8 was vaccinated with 10 μg of the RBD-Fc protein and 40 μg of cGAMP Group 9 was injected with an equal volume of PBS.
(3) The New Zealand white rabbits were vaccinated at Days 1, 14, 28, and 42, and the rabbit sera were collected at Days 21, 35, and 49.

2.2 Evaluation of the Antibody Immune Response of the New Zealand White Rabbits after Vaccination.
(1) The obtained sera were inactivated at 56° C. for 30 mins.
(2) The RBD-His protein of SARS-CoV-2 (Kactus Biosystems Co. Ltd, catalog number: COV-VM4BD) was coated onto an ELISA plate overnight at 4° C.
(3) PBS containing 5% skimmed milk powder was used to block the plate for 2 hrs.
(4) PBST was used to dilute the rabbit sera in 3 or 4 folds and the diluted sera were added to the ELISA plate, which was incubated at 37° C. for 30 min.
(5) The ELISA plate was washed 5 times with PBST.
(6) The HRP-labeled goat anti-rabbit IgG enzyme-labeled secondary antibody (Dako, 1:2000 dilution) was added.
(7) The ELISA plate was incubated at 37° C. for 30 min, and washed with PBST.
(8) TMB substrate (Sigma) was added for color development, and H₂SO₄ was added to stop the reaction.
(9) A microplate reader was used to detect the OD450. The highest dilution at which the OD450 is greater than the OD450 of the blank control group (no serum but PBS was added)×2.1 was defined as the serum antibody titer. Sera were diluted in 100 folds initially. If the OD450 at the 100-fold dilution was still not greater than 2.1 times of the OD450 value of the blank control group, the serum antibody titer was set to 1:50.

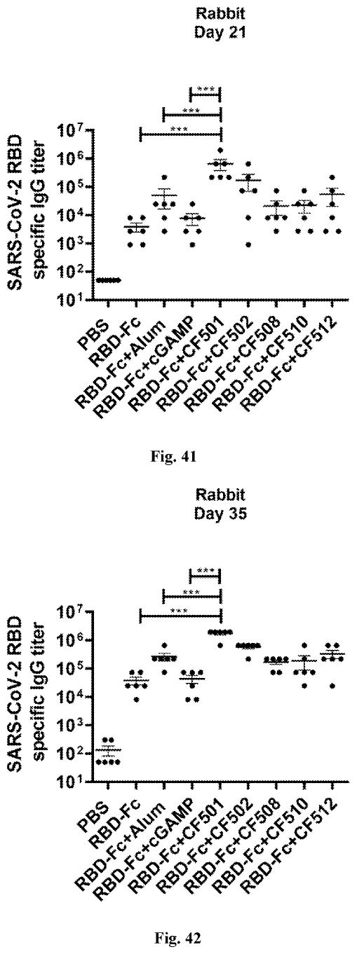

The results are shown in FIGS. 41 and 42 and Tables 12 and 13. At Day 21 after the rabbits were vaccinated, the sera from the rabbits vaccinated with the RBD-Fc protein mixed with CF501 show the highest antibody titers. The specific antibody titers against the SARS-CoV-2 RBD produced by the rabbits vaccinated with the RBD-Fc protein mixed with CF501 are significantly higher than those in the sera of the rabbits vaccinated with the RBD-Fc protein but no adjuvant, and the sera of rabbits vaccinated with the RBD-Fc protein mixed with the aluminum adjuvant or cGAMP.

At Day 35 after the vaccination, the titers of the SARS-CoV2 RBD-specific antibodies produced by the rabbits vaccinated with the RBD-Fc protein mixed with CF51 is still highest, and are significantly higher than those in the rabbits vaccinated with the RBD-Fc protein mixed with the aluminum adjuvant or cGAMP. It is worth noting that the other STING agonist such as CF512, CF510, CF508, and CF502 which showed similar structures to CF501 could not induce more potent binding antibodies relative to Alum in the rabbits, demonstrating that the minor change of the structure would significantly influence the adjuvant effects.

TABLE 12
Detection of specific antibody titers against SARS-CoV-2 RBD in sera at
Day 21 after the immunization of the rabbits

| | | | RBD-Fc + | | |
|---|---|---|---|---|---|
| | | | aluminium | RBD-Fc + | RBD-Fc + |
| Group of rabbits | PBS | RBD-Fc | adjuvant | cGAMP | CF501 |
| RBD specific | 50 ± 0 | 3900 ± 1368 | 50400 ± 33879 | 7800 ± 3522 | 656100 ± 276636 |
| antibody titer | | | | | |
| Significance analysis | P < 0.0001 | P < 0.0001 | P = 0.0001 | P < 0.0001 | |
| (Compared with | | | | | |
| RBD-Fc + CF501) | | | | | |

TABLE 13
Detection of specific antibody titers against SARS-CoV-2 RBD in sera at
Day 35 after the immunization of the rabbits

| | | | RBD-Fc + | | |
|---|---|---|---|---|---|
| | | | aluminium | RBD-Fc + | RBD-Fc + |
| Group of rabbits | PBS | RBD-Fc | adjuvant | cGAMP | CF501 |
| RBD specific | 133.3 ± 52.70 | 37800 ± 11391 | 267367 ± 81315 | 43200 ± 13500 | 1749600 ± 218700 |
| antibody titer | | | | | |
| Significance analysis | P < 0.0001 | P < 0.0001 | P < 0.0001 | P < 0.0001 | |
| (Compared with | | | | | |
| RBD-Fc + CF501) | | | | | |

### Example 8: Detection of Neutralizing Antibodies Against SARS-CoV-2 in Sera from Vaccinated Rabbits

1. A Method for the Detection of the Level of Neutralizing Antibodies in Sera Using the SARS-CoV-2 Pseudovirus
(1) The production of the SARS-CoV-2 pseudovirus was the same as in Example 4.
(2) Huh-7 cells were plated to a plate with 10,000 cells per well.
(3) After incubation for 8 hrs, DMEM was used to dilute the sera in 3 or 4 folds serially, to which the SARS-CoV-2 pseudovirus was added and incubated for 0.5 hrs.
(4) The mixture of the pseudovirus and sera was added to Huh-7 cells.
(5) After incubation for 12 hrs, the culture medium was exchanged with a fresh DMEM medium.
(6) After incubation for 48 hrs, the cells were lysed and the luciferase activity in the lysate was detected.

The sera were diluted in 100 folds initially. If the sera could not neutralize 50% of the viruses when the sera were diluted in 100 folds, the NT50 of the sera was 50 by default.

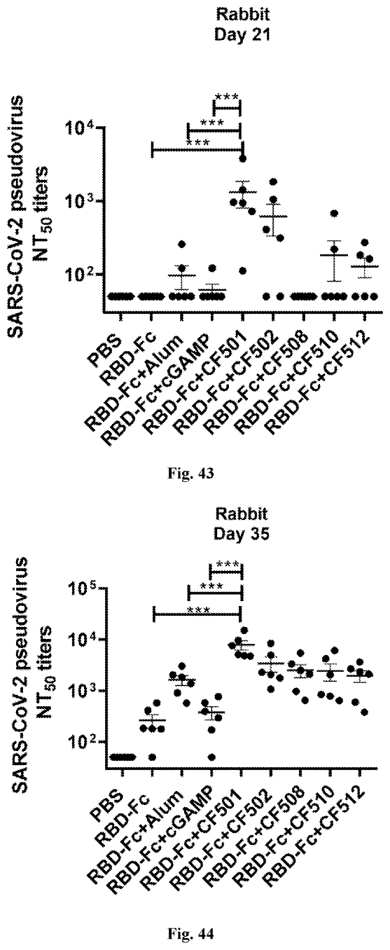

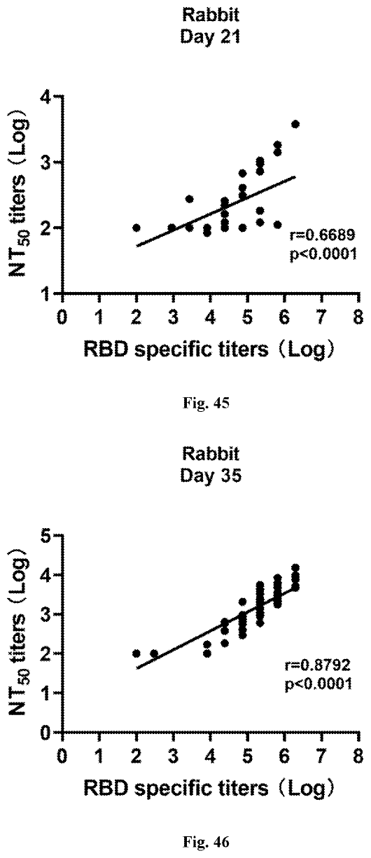

The results are shown in FIG. 43, FIG. 44, Table 14, and Table 15. FIG. 43 shows the neutralizing antibody titers against the SARS-CoV-2 pseudovirus at Day 21 after the vaccination of the rabbits. The neutralizing activities of the sera from the vaccinated rabbits are different from those of the sera from the vaccinated mice. It can be seen that the sera from the rabbits vaccinated with the RBD-Fc protein mixed with the aluminum adjuvant or cGAMP show only a low level of neutralizing antibodies after two immunizations, while the sera from the rabbit vaccinated with the RBD-Fc protein mixed with CF501 can still show a high level of neutralizing antibodies, indicating that the neutralizing antibody immune response is effectively activated in the rabbits vaccinated with the RBD-Fc protein and CF501. FIG. 44 shows the neutralizing antibody titers in sera at Day 35 after the first immunization. It can be seen that the levels of neutralizing antibodies produced by the rabbits vaccinated with the RBD-Fc protein mixed with cGAMP or with the RBD-Fc protein but no adjuvant are similar, indicating that cGAMP cannot activate the neutralizing antibody immune response in the vaccinated rabbits as strongly as in the vaccinated mice. The rabbits vaccinated with the RBD-Fc protein mixed with the aluminum adjuvant produce neutralizing antibodies at a certain level at Day 35, but the neutralizing antibody titers produced by the rabbits vaccinated with the RBD-Fc protein mixed with CF501 are significantly higher than those in the rabbits vaccinated with the RBD-Fc protein mixed with the aluminum adjuvant or other STING agonists. It is worth noting that the other STING agonist such as CF512, CF510, CF508, and CF502 which showed similar structures to CF501 could not induce more potent neutralizing antibodies relative to Alum in the rabbits, demonstrating that the minor change of the structure would significantly influence the adjuvant effects. The levels of neutralizing antibodies in sera from the vaccinated rabbits show a strong correlation with the RBD-specific IgG titers (FIG. 45 and FIG. 46). The rabbits vaccinated with the RBD-Fc protein mixed with the STING agonist CF501 as an adjuvant can produce the highest level of neutralizing antibodies.

TABLE 14
Detection of the neutralization activities against the SARS-CoV-2
pseudovirus in sera at Day 21 after the vaccination of the rabbits

| | | | RBD-Fc + | | |
|---|---|---|---|---|---|
| | | | aluminium | RBD-Fc + | RBD-Fc + |
| Group of rabbits | PBS | RBD-Fc | adjuvant | cGAMP | CF501 |
| Neutralizing | 50 ± 0 | 50 ± 0 | 96.50 ± 34.32 | 61.83 ± 11.83 | 1325 ± 522.8 |
| antibody titer | | | | | |
| Significance analysis | P = 0.0004 | P = 0.0004 | P = 0.0007 | P = 0.0005 | |
| (Compared with | | | | | |
| RBD-Fc + CF501) | | | | | |

TABLE 15
Detection of the neutralization activities against the SARS-CoV-2
pseudovirus in sera at Day 35 after the vaccination of the rabbits

| | | | RBD-Fc + | | |
|---|---|---|---|---|---|
| | | | aluminium | RBD-Fc + | RBD-Fc + |
| Group of rabbits | PBS | RBD-Fc | adjuvant | cGAMP | CF501 |
| Neutralizing | 50 ± 0 | 262.8 ± 78.38 | 1622 ± 358.2 | 375.3 ± 107.6 | 7843 ± 1663 |
| antibody titer | | | | | |
| Significance analysis | P < 0.0001 | P < 0.0001 | P < 0.0001 | P < 0.0001 | |
| (Compared with | | | | | |
| RBD-Fc + CF501) | | | | | |

2. Plaque Reduction Test for the Detection of the Inhibitory Activities Against the Live SARS-CoV-2 Virus of the Sera from the Vaccinated Rabbits
(1) Vero-E6 cells were plated into a 96-well plate with a total of 15,000 cells per well at 100 μl.
(2) The sera were diluted in 3 or 4 folds serially and incubated with about 30 PFU of the SARS-CoV-2 live virus for 30 min.
(3) 100 μl of the mixture of the virus and sera was added to the plated Vero-E6 cells and incubated for 2 hrs.
(4) 50 μl of carboxymethyl cellulose was added.
(5) After incubation for 48 hrs, 50 μl of paraformaldehyde was added for fixation. 50 μl of 1% crystal violet was added for staining.
(6) The plaques were counted.

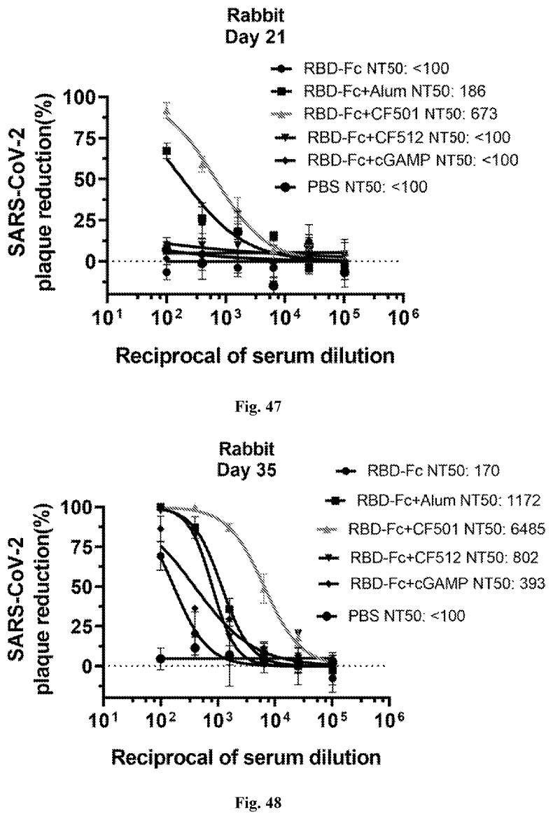

The results are shown in FIG. 47 and FIG. 48. FIG. 47 shows the inhibitory activities of the sera against the live SARS-CoV-2 virus at 21 days after the vaccination of the rabbits. The NT50 against SARS-CoV-2 of the rabbits vaccinated with the RBD-Fc protein mixed with the aluminum adjuvant was 183, and the NT50 of the sera from the rabbit vaccinated with the RBD-Fc protein mixed with CF501 was 673. FIG. 48 shows the inhibitory activities of the sera against the live SARS-CoV-2 virus at 35 days after the first immunization of the rabbits. The NT50 of the sera from the rabbit vaccinated with the RBD-Fc protein mixed with the aluminum adjuvant is 1172, while the NT50 of the sera from the rabbit vaccinated with the RBD-Fc protein mixed with cGAMP is 393. The NT50 of the sera from the rabbit vaccinated with the RBD-Fc protein mixed with CF501 is 6485, which is highest.

3. Immunofluorescence Test for the Detection of the Inhibitory Activities Against the SARS-CoV-2 Virus of Sera from the Vaccinated Rabbits
(1) Vero-E6 cells were plated into a 96-well plate with 10,000 cells per well.
(2) The sera from the rabbits vaccinated with the RBD-Fc protein mixed with the STING agonist 501 were diluted in 3 or 4 folds with DMEM. The diluted sera were incubated with the same volume of SARS-CoV-2 (1×10⁵ PFU/ml) at 37° C. for 30 min and the mixture was added to Vero-E6 cells.
(3) After incubation for 48 hrs, the cell supernatant was removed, and 4% paraformaldehyde was added for fixation. 0.02% Triton was added for perforation.
(4) The rabbit anti-SARS-CoV-2 N protein antibody (Sino biological, 1:3000 dilution) was added and the plate was incubated for 30 min.
(5) The fluorescein flour488-labeled goat anti-rabbit secondary antibody (Thermo, 1:3000 dilution) was added and the plate was incubated for 30 min.
(6) The plate was washed with PBST, and a fluorescence inverted microscope was used to take pictures.

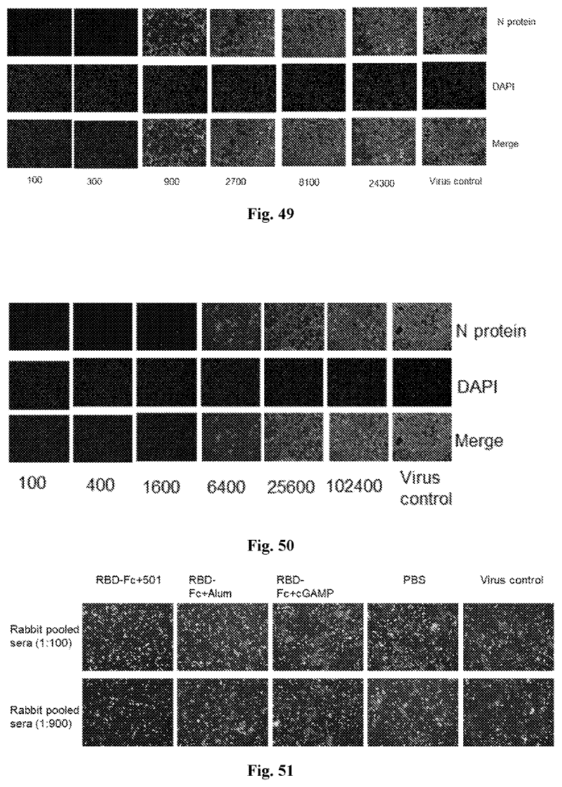

The results are shown in FIG. 49 and FIG. 50. The sera from the vaccinated rabbits at Day 21 when diluted in 900 folds can significantly inhibit the expression of the SARS-CoV-2 N protein. The sera from the vaccinated rabbits at Day 35 when diluted in 6400 folds can still effectively inhibit the expression of the SARS-CoV-2 N protein.

4. Inhibition of the SARS-CoV-2 S-Mediated Cell-Cell Fusion by the Sera
(1) The HEK-293T cells were transfected with the PAAV-SARS-CoV-2-S plasmid. After the appearance of the fluorescence, the cells were 293T cells expressing the SARS-CoV-2 S fluorescent protein.
(2) After incubation for 24 hrs, when the fluorescence became obvious, the cells were collected and used as effector cells.
(3) DMEM was used to dilute the rabbit sera in 3 folds serially and incubated with 293T cells expressing the SARS-CoV-2 S fluorescent protein.
(4) The sera and effector cells were added to the plated Huh-7 cells.
(5) After incubation for 6 hrs, when fused cells were formed (in the wells with no added serum, but only 293T cells expressing the SARS-CoV-2 S protein), paraformaldehyde was added for fixation and the fusion reaction was stopped.
(6) A fluorescence inverted microscope was used to take pictures.

The results are shown in FIG. 51. The sera from the rabbits vaccinated with the RBD-Fc protein mixed with CF501 when diluted in 900 folds can still effectively inhibit the SARS-CoV-2 S protein-mediated cell-cell fusion, while the sera from the rabbits vaccinated with the RBD-Fc protein mixed with the aluminum adjuvant or cGAMP as well as the sera from the control rabbits cannot effectively inhibit the SARS-CoV-2 S protein-mediated cell-cell fusion.

5. The Neutralization Activities Against SARS-CoV-2 of the Sera from the Vaccinated Rabbits after the Fourth Immunization

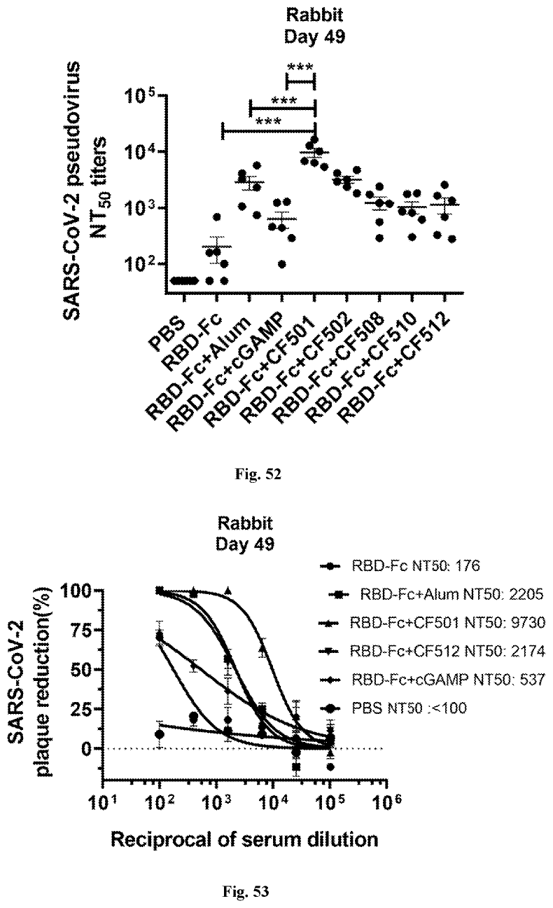

Sera were collected one week after the fourth immunization of the rabbits. The neutralization activities of these sera against SARS-CoV-2 were evaluated by the pseudovirus detection system. The results are shown in FIG. 52 and Table 16. As shown, after 4 immunizations, the neutralizing antibody titers produced by the rabbits vaccinated with the RBD-Fc protein mixed with CF501 is still highest, and the level of the neutralizing antibodies produced by the rabbits vaccinated with the RBD-Fc protein mixed with CF501 is still significantly higher than those in the rabbits vaccinated with the RBD-Fc protein mixed with aluminum adjuvant or cGAMP. The plaque reduction test was further used to detect the inhibition of the SARS-CoV-2 live virus by these sera, which shows that the NT50 of the sera from the rabbits vaccinated with the RBD-Fc protein mixed with CF501 is 9720, while the NT50 of the sera from the rabbits vaccinated with the RBD-Fc protein mixed with cGAMP is only 534. The NT50 of the sera from the rabbits vaccinated with the RBD-Fc protein mixed with the aluminum adjuvant is 2205 (FIG. 53).

All these results indicate that the most potent neutralizing antibody response can be produced when CF501 is used as an adjuvant of the RBD-Fc protein for the vaccination of rabbits. The immune response produced in the rabbits vaccinated with the RBD-Fc protein mixed with cGAMP is very week. Although the rabbits vaccinated with the RBD-Fc protein mixed with the aluminum adjuvant also produce neutralizing antibodies at a certain level, the level is still significantly lower than the level of neutralizing antibodies produced by the rabbits vaccinated with the RBD-Fc protein mixed with CF501. Therefore, the neutralizing antibodies can be produced most effectively in both the mice vaccinated with the RBD-Fc protein mixed with CF501 and the rabbits vaccinated with the RBD-Fc protein mixed with CF501 as compared with the mice or rabbits treated with other adjuvants.

TABLE 16
Detection of neutralization activities against the SARS-CoV-2 pseudovirus
in sera at 49 days after the vaccination of the rabbits

| | | | RBD-Fc + | | |
|---|---|---|---|---|---|
| Group of | | | aluminium | RBD-Fc + | RBD-Fc + |
| rabbits | PBS | RBD-Fc | adjuvant | cGAMP | CF501 |
| Neutralizing | 50 ± 0 | 202.1 ± 99.64 | 2858 ± 768.9 | 635.3 ± 205.1 | 9666 ± 1769 |
| antibody titer | | | | | |
| Significance | P < 0.0001 | P < 0.0001 | P < 0.0001 | P < 0.0001 | |
| analysis | | | | | |

### Example 9: Evaluation of the Broad-Spectrum Antiviral Property Against SARS-Related Viruses of Rabbit Anti-Sera

1. Packaging of SARS-Related Pseudoviruses (SARS-CoV, WIV1 and Rs3367).
(1) HEK-293T cells were co-transfected with the plasmid PCDNA-3.1-SARS-CoV-S, PCDN-3.1-WIV1-S or PCDNA-3.1-Rs3367-S (which is obtained by inserting the S gene sequence of SARS-CoV-2, WIV1 or Rs3367 into the PcDNA3.1 vector, respectively) and HIV backbone plasmid PNL-4-3-Luc plasmid.
(2) The cell supernatant containing the corresponding pseudovirus was collected after incubation for 48 hrs.

2. Evaluation of Inhibition of SARS-CoV, WIV1 and Rs3367 Pseudoviruses by Sera
(1) The sera of rabbits immunized for 3 times and 4 times were serially diluted with DMEM in 3 folds, and then incubated with an equal volume of the corresponding pseudovirus (SARS-CoV, WIV1 or Rs3367) at 37° C. for 30 mins.
(2) The mixture of the pseudovirus and sera was added to the Huh-7 cells which were already plated in a plate.
(3) After incubation for 12 hrs, the culture medium was exchanged with a fresh DMEM.
(4) After incubation for 48 hrs, the cell lysis solution in the luciferase detection kit from Promega was used to lyse the cells and the luciferase activity in the lysate was detected.

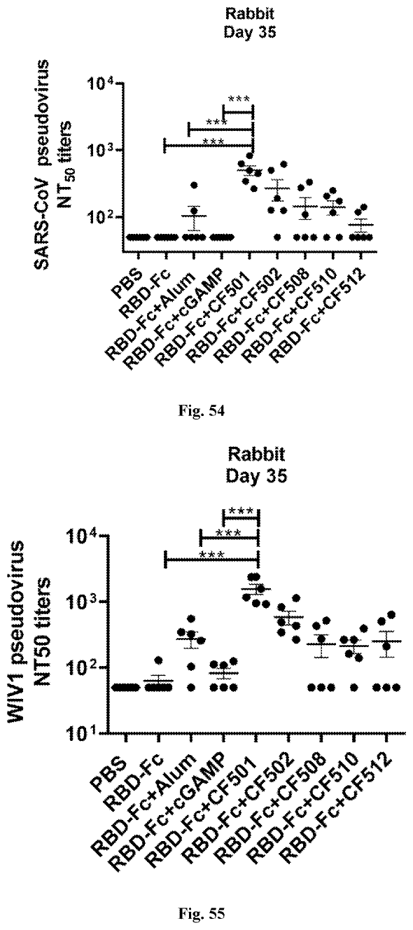

The results are shown in FIG. 54, FIG. 55, and Table 17. FIG. 54 shows the results of the neutralization titers against the SARS-CoV pseudovirus in sera at 35 days after vaccination of the rabbits. It can be seen that, for the sera from the rabbits vaccinated with the RBD-Fc protein but no an adjuvant or the RBD-Fc protein mixed with cGAMP, the cross-neutralization against the SARS-CoV pseudovirus is not detected at the highest dilution (1:100). The sera from the rabbits vaccinated with the RBD-Fc protein mixed with the aluminum adjuvant show an average neutralizing titer of 137 against the SARS-CoV pseudovirus. The sera from the rabbits vaccinated with the RBD-Fc protein mixed with the STING agonist CF501 show the highest cross-neutralizing antibody titer of 502 against the SARS-CoV pseudovirus, which is significantly higher than those in the sera from the rabbits vaccinated with the RBD-Fc protein mixed with the aluminum adjuvant or cGAMP. FIG. 55 and Table 18 show the results of the neutralization titer against the WIV1 pseudovirus in sera at 35 days after the vaccination of the rabbits. It can be seen that the sera from the rabbits vaccinated with the RBD-Fc protein but no an adjuvant only show a very weak cross-neutralization activity against the WIV1 pseudovirus with an average neutralization titer of 105. The sera from the rabbits vaccinated with the RBD-Fc protein mixed with cGAMP also show a very weak cross-neutralization activity with an average neutralization titer of 107. The sera from the rabbits vaccinated with the RBD-Fc protein mixed with the aluminum adjuvant show a weak neutralization activity against the WIV1 pseudovirus with an average neutralization titer of 280. In contrast, the sera from the rabbits vaccinated with the RBD-Fc protein mixed with CF501 show a very high level of cross-neutralizing antibodies, with an average neutralizing antibody titer of 1567.

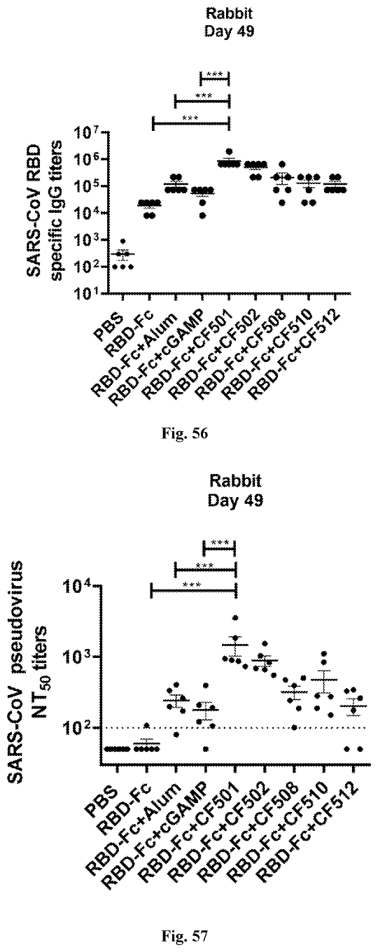

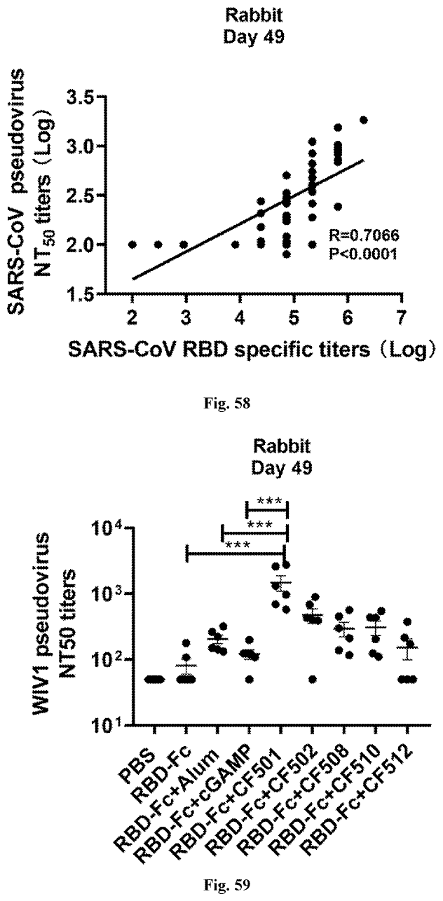

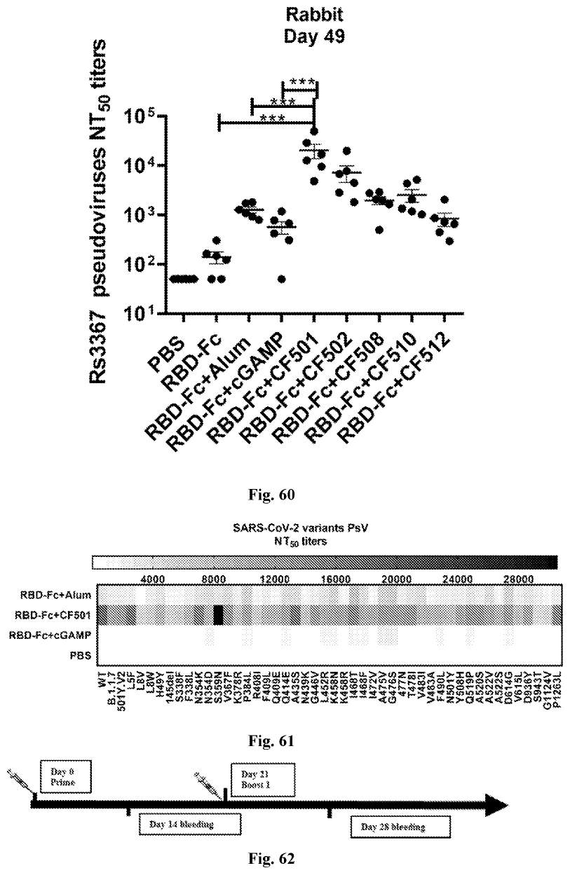

We further detected antibodies against the SARS-CoV RBD at 45 days after the vaccination of the rabbits, and found that the level of the SARS-CoV RBD specific antibodies in the sera from the rabbits vaccinated with the RBD-Fc protein mixed with CF501 is significantly higher than those in the sera from the rabbits vaccinated with the RBD-Fc protein mixed with cGAMP and from the rabbits vaccinated with the RBD-Fc protein mixed with the aluminum adjuvant (Table 19, FIG. 56). Through the pseudovirus detection system, it is found that the sera from the rabbits vaccinated with the RBD-Fc protein but no adjuvant still show no neutralization activity against the SARS-CoV pseudovirus at a dilution of 1:100, while the sera from the rabbits vaccinated with the RBD-Fc protein mixed with cGAMP show a weak cross-neutralization activity with an average neutralization titer of 178. The sera from the rabbits vaccinated with the RBD-Fc mixed with the aluminum adjuvant still show a weak cross-neutralizing antibody activity against SARS-CoV with an average neutralizing titer of 242. The titer of cross-neutralizing antibodies produced by the rabbits vaccinated with the RBD-Fc protein mixed with CF501 reaches 1472 (FIG. 57 and Table 20). A certain correlation between the SARS-CoV RBD-specific antibody titer and the neutralizing antibody titer is shown (FIG. 58). The neutralizing antibody titers of sera against pseudoviruses WIV1 and Rs3367 at 49 days after the vaccination of the rabbits are shown in FIG. 59 and Table 21. For the WIV1 pseudovirus, the neutralizing titer of sera from the rabbits vaccinated with the RBD-Fc protein mixed with CF501 is 1487, which is significantly higher than those of sera from the rabbits vaccinated with the RBD-Fc protein mixed with cGAMP and from the rabbits vaccinated with the RBD-Fc protein mixed with the aluminum adjuvant. For the Rs3367 pseudovirus, the neutralizing antibody titer of sera from the rabbits vaccinated with the RBD-Fc protein mixed with CF501 reaches 20299, which is significantly higher than those of sera from the rabbits vaccinated with the RBD-Fc protein mixed with cGAMP and from the rabbits vaccinated with the RBD-Fc protein mixed with the aluminum adjuvant (FIG. 60 and Table 22). CF501 when used as an adjuvant for a COVID-19 vaccine can potently activate a broad-spectrum immune response against SARS-related viruses in vaccinated rabbits.

TABLE 17
Detection of neutralizing activities against the SARS-CoV pseudovirus in
sera at Day 35 after the vaccination of rabbits

| | | | RBD-Fc+ | | |
|---|---|---|---|---|---|
| | | | aluminium | RBD-Fc + | RBD-Fc + |
| Group of rabbits | PBS | RBD-Fc | adjuvant | cGAMP | CF501 |
| Neutralizing | 50 ± 0 | 50 ± 0 | 103.9 ± 40.94 | 50.00 ± 0.00 | 501.8 ± 82.75 |
| antibody titer | | | | | |
| Significance analysis | P < 0.0001 | P < 0.0001 | P < 0.0001 | P < 0.0001 | |
| (Compared with | | | | | |
| RBD-Fc + CF501) | | | | | |

TABLE 18
Detection of neutralizing activities against the SARS-CoV related virus WIV1
pseudovirus in sera at Day 35 after the vaccination of rabbits

| | | | RBD-Fc + | | |
|---|---|---|---|---|---|
| | | | aluminium | RBD-Fc + | RBD-Fc + |
| Group of rabbits | PBS | RBD-Fc | adjuvant | cGAMP | CF501 |
| Neutralizing | 50 ± 0 | 63.20 ± 13.20 | 272.6 ± 74.28 | 82.53 ± 14.71 | 1567 ± 276.5 |
| antibody titer | | | | | |
| Significance analysis | P < 0.0001 | P < 0.0001 | P < 0.0001 | P < 0.0001 | |
| (Compared with | | | | | |
| RBD-Fc + CF501) | | | | | |

TABLE 19
The titers of the SARS-COV RBD specific antibody in sera at Day 49
after the vaccination of rabbits

| | | | RBD-Fc + | | |
|---|---|---|---|---|---|
| | | | aluminium | RBD-Fc + | RBD-Fc + |
| Group of rabbits | PBS | RBD-Fc | adjuvant | cGAMP | CF501 |
| RBD specific | 300 ± 126.5 | 18900 ± 3415 | 121500 ± 30737 | 54000 ± 12135 | 874800 ± 218700 |
| antibody titer | | | | | |
| Significance analysis | P < 0.0001 | P < 0.0001 | P < 0.0001 | P < 0.0001 | |
| (Compared with | | | | | |
| RBD-Fc + CF501) | | | | | |

TABLE 20
Detection of the neutralizing antibody titers against the SARS-CoV pseudovirus
in sera at Day 49 after the vaccination of rabbits

| | | | RBD-Fc + | | |
|---|---|---|---|---|---|
| | | | aluminium | RBD-Fc + | RBD-Fc + |
| Group of rabbits | PBS | RBD-Fc | adjuvant | cGAMP | CF501 |
| Neutralizing | 50 ± 0 | 59.67 ± 9.667 | 242.0 ± 47.74 | 178.8 ± 49.47 | 1472 ± 446.9 |
| antibody titer | | | | | |
| Significance analysis | P < 0.0001 | P < 0.0001 | P = 0.0002 | P < 0.0001 | |
| (Compared with | | | | | |
| RBD-Fc + CF501) | | | | | |

TABLE 21
Detection of the neutralizing antibody titers against the SARS-CoV related virus
WIV1 pseudovirus in sera at Day 49 after the vaccination of rabbits

| | | | RBD-Fc + | | |
|---|---|---|---|---|---|
| | | | aluminium | RBD-Fc + | RBD-Fc + |
| Group of rabbits | PBS | RBD-Fc | adjuvant | cGAMP | CF501 |
| Neutralizing | 500 | 81.08 ± 21.67 | 206.0 ± 31.50 | 121.2 ± 19.59 | 1487 ± 391.9 |
| antibody titer | | | | | |
| Significance | P < 0.0001 | P < 0.0001 | P < 0.0001 | P < 0.0001 | |
| analysis | | | | | |
| (Compared with | | | | | |
| RBD-Fc + CF501) | | | | | |

TABLE 22
Detection of the neutralizing antibody titers against the SARS-CoV related
virus Rs3367 pseudovirus in sera at Day 49 after the vaccination of rabbits

| | | | RBD-Fc + | | |
|---|---|---|---|---|---|
| Group of | | | aluminium | RBD-Fc + | RBD-Fc + |
| rabbits | PBS | RBD-Fc | adjuvant | cGAMP | CF501 |
| Neutralizing | 50 ± 0 | 139.1 ± 38.01 | 1266 ± 170.8 | 564.7 ± 160.2 | 20299 ± 6686 |
| antibody titer | | | | | |
| Significance | P < 0.0001 | P < 0.0001 | P < 0.0001 | P < 0.0001 | |
| analysis | | | | | |
| (Compared with | | | | | |
| RBD-Fc + CF501) | | | | | |

3. Detection of the Neutralization Activities of Antisera Against SARS-CoV-2 Mutant Viruses after the Vaccination of Rabbits

At present, mutations continue to occur in SARS-CoV-2. In order to verify whether the antibodies produced by the rabbits immunized with vaccines containing adjuvants can still have a strong neutralizing effect on the current SARS-CoV-2 mutant viruses, we conducted site-directed mutations on a plasmid of the wild-type SARS-CoV-2. Pseudoviruses of more than 40 currently discovered SARS-CoV-2 mutants were produced. For the rabbits vaccinated with the RBD-Fc protein mixed with the aluminum adjuvant, cGAMP or CF501, the neutralization activities against these mutant viruses in the sera from the rabbits were detected after 4 immunizations. The results are shown in FIG. 61. The sera of the rabbits vaccinated with the RBD-Fc protein mixed with CF501 show the highest neutralization activities against these pseudoviruses of the 40 SARS-CoV-2 mutants, with NT50 between 3323 and 14188. Although the sera from rabbits vaccinated with the RBD-Fc protein mixed with the aluminum adjuvant can also show neutralization activities against these mutant strains, the neutralization activities are weaker than those of the sera from the rabbits vaccinated with the RBD-Fc protein mixed with CF501. The activities of the sera from rabbits vaccinated with the RBD-Fc protein mixed with cGAMP are weakest.

These results indicate that the sera from the rabbits vaccinated with the RBD-Fc protein mixed with CF501 can show broad-spectrum and potent neutralization activities against SARS-CoV-2 mutants and SARS-related viruses.

### Example 10: Comparison of the Immune Response of Rhesus Monkeys Vaccinated with CF501 or the Aluminum Adjuvant Mixed with the SARS-CoV-2 RBD-Fc Protein

1. Materials

Nine 2-year-old rhesus monkeys were purchased from Beijing Xierxin Biological Co., Ltd, of which 5 were females and 4 were males.

2. Evaluation of the Humoral Immune Response after the Vaccination of Rhesus Monkeys with a Vaccine

2.1 The Vaccination Procedure of Rhesus Monkeys is Shown in FIG. 62, and the Specific Steps are as Follows:
(1) 9 rhesus monkeys were divided into 3 groups of 3 animals each randomly.
(2) Group 1 of the rhesus monkeys was vaccinated intramuscularly with 100 μg of the RBD-Fc protein and an equal volume of the aluminum adjuvant.
(3) Group 2 of the rhesus monkeys was vaccinated intramuscularly with 100 μg of the RBD-Fc protein and 400 μg of CF501.
(4) Group 3 of the rhesus monkeys was vaccinated intramuscularly with an equal volume of PBS.
(5) A booster immunization was performed on the rhesus monkeys at Day 21.
(6) Sera collected from the rhesus monkeys at Days 14 and 28 were evaluated for antibodies.

2.2 Detection of the SARS-CoV-2 RBD Specific Antibody Titers in Sera from the Vaccinated Rhesus Monkeys
(1) The SARS-CoV-2 RBD was coated onto an ELISA plate at 4° C. overnight.
(2) PBS containing 5% skimmed milk powder was added to block the plate at 37° C. for 2 hrs.
(3) PBST was used to dilute the sera in 3 or 4 folds serially and the diluted sera were added to the ELISA plate, which was then incubated at 37° C. for 30 min.
(4) The HRP-labeled goat anti-monkey IgG enzyme-labeled secondary antibody (Abcam, 1:10000 dilution) was added to the plate which was incubated at 37° C. for 30 min.
(5) After the plate was washed 5 times with PBST, the TMB substrate was added for color development and then H₂SO₄ was added to stop the reaction.
(6) The OD450 was read in a microplate reader.

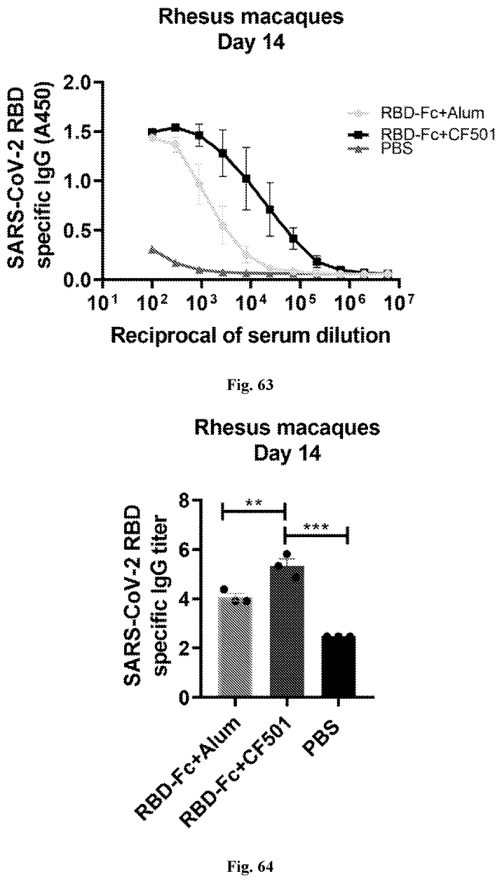

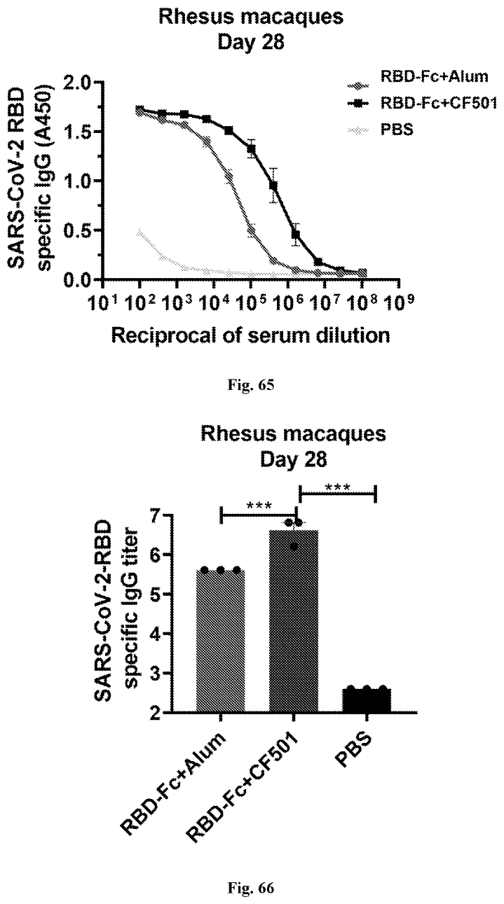

The results are shown in FIGS. 63-66 and Tables 23 and 24. After the first vaccination, the SARS-CoV-2 RBD specific antibody titer in the sera from the rhesus monkeys vaccinated with the RBD-Fc protein mixed with CF501 reaches 218776, which is significantly higher than those of the sera from the rhesus monkeys vaccinated with the RBD-Fc protein mixed with the aluminum adjuvant and the rhesus monkeys vaccinated with PBS. After the second vaccination, the SARS-CoV-2 RBD specific antibody titer in the sera from the rhesus monkeys vaccinated with the RBD-Fc protein mixed with CF501 reaches 4130475, which is also significantly higher than those of the sera from the rhesus monkeys vaccinated with the RBD-Fc protein mixed with the aluminum adjuvant and the rhesus monkeys vaccinated with PBS. This indicates that CF501 can still activate the humoral immune responses in primates as strongly as in mice and rabbits. As compared with the aluminum adjuvant, a potent antibody humoral immune response can be produced in vaccinated monkeys when CF501 is used as an adjuvant for a COVID-19 vaccine.

TABLE 23
Detection of the SARS-CoV-2 RBD specific antibody titers in
the sera at 14 days after the vaccination of rhesus monkeys

| | | RBD-Fc + | |
|---|---|---|---|
| Group of | | aluminium | RBD-Fc + |
| rhesus monkeys | PBS | adjuvant | CF501 |
| RBD specific | 300.0 ± 0 | 13500 ± 5400 | 315900 ± 175230 |
| antibody titer | | | |
| Significance | P < 0.0001 | P = 0.0009 | |
| analysis | | | |
| (Compared with | | | |
| RBD-Fc + CF501) | | | |

TABLE 24
Detection of the SARS-CoV-2 RBD specific antibody titers in
the sera at 28 days after the vaccination of rhesus monkeys

| | | RBD-Fc + | |
|---|---|---|---|
| Group of | | aluminium | RBD-Fc + |
| rhesus monkeys | PBS | adjuvant | CF501 |
| RBD specific | 400 ± 0 | 409600 ± 0 | 4915200 ± 1638400 |
| antibody titer | | | |
| Significance | P < 0.0001 | P = 0.0009 | |
| analysis | | | |
| (Compared with | | | |
| RBD-Fc + CF501) | | | |

### Example 11: Evaluation of the Cellular Immune Response in the Vaccinated Rhesus Monkeys

(1) Whole blood from the rhesus monkeys was collected at Week 1 of the first and second vaccinations with the RBD-Fc protein mixed with aluminum adjuvant or CF501 or with PBS.
(2) PBMC was isolated from the whole blood.
(3) The ELISPOT kit was used to detect the cellular immune response in the rhesus monkeys.

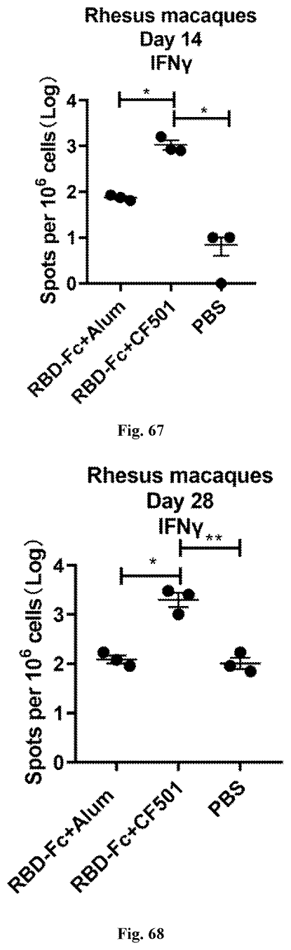

The results are shown in FIG. 67. After the first vaccination of the rhesus monkeys, CF501 can significantly activate the cellular immune response, and the level of IFN-γ produced by the rhesus monkeys vaccinated with the RBD-Fc protein mixed with CF501 is significantly higher than those produced by the rhesus monkeys vaccinated with the RBD-Fc protein mixed with the aluminum adjuvant and the rhesus monkeys vaccinated with PBS. The aluminum adjuvant cannot effectively activate the cellular immune response in the vaccinated rhesus monkeys. After the second vaccination of the rhesus monkeys, CF501 can further activate the cellular immune response in the vaccinated monkeys, while the aluminum adjuvant still does not effectively activate the cellular immune response in the vaccinated monkeys (FIG. 68). This indicates that CF501 can effectively activate both the humoral and cellular immune responses of rhesus monkeys after one vaccination. Compared with the aluminum adjuvant, a potent cellular immune response can be produced in the vaccinated monkeys when CF501 is used as an adjuvant for a COVID-19 vaccine to immunize monkeys.

### Example 12: Detection of the Neutralizing Antibody Titers in Sera from the Vaccinated Rhesus Monkeys

1. The SARS-CoV-2 Pseudovirus was Used to Detect Neutralizing Antibody Titers Against SARS-CoV-2 in Sera from the Vaccinated Rhesus Monkeys
(1) The production of SARS-CoV-2 pseudovirus was the same as in Example 4.
(2) Huh-7 cells were plated into a plate with 10,000 cells per well.
(3) After incubation for 8 hrs, DMEM was used to dilute the serum in 3 or 4 folds, and an equal volume of the SARS-CoV-2 pseudovirus was added. The mixture of the pseudovirus and the sera was incubated for 0.5 hrs.
(4) A total of 100 μl of the mixture of the pseudovirus and the sera was added to the plated Huh-7 cells.
(5) After incubation for 12 hrs, the culture medium was exchanged with a fresh DMEM medium.
(6) After incubation for 48 hrs, the cell lysis solution in the luciferase detection kit of Promega was used to lyse the cells and the luciferase activity in the lysate was detected.

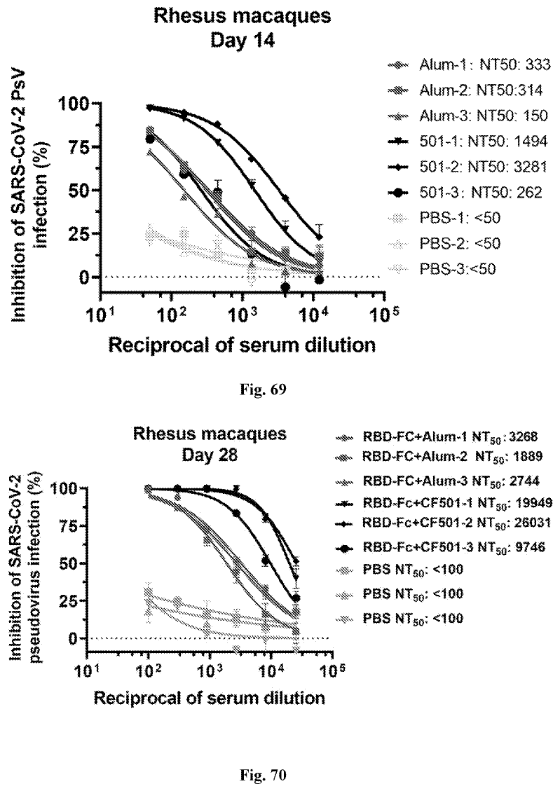

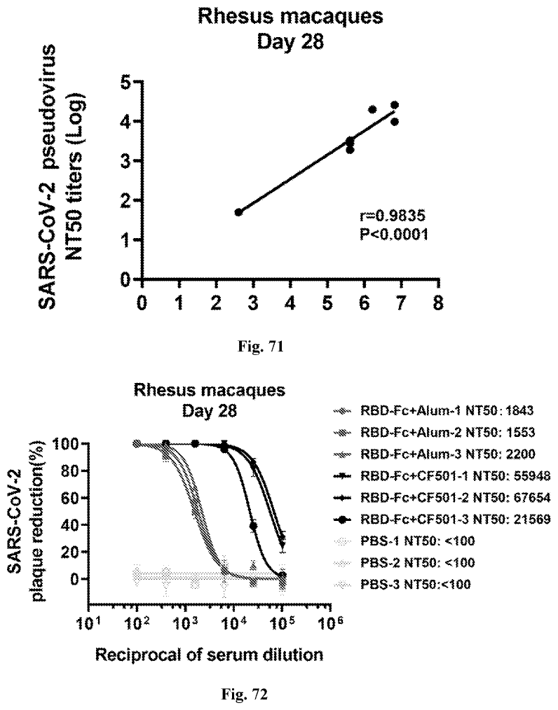

The results are shown in FIG. 69. After the first vaccination of the rhesus monkeys, the sera from the three rhesus monkeys vaccinated with the RBD-Fc protein mixed with CF501 show the NT50 against the SARS-CoV-2 pseudovirus of 1494, 3281 and 262, respectively, while the sera from rhesus monkeys vaccinated with the RBD-Fc protein mixed with the aluminum adjuvant show the NT50 against the SARS-CoV-2 pseudovirus of 333, 314 and 150, respectively. After the second vaccination of the rhesus monkeys, the sera from the three rhesus monkeys vaccinated with the RBD-Fc protein mixed with CF501 show the NT50 against the SARS-CoV-2 pseudovirus of 19949, 26031 and 9746, respectively. The sera from the three rhesus monkeys vaccinated with the RBD-Fc protein mixed with the aluminum adjuvant show the NT50 against the SARS-CoV-2 pseudovirus of 3268, 1889, and 2744, respectively (FIG. 70). We find that the level of the SARS-CoV-2 RBD specific antibodies in sera at Day 28 shows a very high correlation with the level of the neutralizing antibodies (FIG. 71).

2. The Plaque Reduction Test for the Detection of the Inhibitory Activity of Rhesus Monkey Sera Against the Live SARS-CoV-2 Virus
(1) Vero-E6 cells were plated into a 96-well plate with 15,000 cells per well.
(2) DMEM was used to dilute the serum in 4 folds serially, and incubated with about 30 PFU of the SARS-CoV-2 live virus for 30 mins.
(3) The mixture was added to Vero-E6 cells.
(4) 50 μl of carboxymethyl cellulose was added after incubation for 2 hrs.
(5) After incubation for 48 hrs, 50 μl of paraformaldehyde was added for fixation. 50 μl of 1% crystal violet was added for staining.
(6) The plaques were counted.

The results are shown in FIG. 72. The results of the plaque reduction test show that the sera from the three rhesus monkeys vaccinated with the RBD-Fc protein mixed with CF501 show the NT50 against the live SARS-CoV-2 virus of 55948, 67654, and 21569, respectively. The sera of the three rhesus monkeys vaccinated with the RBD-Fc protein mixed with the aluminum adjuvant show the NT50 against the live SARS-CoV-2 virus of 1843, 1553 and 2200, respectively. These results indicate that a high neutralizing antibody level can be induced when CF501 is used as an adjuvant for the RBD-Fc protein, and the neutralizing antibody level in the sera from the rhesus monkeys vaccinated with the RBD-Fc protein mixed with the CF501 is tens of times higher than that in the sera from the rhesus monkeys vaccinated with the RBD-Fc protein mixed with the aluminum adjuvant. As compared with the aluminum adjuvant, a more potent neutralizing antibody immune response can be produced in vaccinated monkeys when CF501 is used as an adjuvant for a COVID-19 vaccine.

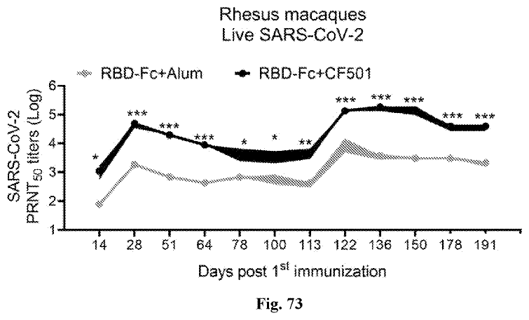

The neutralizing antibody titers in the sera were also continuously monitored after vaccination of the rhesus monkeys. It was found that the neutralizing antibody titer against the live SARS-CoV-2 virus could still reach 4696 at 113 days post the first vaccination in the sera from the rhesus monkeys immunized with CF501/RBD-Fc. In contrast, the neutralizing antibody titer against the live SARS-CoV-2 virus was only 439 at day 113 post the first vaccination in the sera from the rhesus monkeys immunized with Alum/RBD-Fc (FIG. 73).

The rhesus monkeys were vaccinated for the third time at 115 days after the first vaccination of the rhesus monkeys, and then the neutralizing antibody titers against the live SARS-CoV-2 virus in the sera were detected at 122 days after the first vaccination of the rhesus monkeys. It is found that the neutralizing antibody titer against the live SARS-CoV-2 virus in the sera from rhesus monkeys vaccinated with the RBD-Fc protein mixed with CF501 reaches 134827. In contrast, the neutralizing antibody titer against the live SARS-CoV-2 virus in the sera from rhesus monkeys vaccinated with the RBD-Fc protein mixed with the aluminum adjuvant reaches 9771. The neutralizing antibody titers in sera were continuously monitored by 191 days after the first vaccination of the rhesus monkeys. The neutralizing antibody titer against the live SARS-CoV-2 virus in the sera is 39746 even at Day 191 after the vaccination of the rhesus monkeys with the RBD-Fc protein mixed with CF501. In contrast, the neutralizing antibody titer against the live SARS-CoV-2 virus in the sera is 2153 after the vaccination of the rhesus monkeys with the RBD-Fc protein mixed with the aluminum adjuvant (FIG. 73). These data fully indicate the strong and lasting immune protection produced by the RBD-Fc protein mixed with CF501.

### Example 13: Evaluation of the Neutralization Activities Against SARS-CoV-2 Variants in Sera after Vaccination of Rhesus Monkeys

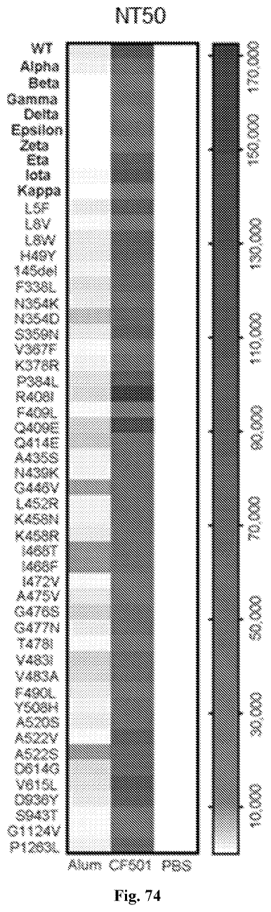

The pseudoviruses of SARS-CoV-2 variants or mutants were prepared as described in Example 9. The neutralization activities against pseudoviruses of 9 SARS-CoV-2 variants and 41 SARS-CoV-2 single-point mutants were detected in the sera at 28 days after vaccination of rhesus monkeys (at 7 days after the second vaccination). Results are shown in FIG. 74. The sera of rhesus monkeys immunized with the RBD-Fc protein mixed with CF501 can effectively neutralize 9 variants including Alpha, Beta, Gamma, Delta, Epsilon, Zeta, Eta, Iota and Kappa at 28 days after the vaccination. The neutralization titers against these mutants in the sera from the rhesus monkeys vaccinated with the RBD-Fc protein mixed with CF501 range from 29584 to 123589. In contrast, the neutralization titers against these mutants in the sera from the rhesus monkeys vaccinated with the RBD-Fc protein mixed with aluminum adjuvant only range from 242 to 2016. The sera from the rhesus monkeys vaccinated with the RBD-Fc protein mixed with CF501 can also effectively neutralize pseudoviruses of the 41 SARS-CoV-2 mutants with a single-point mutation (FIG. 74) (Table 25).

TABLE 25
Neutralizing antibody titers against pseudoviruses of SARS-
CoV-2 mutants in sera at 28 days after the vaccination of
the rhesus monkeys with the RBD-Fc protein mixed with CF501,
the RBD-Fc protein mixed with aluminum adjuvant, and PBS

| | RBD-Fc protein and | RBD-Fc protein | |
|---|---|---|---|
| | aluminum adjuvant | and CF501 | PBS |
| WT | 2016.709 | 123589.2 | 42 |
| Alpha | 1327.347 | 54590.9 | 30 |
| Beta | 527.7018 | 32191.73 | 51 |
| Gamma | 517.632 | 46899.58 | 30 |
| Delta | 512.9432 | 39991.92 | 30 |
| Epsilon | 555.5689 | 62069.41 | 30 |
| Zeta | 470.3765 | 29584.59 | 30 |
| Ela | 510.2304 | 85419.99 | 30 |
| Iota | 891.2509 | 106269.3 | 30 |
| Kappa | 242.5263 | 34804.65 | 30 |
| L5F | 1898.03 | 99743.25 | 30 |
| L8V | 1034.981 | 30398.55 | 30 |
| L8W | 2308.313 | 89462.56 | 82 |
| H49Y | 2308.313 | 89462.56 | 30 |
| 145del | 1462.58 | 65152.96 | 30 |
| F338L | 2860.815 | 67826.3 | 44 |
| N354K | 1812.266 | 59824.7 | 30 |
| N354D | 5122.726 | 73829.17 | 30 |
| S359N | 2249.084 | 86918.53 | 30 |
| V367F | 1018.095 | 29894.64 | 30 |
| K378R | 1308 | 48074 | 74 |
| P384L | 2837.482 | 104593.8 | 30 |
| R408I | 3114.383 | 172466.7 | 30 |
| Q409E | 4063.617 | 147728.8 | 30 |
| Q414E | 4428.612 | 58225.1 | 30 |
| A435S | 1575.46 | 62894.31 | 30 |
| N439K | 1330.701 | 55906.22 | 49 |
| G446V | 7504.668 | 62424.8 | 30 |
| L452R | 1526.859 | 53706.13 | 35 |
| K458N | 1761.519 | 59937.34 | 30 |
| K458R | 2072.309 | 48442.34 | 69 |
| I468F | 8487.73 | 46622.65 | 30 |
| I468T | 8369.784 | 49520.1 | 30 |
| I472V | 1096.021 | 38456.29 | 32 |
| A475V | 2464.519 | 41866.79 | 30 |
| G476S | 4082.423 | 54380.82 | 30 |
| S477N | 1802.13 | 62509.55 | 30 |
| T478I | 818.8348 | 35003.07 | 30 |
| V483A | 3962.196 | 61985.73 | 30 |
| V483I | 3343.511 | 62577.38 | 30 |
| F490L | 2139.793 | 39785.69 | 70 |
| Y508H | 1299.038 | 44675.63 | 30 |
| A520S | 1949.772 | 63373.74 | 30 |
| A522V | 1120.645 | 87125.2 | 72 |
| A522S | 8061.501 | 69374.29 | 30 |
| D614G | 3149.792 | 68386.77 | 30 |
| V615L | 2516.953 | 109446.8 | 30 |
| D936Y | 1862.12 | 87603.78 | 30 |
| S943T | 1000.155 | 33523.48 | 34 |
| G1124V | 1082.595 | 38951.63 | 30 |
| P1263L | 1617.196 | 72178.79 | 30 |

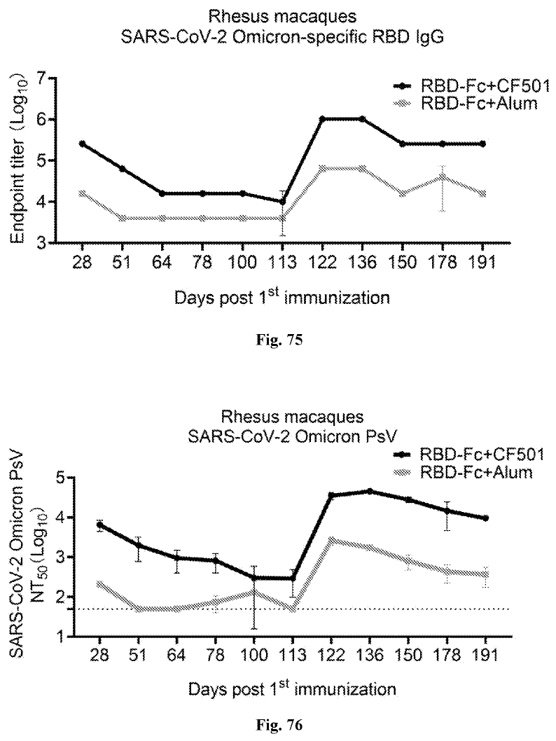

Recently, the Omicron variant becomes the main epidemic strain. Thus, the binding ability and the neutralizing antibody titers to the Omicron pseudovirus in the sera were also detected from 28 days to 191 days after the vaccination of the rhesus monkeys. The results are shown in FIG. 75 and FIG. 76. Antibodies specifically binding to the RBD of Omicron and neutralizing antibodies against the Omicron pseudovirus can still be effectively produced in the sera of the rhesus monkeys vaccinated with the RBD-Fc protein mixed with CF501. The neutralizing antibody titer against the Omicron pseudovirus in sera reached 6468 at 28 days after the vaccination of the rhesus monkeys with the RBD-Fc protein mixed with CF501. In contrast, the neutralizing antibody titer against the Omicron pseudovirus in sera is only 208 for the rhesus monkeys vaccinated with the RBD-Fc protein mixed with the aluminum adjuvant. The neutralizing antibody titer against Omicron pseudovirus in sera is 35066 at 7 days after the third vaccination of the rhesus monkeys with the RBD-Fc protein mixed with CF501 (at 122 days after the first vaccination), whereas the neutralizing antibody titer against Omicron pseudovirus in sera is 2602 at 122 days after the first vaccination of the rhesus monkeys with the RBD-Fc protein mixed with an aluminum adjuvant. In addition, the neutralizing antibody titers against the Omicron pseudovirus in sera of the rhesus monkeys vaccinated with the RBD-Fc protein mixed with CF501 are significantly higher than those in sera of the rhesus monkeys vaccinated with the RBD-Fc protein mixed with the aluminum adjuvant from 28 days to 191 days after the first vaccination.

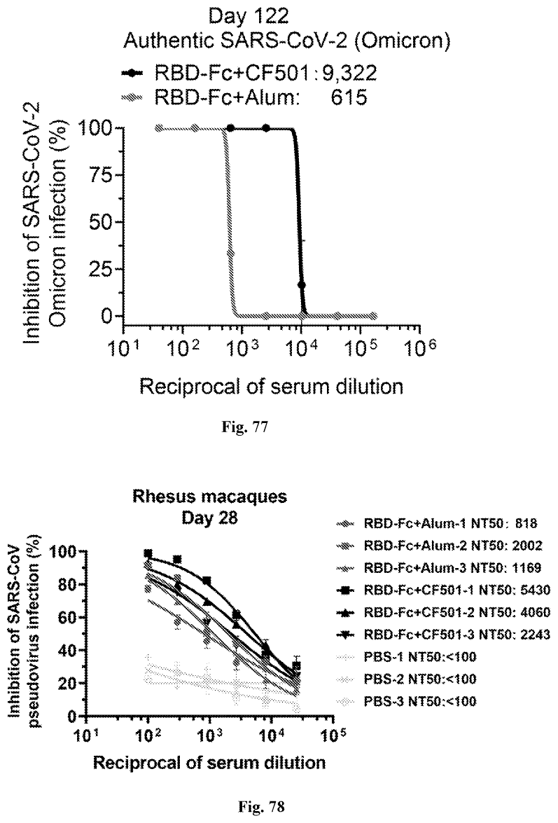

The neutralizing activities against the live Omicron virus in the sera at 122 days after the first vaccination of the rhesus monkeys were also detected. The results are shown in FIG. 77. The neutralizing antibody titer against the live Omicron virus in the sera is 9322 at 122 days after vaccination of the rhesus monkeys with the RBD-Fc protein mixed with CF501. The neutralizing antibody titer against the live Omicron virus in the sera is only 615 at 122 days after vaccination of the rhesus monkeys with the RBD-Fc protein mixed with the aluminum adjuvant. These results show that the vaccination with the RBD-Fc protein mixed with CF501 could strongly produce neutralizing antibodies against Omicron virus.

### Example 14: Evaluation of the Broad-Spectrum Neutralization Activities Against SARS-Related Viruses of the Sera from the Vaccinated Rhesus Monkeys

1. Packaging of SARS-Related Pseudoviruses (SARS-CoV, WIV1 and Rs3367).
(1) HEK-293T cells were co-transfected with the PCDNA-3.1-SARS-CoV-S, PCDN-3.1-WIV1-S or PCDNA-3.1-Rs3367-S plasmid and the HIV backbone plasmid PNL-4-3-Luc plasmid (see above).
(2) The cell supernatant containing the corresponding pseudovirus was collected after incubation for 48 hrs.

2. Evaluation of Inhibition of SARS-CoV, WIV1, and Rs3367 Pseudoviruses by Sera
(1) The rhesus monkey sera were diluted in 3 folds serially, and then the corresponding pseudovirus (SARS-CoV, WIV1 or Rs3367) was added. The mixture was incubated at 37° C. for 30 mins.
(2) The mixture was added to the Huh-7 cells which were already plated into a plate.
(3) After incubation for 12 hrs, the culture medium was exchanged with a fresh DMEM
(4) After incubation for 48 hrs, the cell lysis solution in the luciferase detection kit of Promega was used to lyse the cell, and the luciferase activity in the lysate was detected.

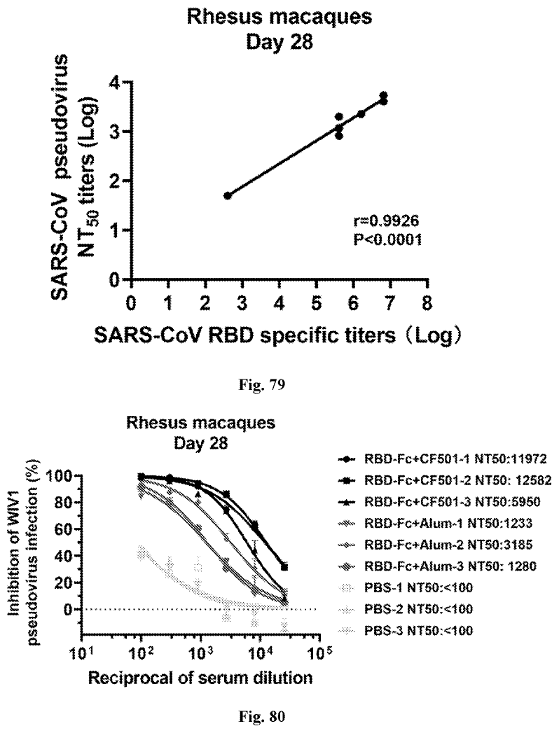

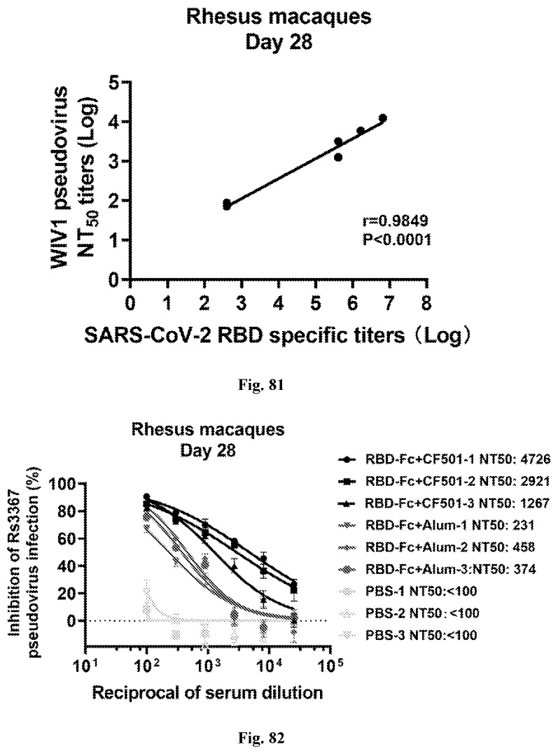

The results are shown in FIG. 78, which shows the neutralization activities of the sera from the vaccinated rhesus monkeys against the SARS-CoV pseudovirus. It can be seen that the sera from the three rhesus monkeys vaccinated with the RBD-Fc protein mixed with CF501 show the NT50 against the SARS-CoV pseudovirus of 5430, 4060, and 2243, respectively, while the sera from the three rhesus monkey vaccinated with the RBD-Fc protein mixed with the aluminum adjuvant show the NT50 against the SARS-CoV pseudovirus of 818, 2002 and 1189, respectively. The SARS-CoV-2 RBD specific antibodies in the sera from these 9 rhesus monkeys show a strong correlation with the SARS-CoV neutralizing antibody titers (FIG. 79). FIG. 80 shows the neutralization activities of the sera from the vaccinated rhesus monkeys against the WIV1 pseudovirus. It can be seen that the sera from the three rhesus monkeys vaccinated with the RBD-Fc protein mixed with CF501 show the NT50 against the SARS-CoV WIV1 pseudovirus of 11972, 12582 and 5950, respectively, while the sera from the three rhesus monkeys vaccinated with the RBD-Fc protein mixed with the aluminum adjuvant show the NT50 against the SARS-CoV WIV1 pseudovirus of 1233, 3185 and 1280, respectively. The SARS-CoV-2 RBD specific antibodies in the sera from these 9 rhesus monkeys show a strong correlation with the neutralizing antibody titers against the WIV1 pseudovirus (FIG. 81).

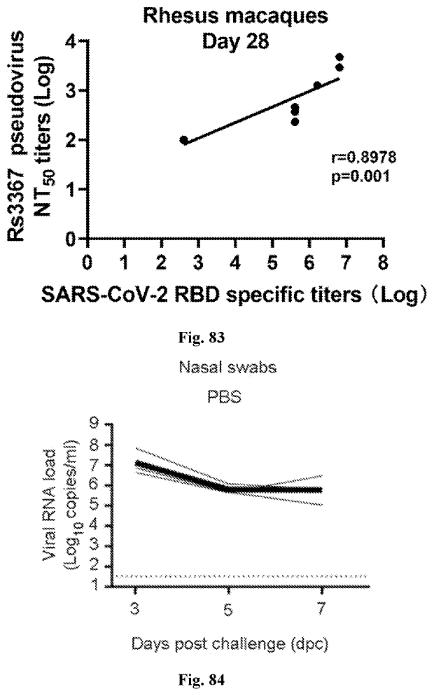

FIG. 82 shows the neutralization activity against the Rs3367 pseudovirus of the sera from the vaccinated rhesus monkeys. It can be seen that the sera from the three rhesus monkeys vaccinated with the RBD-Fc protein mixed with CF501 show the NT50 against the SARS-CoV pseudovirus of 4729, 2921, and 1267, respectively, while the sera from the three rhesus monkeys vaccinated with the RBD-Fc protein mixed with the aluminum adjuvant show the NT50 against the SARS-CoV pseudovirus of 231, 458, and 374, respectively. The SARS-CoV-2 RBD specific antibodies in the sera from these 9 rhesus monkeys show a strong correlation with the neutralizing antibody titers against Rs3367 (FIG. 83). As compared with the aluminum adjuvant, a more potent broadly-neutralizing antibody immune response can be produced in vaccinated monkeys when CF501 is used as an adjuvant for a COVID-19 vaccine.

### Example 15: Challenge Test with SARS-CoV-2 to Determine the Protective Effect after Vaccination of Rhesus Monkeys with the RBD Fc Protein Mixed with the Sting Agonist CF501

(1) At 223 days after the first vaccination of rhesus monkeys, the rhesus monkeys were challenged with SARS-CoV-2. Briefly, the rhesus monkeys were infected with 1 ml of SARS-CoV-2/WH-09/human/2020/CHN at a concentration of 10⁶ TCID50/ml by nasal drip.
(2) Nasal swabs were collected at 3, 5 and 7 days after the infection of the rhesus monkeys.
(3) At 7 days after the infection, the rhesus monkeys were euthanized and lungs, nasal turbinates and nasal mucosae were collected from the rhesus monkeys.
(4) RNA was extracted from the tissues with Trizol (Takara), and then the viral loads in the tissues of the rhesus monkeys were detected with a kit for RT-qPCR detection (Takara).

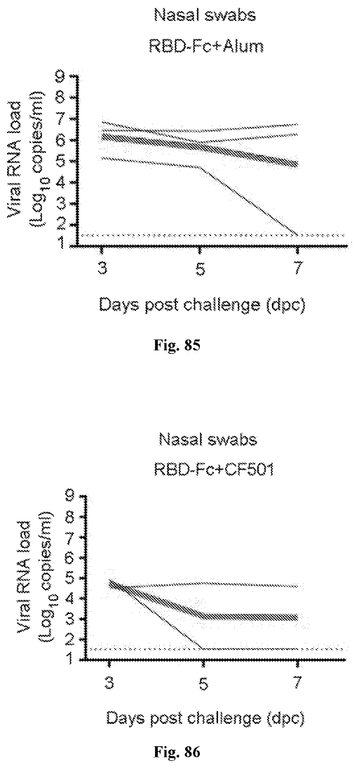

The results of viral loads in nasal swabs are shown in FIGS. 84, 85, 86 and Table 26. At 3, 5 and 7 days after the infection of the rhesus monkeys, the viral loads in nasal swabs of the rhesus monkeys vaccinated with PBS are higher. In one of the rhesus monkeys vaccinated with the RBD-Fc protein mixed with CF501, only a lower amount of SARS-CoV-2 RNA is detected in the nasal swabs at 3 days after the infection, and SARS-CoV-2 RNA is not detected at 5 and 7 days after the infection. SARS-CoV-2 RNA is detected in the nasal swabs of the other rhesus monkey vaccinated with the RBD-Fc protein mixed with CF501 at 3, 5 and 7 days after the infection, but the level of SARS-CoV-2 RNA in the rhesus monkey is significantly lower than that in the rhesus monkeys vaccinated with PBS. The copy number of SARS-CoV-2 RNA in the nasal swabs of the rhesus monkeys vaccinated with the RBD-Fc protein mixed with aluminum adjuvant is similar to that in the rhesus monkeys vaccinated with PBS at 3, 5 and 7 days after the infection.

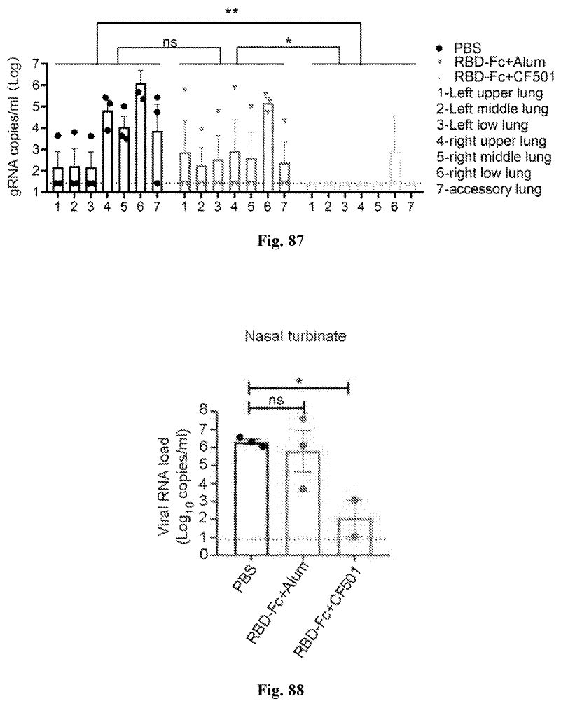

The results of viral loads in the lungs of the rhesus monkeys are shown in FIG. 87. A higher copy number of SARS-CoV-2 RNA can be detected in the left upper part, left middle part, left low part, right upper part, right middle part, right low part and lung accessory lobe in the lungs of the rhesus monkeys vaccinated with PBS or the RBD-Fc protein mixed with aluminum adjuvant. In one of the rhesus monkeys vaccinated with the RBD-Fc protein mixed with CF501, SARS-CoV-2 RNA is not detected in all lung lobes, and in the other rhesus monkey vaccinated with the RBD-Fc protein mixed with CF501, a lower copy number of SARS-CoV-2 RNA is detected in the right lower part of the lung. It shows that the vaccination with the RBD-Fc protein mixed with CF501 can reduce the infection of SARS-CoV-2 in the lungs as compared with the vaccination with PBS or the RBD-Fc protein mixed with the aluminum adjuvant.

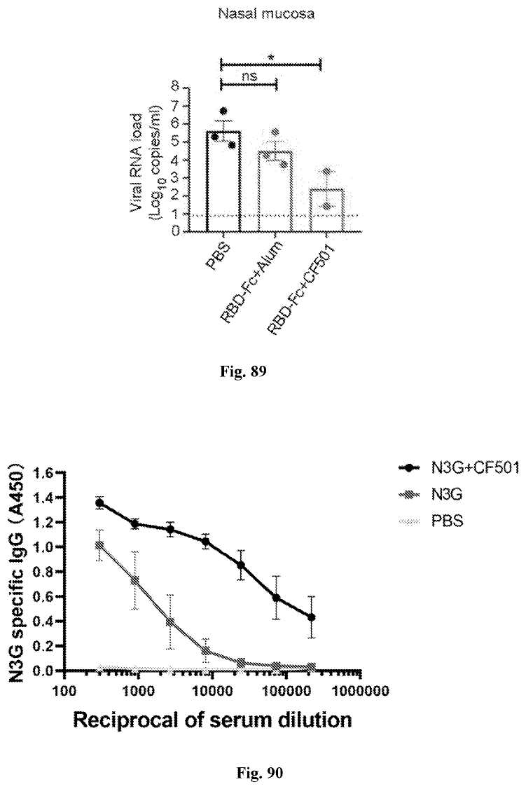

The viral loads in nasal mucosae and turbinates of the rhesus monkeys are shown in FIGS. 88 and 89 and Tables 27 and 28. The viral loads in the nasal turbinates and nasal mucosae of the rhesus monkeys vaccinated with the RBD-Fc protein mixed with CF501 are significantly lower than those in the rhesus monkeys vaccinated with PBS.

TABLE 26
Viral loads in nasal swabs of the rhesus monkeys at
3, 5 and 7 days after challenge with SARS-CoV-2

| Groups (mean | | | |
|---|---|---|---|
| virus copies | | | |
| for each group) | Day 3 | Day 5 | Day 7 |
| PBS | 13330240 | 633101 | 596613 |
| RBD-Fc mixed with | 1435555 | 465883 | 69850 |
| the aluminum | | | |
| adjuvant | | | |
| RBD-Fc mixed with | 55013 | 1401 | 1179 |
| CF501 | | | |

TABLE 27
Viral loads in nasal turbinates of the rhesus monkeys
at 7 days after challenge with SARS-CoV-2

| | Copy numbers of SARS-CoV-2 RNA | | |
|---|---|---|---|
| Groups | (copies for each animal) | | |
| PBS | 2060648 | 1151094 | 3702448 |
| RBD-Fc mixed with | 4815 | 1313759 | 40836921 |
| the aluminum | | | |
| adjuvant | | | |
| RBD-Fc mixed with | 11 | 1234 | — |
| CF501 | | | |

TABLE 28
Viral loads in nasal mucosae of the rhesus monkeys
at 7 days after challenge with SARS-CoV-2

| | Copy numbers of SARS-CoV-2 RNA | | |
|---|---|---|---|
| Groups | (copies for each animal) | | |
| PBS | 5464645 | 198507 | 67487 |
| RBD-Fc mixed with | 18650 | 347824 | 5457 |
| the aluminum | | | |
| adjuvant | | | |
| RBD-Fc mixed with | 26 | 2315 | — |
| CF501 | | | |

### Example 16: CF501 can Enhance the Immune Response to the HIV NHR Trimer

The Balb/c mice were divided into two groups. The first group was vaccinated with the HIV NHR trimer (N3G) (from Wang Chao who works in the Academy of Military Medical Sciences, China), and the second group was vaccinated with the HIV NHR trimer (N3G) and CF501. The mice were boosted once at 14 days after the first vaccination. The sera were collected from the mice at 7 days after the second vaccination. The specific antibodies to the HIV NHR trimer in the mice were tested by the ELISA method.

The results are shown in FIG. 90. CF501 can still be used as an adjuvant for the HIV NHR trimer and can significantly enhance the antibody immune response in vaccinated mice. CF501 can not only be used in a COVID-19 vaccine, but also effectively enhance the immune response to the polypeptide antigen of HIV.

### Example 17: CF501 can Enhance the Immune Response to the Inactivated Influenza Virus Quadrivalent Vaccine

1. Balb/c mice were divided into three groups of 6 mice each. The first group was only vaccinated with the inactivated influenza virus quadrivalent vaccine (Hualan Bio Co., Ltd.) (comprising influenza subtypes H1N1, H3N2, B/Yamagata, and B/Victoria). The second group was vaccinated with the aluminum adjuvant and the inactivated influenza virus quadrivalent vaccine. The third group was vaccinated with CF501 and the inactivated influenza virus quadrivalent vaccine. The sera were collected at 14 days after the vaccination. The second booster vaccination was performed at Day 21 after the first vaccination, and the sera were collected at 28 days after the first vaccination.
2. Evaluation of influenza virus HA specific antibody titers in sera from the vaccinated mice
1) HA proteins of the four influenza virus subtypes corresponding to the virus subtypes in the vaccine (subtypes H1N1, H3N2, B/Yamagata, and B/Victoria, respectively, purchased from Sino biological Inc.) were coated onto an ELISA plate respectively.
2) PBST was used to dilute the mouse sera in 3 or 5 folds serially, and then the diluted sera were added to the coated ELISA plate.
3) After incubation at 37° C. for 1 hr, the plate was washed with PBST for 5 times, and the rabbit anti-mouse HRP secondary antibody (Dako, 1:2000 dilution) was added.
4) After incubation at 37° C. for 1 hr, the plate was washed with PBST for 5 times, the TMB (Sigma) substrate was added for color development, and H2SO₄ was added to stop the reaction. The OD450 was read on a microplate reader.

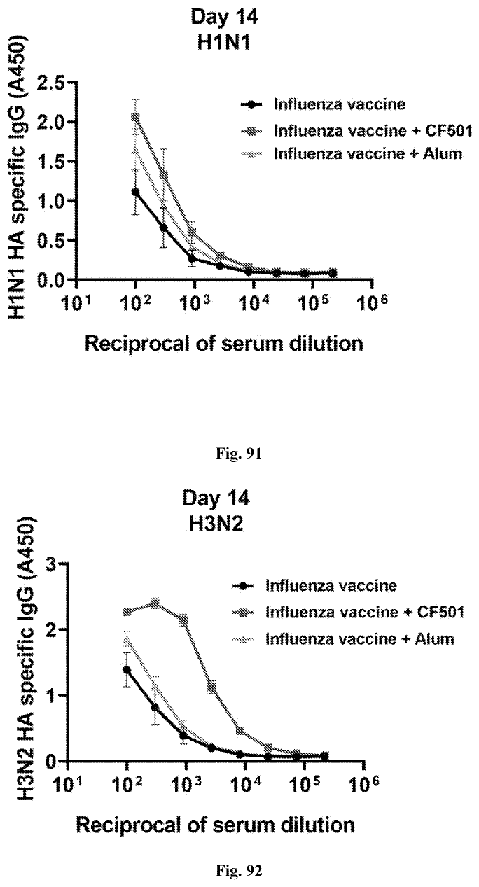

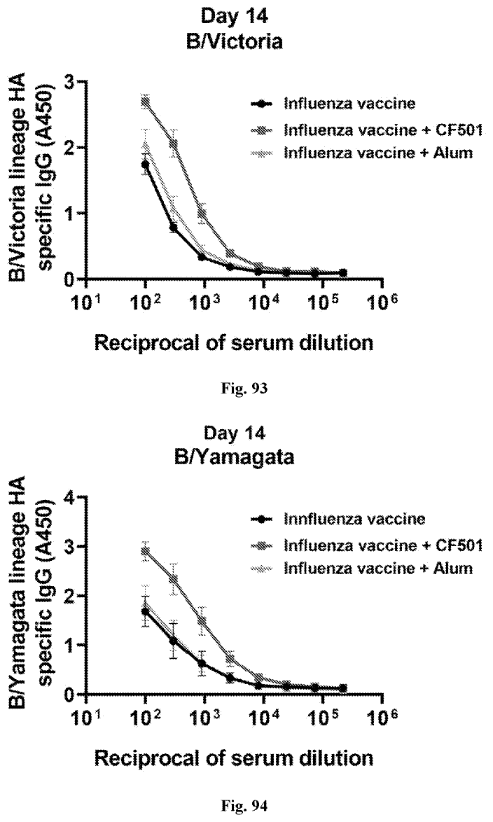

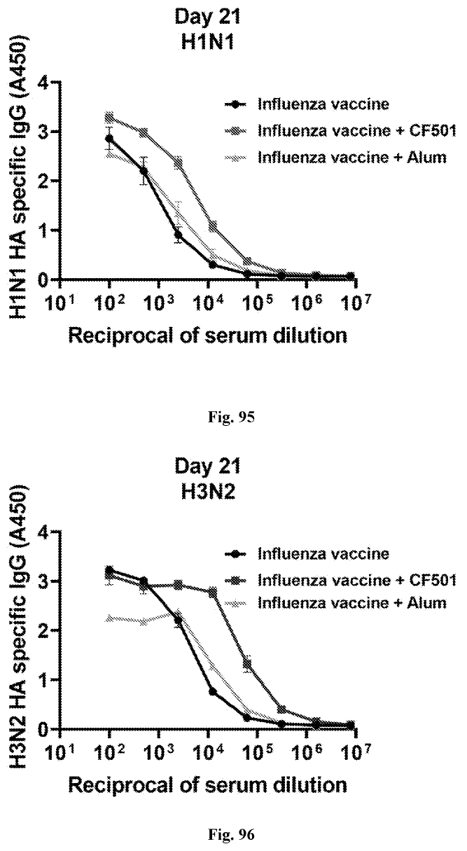

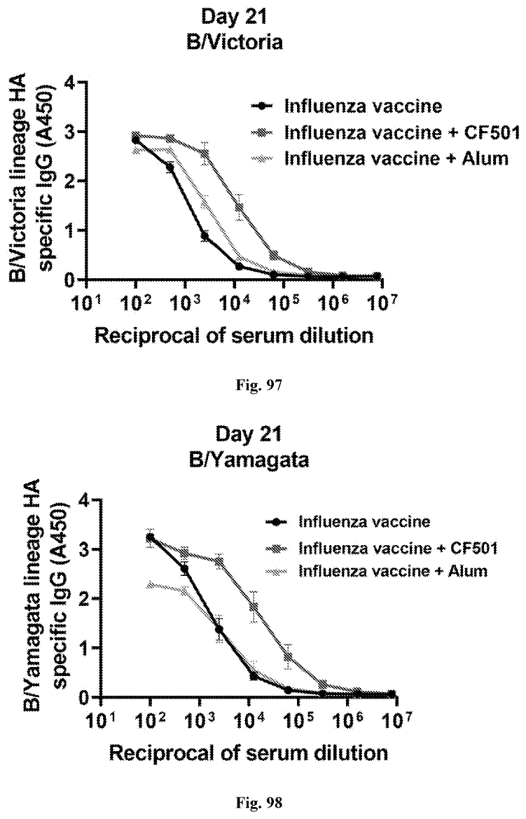

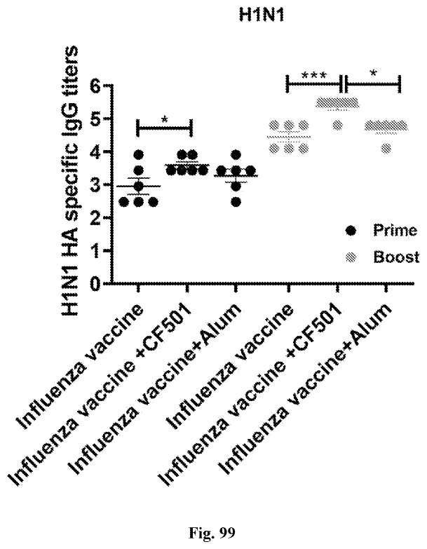

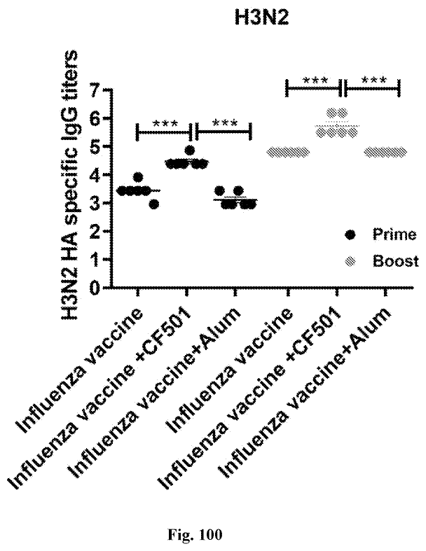

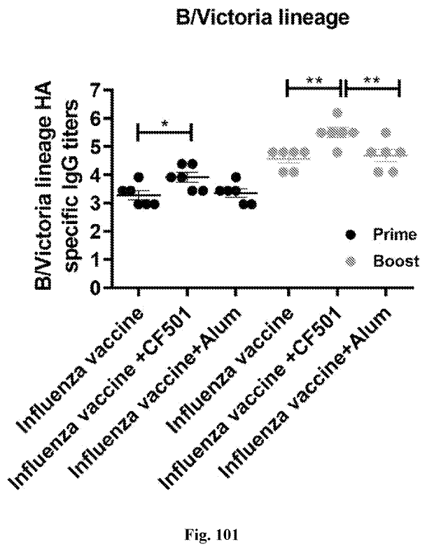

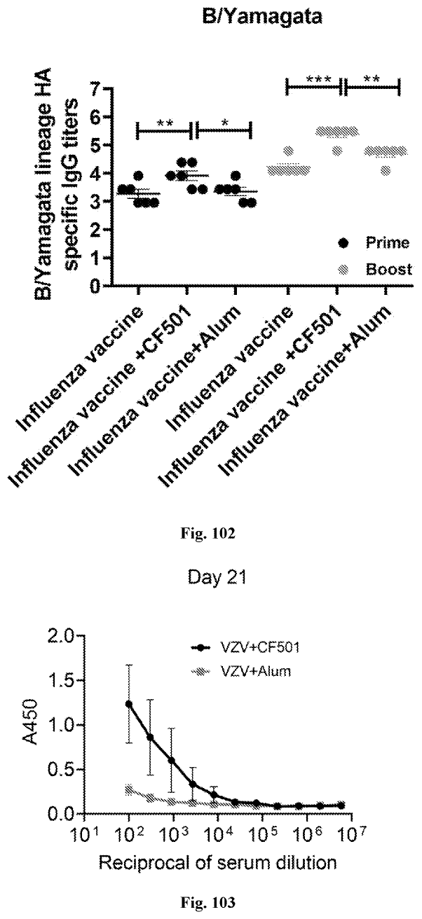

The results are shown in FIGS. 91-102 and Tables 29-36. Among the three groups of mice, whether at Day 14 or Day 28, the antibody titers against the HA proteins of the four influenza virus subtypes in the sera from the mice vaccinated with the quadrivalent inactivated influenza virus vaccine and CF501 are highest, which are significantly higher than those in the sera from the mice vaccinated with the quadrivalent inactivated influenza virus vaccine without an adjuvant and the mice vaccinated with the quadrivalent inactivated influenza virus vaccine and the aluminum adjuvant.

TABLE 29
Antibody titers against the HA protein of the H1N1 subtype in sera at Day 14 after
the vaccination of the mice with the quadrivalent inactivated influenza vaccine

| | quadrivalent | quadrivalent | quadrivalent |
|---|---|---|---|
| | inactivated | inactivated | inactivated influenza |
| Group of | influenza | influenza | vaccine + aluminium |
| mice | vaccine | vaccine + CF501 | adjuvant |
| HA-specific | 2100 ± 1258 | 4500 ± 1138 | 2900 ± 1124 |
| antibody titers | | | |
| Significance | P = 0.0197 | | P = 0.3046 |
| analysis (Compared | | | |
| with CF501 | | | |
| group) | | | |

TABLE 30
Antibody titers against the HA protein of the H3N2 subtype in sera at Day 14 after
the vaccination of mice with the quadrivalent inactivated influenza vaccine

| | Quadrivalent | Quadrivalent | Quadrivalent |
|---|---|---|---|
| | inactivated | inactivated | inactivated influenza |
| the group | influenza | influenza | vaccine + aluminium |
| of mice | vaccine | vaccine + CF501 | adjuvant |
| HA specific | 3300 ± 1003 | 32400 ± 8100 | 1500 ± 379 |
| antibody titer | | | |
| Significance | P < 0.0001 | | P < 0.0001 |
| analysis(Compared | | | |
| with CF501 | | | |
| group) | | | |

TABLE 31
Antibody titers against the HA protein of the B/Yamagata
subtype in sera at Day 14 after the vaccination of mice
with the quadrivalent inactivated influenza vaccine

| | Quadrivalent | Quadrivalent | Quadrivalent |
|---|---|---|---|
| | inactivated | inactivated | inactivated influenza |
| Group of | influenza | influenza | vaccine + aluminium |
| mice | vaccine | vaccine + CF501 | adjuvant |
| HA specific | 2700 ± 1138 | 11700 ± 4104 | 3000 ± 081 |
| antibody titer | | | |
| Significance | P = 0.0060 | | P = 0.0163 |
| analysis | | | |
| (Compared | | | |
| with CF501 | | | |
| group) | | | |

TABLE 32
Antibody titers against the HA protein of the B/Victoria
subtype in sera at Day 14 after the vaccination of mice
with the quadrivalent inactivated influenza vaccine

| | Quadrivalent | Quadrivalent | Quadrivalent |
|---|---|---|---|
| | inactivated | inactivated | inactivated influenza |
| Group of | influenza | influenza | vaccine + aluminium |
| mice | vaccine | vaccine + CF501 | adjuvant |
| HA specific | 2700 ± 1138 | 11700 ± 4104 | 3000 ± 1081 |
| antibody titer | | | |
| Significance | P = 0.0060 | | P = 0.0163 |
| analysis | | | |
| (Compared | | | |
| with CF501 | | | |
| group) | | | |

TABLE 33
Antibody titers against the HA protein of the H1N1 subtype
in sera at Day 28 after the vaccination of mice with
the quadrivalent inactivated influenza vaccine

| | Quadrivalent | Quadrivalent | Quadrivalent |
|---|---|---|---|
| | inactivated | inactivated | inactivated influenza |
| Group of | influenza | influenza | vaccine + aluminium |
| mice | vaccine | vaccine + CF501 | adjuvant |
| HA specific | 37500 ± 11180 | 270833 ± 41666 | 54166 ± 8333 |
| antibody titer | | | |
| Significance | P = 0.0008 | | P = 0.0102 |
| analysis | | | |
| (Compared | | | |
| with CF501 | | | |
| group) | | | |

TABLE 34
Antibody titers against the HA protein of the H3N2 subtype
in sera at Day 28 after the vaccination of mice with
the quadrivalent inactivated influenza vaccine

| | Quadrivalent | Quadrivalent | Quadrivalent |
|---|---|---|---|
| | inactivated | inactivated | inactivated influenza |
| Group of | influenza | influenza | vaccine + aluminium |
| mice | vaccine | vaccine + CF501 | adjuvant |
| HA specific | 62500 ± 0 | 729166 ± 263523 | 62500 ± 0 |
| antibody titer | | | |
| Significance | P < 0.0001 | | P < 0.0001 |
| analysis | | | |
| (Compared | | | |
| with CF501 | | | |
| group) | | | |

TABLE 35
Antibody titers against the HA protein of the B/Yamagata
subtype in sera at Day 28 after the vaccination of mice
with the quadrivalent inactivated influenza vaccine

| | Quadrivalent | Quadrivalent | Quadrivalent |
|---|---|---|---|
| | inactivated | inactivated | inactivated influenza |
| Group of | influenza | influenza | vaccine + aluminium |
| mice | vaccine | vaccine + CF501 | adjuvant |
| HA specific | 20833 ± 8333 | 270833 ± 41666 | 54166 ± 8333 |
| antibody titer | | | |
| Significance | P < 0.0001 | | P = 0.0026 |
| analysis | | | |
| (Compared | | | |
| with CF501 | | | |
| group) | | | |

TABLE 36
Antibody titers against the HA protein of the B/Victoria
subtype in sera at Day 28 after the vaccination of mice
with the quadrivalent inactivated influenza vaccine

| | Quadrivalent | Quadrivalent | Quadrivalent |
|---|---|---|---|
| | inactivated | inactivated | inactivated influenza |
| Group of | influenza | influenza | vaccine + aluminium |
| mice | vaccine | vaccine + CF501 | adjuvant |
| HA specific | 45833 ± 10540 | 479166 ± 220479 | 87500 ± 46097 |
| antibody titer | | | |
| Significance | P = 0.0017 | | P = 0.0060 |
| analysis | | | |
| (Compared | | | |
| with CF501 | | | |
| group) | | | |

### Example 18: The Sting Agonist CF501 can Enhance the Immune Response to a Varicella Zoster Virus (VZV) Inactivated Vaccine

Materials: The VZV inactivated vaccine is commercially available from Sinovac (Dalian) Biotech Ltd.
1) The mice were divided into two groups with 6 mice in each group.
2) The first group of mice were vaccinated intramuscularly with 20 μg of CF501 and the VZV inactivated vaccine containing 10 ng of gE protein.
3) The second group of mice were vaccinated intramuscularly with 200 μg of the aluminum adjuvant and the VZV inactivated vaccine containing 10 ng of gE protein.
4) The mice were vaccinated at Days 0, 14 and 28, and sera were collected from the mice at Days 21 and 35.
5) An ELISA test was used to determine the levels of antibodies specifically binding to the VZV gE protein in the sera of mice at Days 21 and 35.

In particular, the ELISA test was performed as follows.
A. The wells of an ELISA plate were coated with 1 μg/ml of the gE protein with 50 μl per well, and the plate was incubated at 4° C. overnight. The plate was blocked with PBS containing 5% BSA at 37° C. for 2 hrs.
C. The sera of the mice were diluted in 100 folds initially, then diluted in 10 folds serially, and added to the ELISA plate. The plate was incubated at 37° C. for 45 mins.
D. After the wells of the plate were washed with PBST for 5 times, the HRP labeled rabbit anti-mouse IgG was added and the plate was incubated at 37° C. for 45 mins.
E. After the wells of the plate were washed with PBST for 5 times, the TMB substrate was added for color development for 15 mins. H₂SO₄ was added to stop the color development.
F. OD450 was measured by a microplate reader.

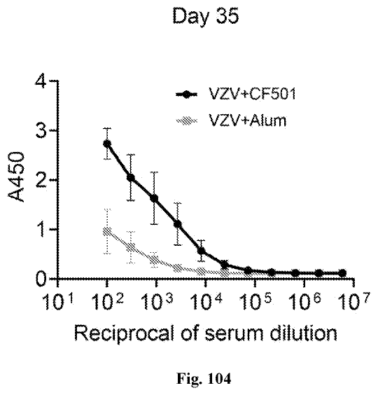

Results as shown in FIGS. 103 and 104. The VZV inactivated vaccine with CF501 as the adjuvant can produce a more potent antibody immune response in mice as compared to the VZV inactivated vaccine with the aluminum adjuvant as an adjuvant. These results fully show that CF501 can be used as a general adjuvant to stimulate more potent immune responses to different virus subunits and inactivated vaccines.

1. A compound having formula (I) or pharmaceutically acceptable salts thereof, 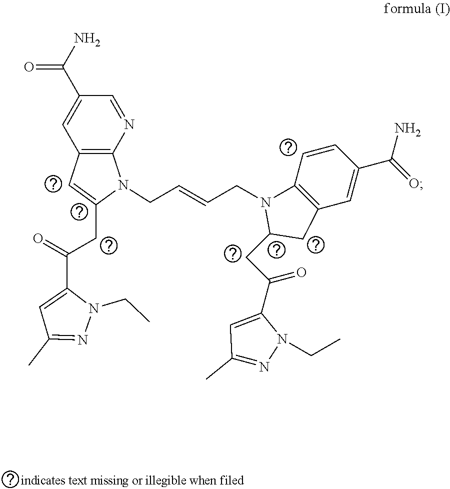
wherein R₁ is CR₁′, wherein R₁′ is H, —OMe or —O(CH₂)ₙNR₂′R₃′, n is an integer of 1 to 6, preferably 2 or 3, R₂′ and R₃′ taken together with the nitrogen atom through which they are connected to form a substituted or unsubstituted 5-6 membered heterocycloalkyl or substituted or unsubstituted 5-6 membered heteroaryl, wherein the substituted 5-6 membered heterocycloalkyl or substituted 5-6 membered heteroaryl is independently substituted with one or more halogen, OH, amine, CN, CF₃, or unsubstituted C₁-C₄ saturated alkyl; preferably, the heterocycloalkyl is one of: 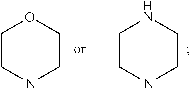
wherein R₂ and R₃ each independently are N or NR₄′, wherein R₄′ is H or C₁₋₄ saturated alkyl; and
wherein R₄ and R₅ each independently are N or NH.

2. The compound of claim 1 or pharmaceutically acceptable salts thereof, wherein the compound has the structure:

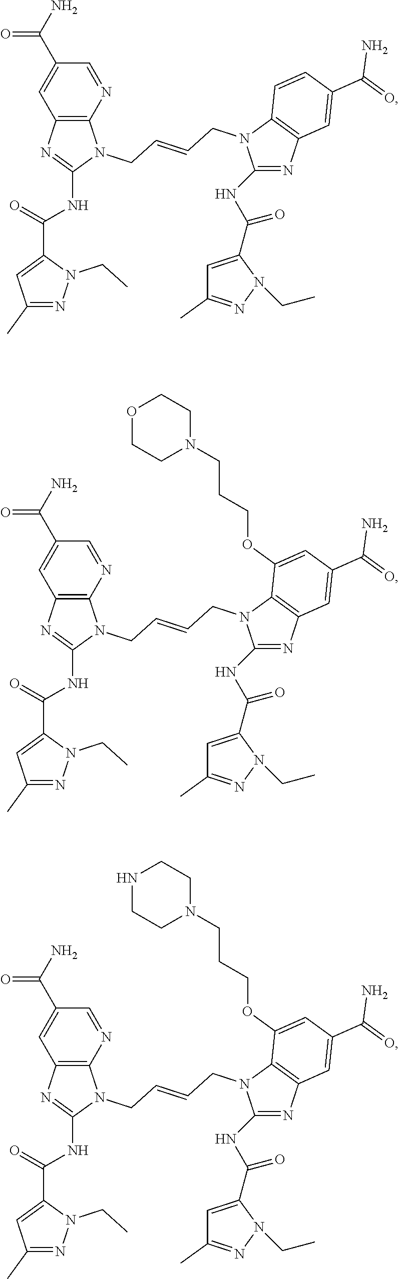 [SMILES: CCn1nc(C)cc1C(=O)Nc1nc2cc(C(N)=O)ccc2n1C/C=C/Cn1c(NC(=O)c2cc(C)nn2CC)nc2cc(C(N)=O)cnc21 ; CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN3CCOCC3)c21 ; CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN3CCNCC3)c21]

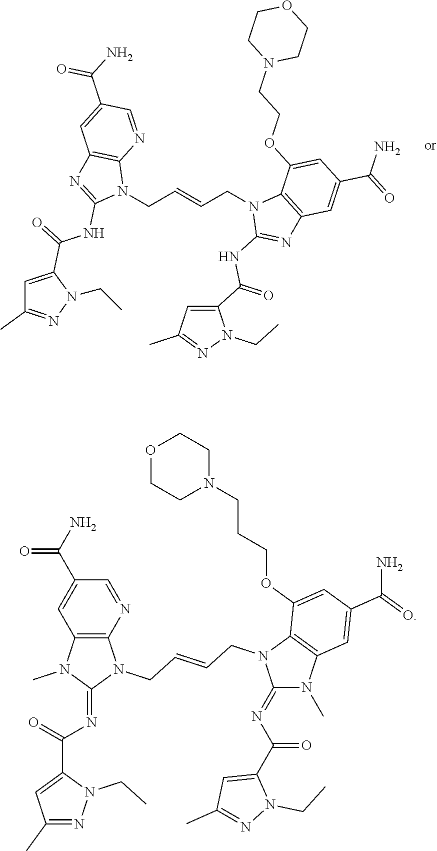 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCN3CCOCC3)c21 ; "or" ; CCn1nc(C)cc1C(=O)/N=c1\n(C)c2cc(C(N)=O)cnc2n1C/C=C/Cn1/c(=N/C(=O)c2cc(C)nn2CC)n(C)c2cc(C(N)=O)cc(OCCCN3CCOCC3)c21]

3. A pharmaceutical composition, comprising
the compound of claim 1 or pharmaceutically acceptable salts thereof, and
at least one of a pharmaceutically acceptable carrier, a pharmaceutically acceptable excipient, and a pharmaceutically acceptable diluent.

4. Use of the compound of claim 1 or pharmaceutically acceptable salts thereof or the pharmaceutical composition of claim 3 for the manufacture of an adjuvant, preferably wherein the adjuvant is an adjuvant for a vaccine, such as inactivated vaccine, live-attenuated vaccine, subunit vaccine, nucleic acid vaccine such as mRNA or DNA vaccine.

5. The use of claim 4, wherein the vaccine comprises an antigen selected from a group consisting of a cancer antigen, a viral antigen, a bacterial antigen, a parasitic antigen, and a fungal antigen.

6. The use of claim 5, wherein the viral antigen is selected from a group consisting of an HIV antigen, an influenza antigen, and a coronavirus antigen, preferably, antigens from one or more of HCoV-229E, HCoV-OC43, SARS-CoV, HCoV-NL63, HCoV-HKU1, MERS-CoV, Varicella-zoster virus (VZV) and SARS-CoV-2 such as SARS-CoV-2 Omicron mutant, preferably SARS-CoV-2 RBD-Fc protein or gE protein of Varicella zoster virus.

7. A vaccine, comprising
the compound of claim 1 or pharmaceutically acceptable salts thereof; and
an antigen, preferably wherein the vaccine is an intramuscular, an intradermal vaccine or an inhaled vaccine.

8. The vaccine of claim 7, wherein the vaccine comprises an antigen selected from a group consisting of a cancer antigen, a viral antigen, a bacterial antigen, a parasitic antigen, and a fungi antigen, preferably wherein the vaccine is an inactivated vaccine, live-attenuated vaccine, subunit vaccine, nucleic acid vaccine such as mRNA or DNA vaccine.

9. The vaccine of claim 8, wherein the viral antigen is selected from a group consisting of an HIV antigen, an influenza antigen, and a coronavirus antigen, preferably an antigen from one or more of HCOV-229E, HCOV-OC43, SARS-COV, HCOV-NL63, HCOV-HKU1, MERS-COV, Varicella-zoster virus (VZV) and SARS-COV-2 such as SARS-CoV-2 Omicron mutant, preferably SARS-CoV-2 RBD-Fc protein or gE protein of Varicella zoster virus.

10. A method for producing of the vaccine of claim 7, comprising mixing the compound of claim 1 and an antigen.

11. The compound of claim 1 or pharmaceutically acceptable salts thereof for use as an adjuvant, preferably wherein the adjuvant is an adjuvant for a vaccine preferably wherein the vaccine is an inactivated vaccine, live-attenuated vaccine, subunit vaccine, nucleic acid vaccine such as mRNA or DNA vaccine.

12. The compound or pharmaceutically acceptable salts thereof for the use according to claim 11, wherein the vaccine comprises an antigen selected from a group consisting of a cancer antigen, a viral antigen, a bacterial antigen, a parasitic antigen, and a fungi antigen.

13. The compound or pharmaceutically acceptable salts thereof for the use according to claim 12, wherein the viral antigen is selected from a group consisting of an HIV antigen, an influenza antigen, and a coronavirus antigen, preferably an antigen from one or more of, HCOV-229E, HCOV-OC43, SARS-COV, HCOV-NL63, HCOV-HKU1, MERS-COV, Varicella-zoster virus (VZV) and SARS-COV-2 such as SARS-CoV-2 Omicron mutant, preferably SARS-CoV-2 RBD-Fc protein or gE protein of Varicella zoster virus.

14. A method for treating or preventing an infectious disease or a cancer, which comprises administering an effective amount of the vaccine of claim 7 to a subject in need thereof, preferably wherein the vaccine is an intramuscular, intradermal vaccine or inhaled vaccine preferably wherein the vaccine is an inactivated vaccine, live-attenuated vaccine, subunit vaccine, nucleic acid vaccine such as mRNA or DNA vaccine.

15. The method of claim 14, wherein
the infectious disease is selected from a group consisting of AIDS, severe acute respiratory syndrome (SARS), Middle East respiratory syndrome (MERS), COVID-19, Varicella zoster and influenza, and
the cancer is selected from a group consisting of HPV-related cancer, HBV-related cancer, ovarian cancer, prostate cancer, breast cancer, brain cancer, head and neck cancer, laryngeal cancer, lung cancer, liver cancer, pancreatic cancer, kidney cancer, bone cancer, melanoma, metastatic cancer, HTERT-related cancer, FAP antigen-related cancer, non-small cell lung cancer, blood cancer, esophageal squamous cell carcinoma, cervical cancer, bladder cancer, colorectal cancer, gastric cancer, anal cancer, synovial sarcoma, testicular cancer, recurrent respiratory system papillomatosis, skin cancer, glioblastoma, liver cancer, gastric cancer, acute myeloid leukemia, triple-negative breast cancer, and primary cutaneous T-cell lymphoma.

16. A kit comprising the compound of claim 1, an antigen, and instructions for treating or preventing an infectious disease or a cancer.
